(19) United States
(12) Patent Application Publication
(10) Pub. No.: US 2021/0317141 A1
(43) Pub. Date: Oct. 14, 2021

(54) OXOPYRIDO[1,2-A]PYRIMIDINE COMPOUNDS FOR THE TREATMENT AND PROPHYLAXIS OF BACTERIAL INFECTION

(71) Applicant: Hoffmann-La Roche Inc., Little Falls, NJ (US)

(72) Inventors: Fabian DEY, Basel (CH); Xiao DING, Shanghai (CN); Yimin HU, Shanghai (CN); Yongqiang LIU, Shanghai (CN); Hong SHEN, Shanghai (CN); Honguang SHI, Shanghai (CN); Xuefei TAN, Shanghai (CN); Chengang ZHOU, Shanghai (CN); Mingwei ZHOU, Shanghai (CN)

(73) Assignee: Hoffmann-La Roche Inc., Little Falls, NJ (US)

(21) Appl. No.: 17/351,103

(22) Filed: Jun. 17, 2021

Related U.S. Application Data
(63) Continuation of application No. PCT/EP2019/086151, filed on Dec. 19, 2019.

(30) Foreign Application Priority Data
Dec. 20, 2018 (CN) PCT/CN2018/122365

(51) Int. Cl. C07D 519/00; A61P 31/04

(52) U.S. Cl. CPC C07D 519/00; A61P 31/04

(57) ABSTRACT

The present invention relates to novel compounds of formula (I),

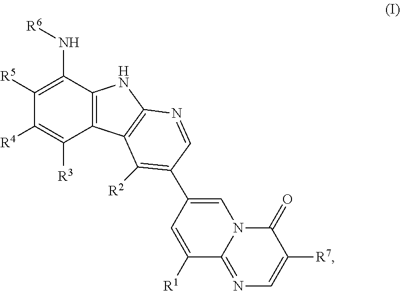

wherein R¹ to R⁷ are as described herein, and their pharmaceutically acceptable salt, enantiomer or diastereomer thereof, and compositions including the compounds and methods of using the compounds.

### CROSS REFERENCE TO RELATED APPLICATIONS

This application is a continuation of International Application No. PCT/EP2019/086151 having an International filing date of Dec. 19, 2019, which claims benefit of priority to International Application No. PCT/CN2018/122365 having an International filing date of Dec. 20, 2018, all of which are incorporated by reference in their entirety.

### BACKGROUND OF THE INVENTION

The present invention relates to organic compounds useful for therapy and/or prophylaxis in a mammal, and in particular to inhibitors of DNA gyrase and/or topoisomerase IV useful for treatment and/or prophylaxis of bacterial infection.

### FIELD OF THE INVENTION

Bacterial infections pose a continuing medical problem because anti-bacterial drugs eventually engender resistance in the bacteria on which they are used. Bacterial resistance against virtually all current antibiotic drugs are increasing. Many forms of antibiotic resistance can even cross international boundaries and spread with remarkable speed. Thus novel classes of antibacterial compounds are urgently needed.

One target for development of anti-bacterial drugs has been DNA gyrase and topoisomerase IV (bacterial type IIA topoisomerases), which are essential to cell life, that solve DNA topological problems resulting from the replication, transcription, and recombination of DNA. DNA Gyrase controls DNA supercoiling and relieves topological stress that occurs when the DNA strands are untwisted such as during replication. Topoisomerase IV primarily resolves linked chromosome dimers at the conclusion of DNA replication. Both enzymes can introduce double stranded DNA breaks; pass a second DNA strand through the break and rejoining the broken strands. The activity of both enzymes is driven by the binding and hydrolysis of ATP. Bacterial DNA gyrase consists of two A (GyrA) and two B (GyrB) subunits. Binding and cleavage of the DNA is associated with GyrA, whereas ATP is bound and hydrolyzed by GyrB. Bacterial Topoisomerase IV is also a hetero-tetramer that consists of two C (ParC) and two E (ParE) subunits. The latter subunits bind ATP like GyrB in order to supply energy necessary for catalytic turnover of the enzymes.

Inhibition of DNA gyrase and topoisomerase IV has potential for the development of broad-spectrum antibiotics. The enzymes are highly conserved across a broad range of gram-positive and gram-negative pathogens. There are two classes of antibiotics that demonstrated such mechanism of action. The first, well-represented by the quinolones, inhibits GyrA and ParC subunits by stabilizing the cleaved DNA-enzyme complex, thus inhibiting overall gyrase function, leading to cell death. Novobiocin, the only marketed drug in the second class, exerts its effect by blocking the ATPase activity of the enzymes. Novobiocin was identified in 1950s. But its use declined rapidly and it was eventually withdrawn from the market, mainly due to its low permeability in many bacteria strains, rise of spontaneous resistance development, and the development of more effective drugs, such as penicillinase-stable penicillins and the first cephalosporins in 1960s and 1970s.

Recently, strong inhibition of DNA gyrase and/or topoisomerase IV has been recognized to be important for low resistance development in bacterial strains treated by inhibitors of the enzymes. Inhibitors of bacterial DNA gyrase and/or topoisomerase IV with different mechanism of action compare to the widely used quinolones will exhibit minimal cross resistance, and will be potentially useful in combating quinolone resistance that has increased significantly in the past few years.

### SUMMARY OF THE INVENTION

The present invention relates to novel compounds of formula (I),

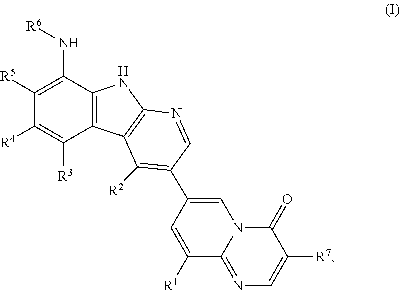

wherein
R¹ is H; (C₁₋₆alkyl)₂aminoC₁₋₆alkyl; C₁₋₆alkoxy; C₁₋₆ alkoxyC₁₋₆alkyl(C₁₋₆alkyl)aminoC₁₋₆alkyl; C₁₋₆alkyl; C₁₋₆alkyl-2,3,3a,4,6,6a-hexahydropyrrolo[2,3-c]pyrrolylC₁₋₆alkyl; morpholinyl; morpholinylC₁₋₆alkyl or pyrrolidinylC₁₋₆alkyl;
R² is ((C₁₋₆alkyl)₂amino)C₁₋₆alkylhalopyrrolidinyl; ((C₁₋₆alkyl)₂amino)C₁₋₆alkylpyrrolidinyl; C₁₋₆alkyl)₂amino; (C₁₋₆alkyl)₂aminopyrrolidinyl; C₁₋₆ alkoxyC₁₋₆ alkyl(C₁₋₆ alkyl)amino; C₁₋₆alkoxyC₁₋₆alkyl(C₁₋₆alkyl)aminoC₁₋₆alkylpyrrolidinyl; C₁₋₆alkyl-2,3,3a,4,6,6a-hexahydropyrrolo[2,3-c]pyrrolyl; C₃₋₇ cycloalkyl; C₃₋₇cycloalkyl(C₁₋₆alkyl)aminoC₁₋₆alkylpyrrolidinyl; cyano; haloC₁₋₆alkyl(C₁₋₆alkyl)amino; C₁₋₆alkyl)aminoC₁₋₆alkylpyrrolidinyl; haloC₁₋₆alkylpyrazolyl; halopyrrolidinyl; hexahydropyrazino[2,1-c][1,4]oxazin-8(1H)-yl; morpholinyl; phenylpyrrolidinyl or pyrrolidinyl;
R³ is H or halogen;
R⁴ is H or halogen;
R⁵ is H or halogen;
R⁶ is C₁₋₆alkyl;
R⁷ is carboxy;

or pharmaceutically acceptable salt thereof.

Objects of the present invention are novel compounds of formula (I), their manufacture, medicaments based on a compound in accordance with the invention and their production as well as the use of compounds of formula (I) for the treatment or prophylaxis of bacterial infection. The use of compounds of formula (I) as DNA gyrase and/or topoisomerase IV inhibitors is also one of the objections of present invention. The compounds of formula (I) showed superior anti-bacterial activity, good solubility, good CC₅₀ profiles, improved microsomal stability and/or improved PK profile.

### DETAILED DESCRIPTION OF THE INVENTION

Unless otherwise defined, all technical and scientific terms used herein have the same meaning as commonly understood by one of ordinary skill in the art to which this invention belongs. Furthermore, the following definitions are set forth to illustrate and define the meaning and scope of the various terms used to describe the invention.

### Definitions

The term “C₁₋₆alkyl” denotes a saturated, linear or branched chain alkyl group containing 1 to 6, particularly 1 to 4 carbon atoms, for example methyl, ethyl, propyl, isopropyl, n-butyl, isobutyl, tert-butyl and the like. Particular “C₁₋₆alkyl” groups are methyl, ethyl and propyl.

The term “C₁₋₆ alkoxy” denotes a group of the formula C₁₋₆alkyl-O—. Examples of C₁₋₆alkoxy group include, but not limited to, methoxy, ethoxy, n-propoxy, isopropoxy, n-butoxy, isobutoxy and tert-butoxy. Particular “C₁₋₆alkoxy” groups are methoxy, ethoxy and isopropoxy.

The term “halogen” and “halo” are used interchangeably herein and denote fluoro, chloro, bromo, or iodo.

The term “haloC₁₋₆alkyl” denotes an alkyl group wherein at least one of the hydrogen atoms of the alkyl group has been replaced by same or different halogen atoms, particularly fluoro atom. Examples of haloC₁₋₆ alkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl, difluoromethyl, trifluoromethyl and trifluoroethyl.

The term “halopyrrolidinyl” denotes a pyrrolidinyl substituted once, twice or three times by halogen. Examples of halopyrrolidinyl include, but not limited to, difluoropyrrolidinyl.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

Inhibitors of DNA Gyrase and/or Topoisomerase IV

The present invention relates to a compound of formula (I),

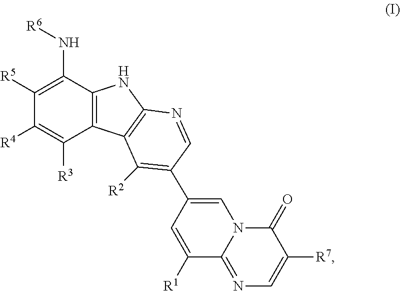

wherein
R¹ is H; (C₁₋₆alkyl)₂aminoC₁₋₆alkyl; C₁₋₆ alkoxy; C₁₋₆alkoxyC₁₋₆alkyl(C₁₋₆alkyl)aminoC₁₋₆alkyl; C₁₋₆alkyl; C₁₋₆alkyl-2,3,3a,4,6,6a-hexahydropyrrolo[2,3-c]pyrrolylC₁₋₆alkyl; morpholinyl; morpholinylC₁₋₆alkyl or pyrrolidinylC₁₋₆alkyl;
R² is ((C₁₋₆alkyl)₂amino)C₁₋₆alkylhalopyrrolidinyl; ((C₁₋₆alkyl)₂amino)C₁₋₆alkylpyrrolidinyl; C₁₋₆alkyl)₂amino; (C₁₋₆alkyl)₂aminopyrrolidinyl; C₁₋₆ alkoxyC₁₋₆ alkyl(C₁₋₆alkyl)amino; C₁₋₆alkoxyC₁₋₆alkyl(C₁₋₆alkyl)aminoC₁₋₆alkylpyrrolidinyl; C₁₋₆alkyl-2,3,3a,4,6,6a-hexahydropyrrolo[2,3-c]pyrrolyl; C₃₋₇ cycloalkyl; C₃₋₇cycloalkyl(C₁₋₆alkyl)aminoC₁₋₆alkylpyrrolidinyl; cyano; haloC₁₋₆alkyl(C₁₋₆alkyl)amino; haloC₁₋₆alkyl(C₁₋₆alkyl)aminoC₁₋₆ alkylpyrrolidinyl; haloC₁₋₆alkylpyrazolyl; halopyrrolidinyl; hexahydropyrazino[2,1-c][1,4]oxazin-8(1H)-yl; morpholinyl; phenylpyrrolidinyl or pyrrolidinyl;
R³ is H or halogen;
R⁴ is H or halogen;
R⁵ is H or halogen;
R⁶ is C₁₋₆alkyl;
R⁷ is carboxy;
   or pharmaceutically acceptable salt thereof.

A further embodiment of present invention is (ii) a compound of formula (I), wherein
R¹ is H; dimethylaminomethyl; methoxy; methoxyethyl(methyl)aminomethyl; methyl; methyl-2,3,3a,4,6,6a-hexahydropyrrolo[2,3-c]pyrrolylmethyl; morpholinyl; morpholinylmethyl or pyrrolidinylmethyl;
R² is (dimethylamino)methyl(difluoro)pyrrolidinyl; (dimethylamino)methyl(fluoro)pyrrolidinyl; dimethylamino)methylpyrrolidinyl; cyano; cyclopropyl; cyclopropyl(methyl)aminomethylpyrrolidinyl; difluoroethyl(methyl)aminomethylpyrrolidinyl; difluoropyrrolidinyl; dimethylamino; dimethylaminopyrrolidinyl; hexahydropyrazino[2,1-c][1,4]oxazin-8(1H)-yl; methoxyethyl(methyl)amino; methoxyethyl(methyl)aminomethylpyrrolidinyl; methyl-2,3,3a,4,6,6a-hexahydropyrrolo[2,3-c]pyrrolyl; morpholinyl; phenylpyrrolidinyl; pyrrolidinyl; trifluoroethyl(methyl)amino or trifluoromethylpyrazolyl;
R³ is H, chloro or fluoro;
R⁴ is H, chloro or fluoro;
R⁵ is H or fluoro;
R⁶ is methyl or ethyl;
R⁷ is carboxy;
   or pharmaceutically acceptable salt thereof.

A further embodiment of present invention is (iii) a compound of formula (I) according to (i), or pharmaceutically acceptable salt thereof, wherein R¹ is H.

A further embodiment of present invention is (iv) a compound of formula (I) according to (i) or (iii), or pharmaceutically acceptable salt thereof, wherein R² is C₁₋₆alkyl-2,3,3a,4,6,6a-hexahydropyrrolo[2,3-c]pyrrolyl or (C₁₋₆alkyl)₂amino.

A further embodiment of present invention is (v) a compound of formula (I) according to (iv), or pharmaceutically acceptable salt thereof, wherein R² is methyl-2,3,3a,4,6,6a-hexahydropyrrolo[2,3-c]pyrrolyl or dimethylamino.

A further embodiment of present invention is (vi) a compound of formula (I) according to (i), wherein

R¹ is H;

R² is C₁₋₆alkyl-2,3,3a,4,6,6a-hexahydropyrrolo[2,3-c]pyrrolyl or (C₁₋₆alkyl)₂amino;

R³ is H or halogen;

R⁴ is H or halogen;

R⁵ is H or halogen;

R⁶ is C₁₋₆alkyl;

R⁷ is carboxy;

or pharmaceutically acceptable salt thereof.

A further embodiment of present invention is (vii) a compound of formula (I) according to (vi), wherein

R¹ is H;

R² is methyl-2,3,3a,4,6,6a-hexahydropyrrolo[2,3-c]pyrrolyl or dimethylamino;

R³ is H, chloro or fluoro;

R⁴ is H, chloro or fluoro;

R⁵ is H or fluoro;

R⁶ is methyl;

R⁷ is carboxy;

or pharmaceutically acceptable salt thereof.

Another embodiment of present invention is that (viii) compounds of formula (I) are selected from:
7-[4-(dimethylamino)-5,6-difluoro-8-(methylamino)-9H-pyrido[2,3-b]indol-3-yl]-4-oxo-pyrido[1,2-a]pyrimidine-3-carboxylic acid;
7-[4-(dimethylamino)-5,6-difluoro-8-(methylamino)-9H-pyrido[2,3-b]indol-3-yl]-9-methyl-4-oxo-pyrido[1,2-a]pyrimidine-3-carboxylic acid;
7-[4-(dimethylamino)-5,6-difluoro-8-(methylamino)-9H-pyrido[2,3-b]indol-3-yl]-9-methoxy-4-oxo-pyrido[1,2-a]pyrimidine-3-carboxylic acid;
7-[4-(dimethylamino)-6-fluoro-8-(methylamino)-9H-pyrido[2,3-b]indol-3-yl]-4-oxo-pyrido[1,2-a]pyrimidine-3-carboxylic acid;
7-[4-(dimethylamino)-6-fluoro-8-(methylamino)-9H-pyrido[2,3-b]indol-3-yl]-9-methyl-4-oxo-pyrido[1,2-a]pyrimidine-3-carboxylic acid;
7-[4-(dimethylamino)-5,6-difluoro-8-(methylamino)-9H-pyrido[2,3-b]indol-3-yl]-9-morpholino-4-oxo-pyrido[1,2-a]pyrimidine-3-carboxylic acid;
7-[5,6-difluoro-8-(methylamino)-4-pyrrolidin-1-yl-9H-pyrido[2,3-b]indol-3-yl]-9-morpholino-4-oxo-pyrido[1,2-a]pyrimidine-3-carboxylic acid;
7-[5,6-difluoro-4-(5-methyl-2,3,3a,4,6,6a-hexahydropyrrolo[2,3-c]pyrrol-1-yl)-8-(methylamino)-9H-pyrido[2,3-b]indol-3-yl]-4-oxo-pyrido[1,2-a]pyrimidine-3-carboxylic acid;
7-(5,6-difluoro-4-(methyl(2,2,2-trifluoroethyl)amino)-8-(methylamino)-9H-pyrido[2,3-b]indol-3-yl)-4-oxo-4H-pyrido[1,2-a]pyrimidine-3-carboxylic acid;
7-(5,6-difluoro-8-(methylamino)-4-morpholino-9H-pyrido[2,3-b]indol-3-yl)-4-oxo-4H-pyrido[1,2-a]pyrimidine-3-carboxylic acid;
7-(6-fluoro-8-(methylamino)-4-morpholino-9H-pyrido[2,3-b]indol-3-yl)-4-oxo-4H-pyrido[1,2-a]pyrimidine-3-carboxylic acid;
7-(4-(dimethylamino)-6,7-difluoro-8-(methylamino)-9H-pyrido[2,3-b]indol-3-yl)-4-oxo-4H-pyrido[1,2-a]pyrimidine-3-carboxylic acid;
7-(6,7-difluoro-8-(methylamino)-4-morpholino-9H-pyrido[2,3-b]indol-3-yl)-4-oxo-4H-pyrido[1,2-a]pyrimidine-3-carboxylic acid;
7-(5,6-difluoro-8-(methylamino)-4-morpholino-9H-pyrido[2,3-b]indol-3-yl)-9-((dimethylamino)methyl)-4-oxo-4H-pyrido[1,2-a]pyrimidine-3-carboxylic acid;
7-(5,6-difluoro-8-(methylamino)-4-(pyrrolidin-1-yl)-9H-pyrido[2,3-b]indol-3-yl)-9-((dimethylamino)methyl)-4-oxo-4H-pyrido[1,2-a]pyrimidine-3-carboxylic acid;
7-(6-chloro-4-(dimethylamino)-8-(methylamino)-9H-pyrido[2,3-b]indol-3-yl)-4-oxo-4H-pyrido[1,2-a]pyrimidine-3-carboxylic acid;
7-(4-(dimethylamino)-5,7-difluoro-8-(methylamino)-9H-pyrido[2,3-b]indol-3-yl)-4-oxo-4H-pyrido[1,2-a]pyrimidine-3-carboxylic acid;
7-(4-(3-((dimethylamino)methyl)-3-fluoropyrrolidin-1-yl)-5,6-difluoro-8-(methylamino)-9H-pyrido[2,3-b]indol-3-yl)-4-oxo-4H-pyrido[1,2-a]pyrimidine-3-carboxylic acid;
7-(5,6-difluoro-4-(3-(((2-methoxyethyl)(methyl)amino)methyl)pyrrolidin-1-yl)-8-(methylamino)-9H-pyrido[2,3-b]indol-3-yl)-4-oxo-4H-pyrido[1,2-a]pyrimidine-3-carboxylic acid;
7-(4-(3-4(2,2-difluoroethyl)(methyl)amino)methyl)pyrrolidin-1-yl)-5,6-difluoro-8-(methylamino)-9H-pyrido[2,3-b]indol-3-yl)-4-oxo-4H-pyrido[1,2-a]pyrimidine-3-carboxylic acid;
7-(4-(3-((cyclopropyl(methyl)amino)methyl)pyrrolidin-1-yl)-6-fluoro-8-(methylamino)-9H-pyrido[2,3-b]indol-3-yl)-4-oxo-4H-pyrido[1,2-a]pyrimidine-3-carboxylic acid;
7-[4-[4-[(dimethylamino)methyl]-3,3-difluoro-pyrrolidin-1-yl]-5,6-difluoro-8-(methylamino)-9H-pyrido[2,3-b]indol-3-yl]-4-oxo-pyrido[1,2-a]pyrimidine-3-carboxylic acid;
7-(5-chloro-4-(dimethylamino)-6-fluoro-8-(methylamino)-9H-pyrido[2,3-b]indol-3-yl)-4-oxo-4H-pyrido[1,2-a]pyrimidine-3-carboxylic acid;
7-(5,6-difluoro-4-(2-methoxyethyl)(methyl)amino)-8-(methylamino)-9H-pyrido[2,3-b]indol-3-yl)-4-oxo-4H-pyrido[1,2-a]pyrimidine-3-carboxylic acid;
7-(6-fluoro-8-(methylamino)-4-(3-phenylpyrrolidin-1-yl)-9H-pyrido[2,3-b]indol-3-yl)-4-oxo-4H-pyrido[1,2-a]pyrimidine-3-carboxylic acid;
(S)-7-(4-(2-((dimethylamino)methyl)pyrrolidin-1-yl)-6,7-difluoro-8-(methylamino)-9H-pyrido[2,3-b]indol-3-yl)-4-oxo-4H-pyrido[1,2-a]pyrimidine-3-carboxylic acid;
(R)-7-(4-(3-(dimethylamino)pyrrolidin-1-yl)-6,7-difluoro-8-(methylamino)-9H-pyrido[2,3-b]indol-3-yl)-4-oxo-4H-pyrido[1,2-a]pyrimidine-3-carboxylic acid;
7-(5,6-difluoro-4-(hexahydropyrazino[2,1-c][1,4]oxazin-8(1H)-yl)-8-(methylamino)-9H-pyrido[2,3-b]indol-3-yl)-4-oxo-4H-pyrido[1,2-a]pyrimidine-3-carboxylic acid compound with formic acid;
7-[8-(ethylamino)-6-fluoro-4-[3-(trifluoromethyl)pyrazol-1-yl]-9H-pyrido[2,3-b]indol-3-yl]-9-methyl-4-oxo-pyrido[1,2-a]pyrimidine-3-carboxylic acid;
7-[8-(ethylamino)-6-fluoro-4-[3-(trifluoromethyl)pyrazol-1-yl]-9H-pyrido[2,3-b]indol-3-yl]-4-oxo-pyrido[1,2-a]pyrimidine-3-carboxylic acid;
8-[8-(ethylamino)-6-fluoro-4-[3-(trifluoromethyl)pyrazol-1-yl]-9H-pyrido[2,3-b]indol-3-yl]-4-oxo-pyrido[1,2-a]pyrimidine-3-carboxylic acid;
7-[4-(dimethylamino)-5,6-difluoro-8-(methylamino)-9H-pyrido[2,3-b]indol-3-yl]-9-morpholino-4-oxo-pyrido[1,2-a]pyrimidine-3-carboxylic acid;
7-[5,6-difluoro-4-(5-methyl-2,3,3a,4,6,6a-hexahydropyrrolo[2,3-c]pyrrol-1-yl)-8-(methylamino)-9H-pyrido[2,3-b]indol-3-yl]-9-methyl-4-oxo-pyrido[1,2-a]pyrimidine-3-carboxylic acid;
7-[4-cyano-6-fluoro-8-(methylamino)-9H-pyrido[2,3-b]indol-3-yl]-4-oxo-pyrido[1,2-a]pyrimidine-3-carboxylic acid;
7-[4-(dimethylamino)-5,6-difluoro-8-(methylamino)-9H-pyrido[2,3-b]indol-3-yl]-9-(morpholinomethyl)-4-oxo-pyrido[1,2-a]pyrimidine-3-carboxylic acid;
7-[4-(dimethylamino)-5,6-difluoro-8-(methylamino)-9H-pyrido[2,3-b]indol-3-yl]-9-[[2-methoxyethyl(methyl)amino]methyl]-4-oxo-pyrido[1,2-a]pyrimidine-3-carboxylic acid;
7-[4-(dimethylamino)-5,6-difluoro-8-(methylamino)-9H-pyrido[2,3-b]indol-3-yl]-4-oxo-9-(pyrrolidin-1-ylmethyl)pyrido[1,2-a]pyrimidine-3-carboxylic acid;
7-[5,6-difluoro-8-(methylamino)-4-pyrrolidin-1-yl-9H-pyrido[2,3-b]indol-3-yl]-9-(morpholinomethyl)-4-oxo-pyrido[1,2-a]pyrimidine-3-carboxylic acid;
7-(4-(3,3-difluoropyrrolidin-1-yl)-6-fluoro-8-(methylamino)-9H-pyrido[2,3-b]indol-3-yl)-4-oxo-4H-pyrido[1,2-a]pyrimidine-3-carboxylic acid;
7-[4-(dimethylamino)-5,6-difluoro-8-(methylamino)-9H-pyrido[2,3-b]indol-3-yl]-9-[(5-methyl-2,3,3a,4,6,6a-hexahydropyrrolo[2,3-c]pyrrol-1-yl)methyl]-4-oxo-pyrido[1,2-a]pyrimidine-3-carboxylic acid; and
7-[4-cyclopropyl-5,6-difluoro-8-(methylamino)-9H-pyrido[2,3-b]indol-3-yl]-9-methyl-4-oxo-pyrido[1,2-a]pyrimidine-3-carboxylic acid;
   or pharmaceutically acceptable salt thereof.

### Synthesis

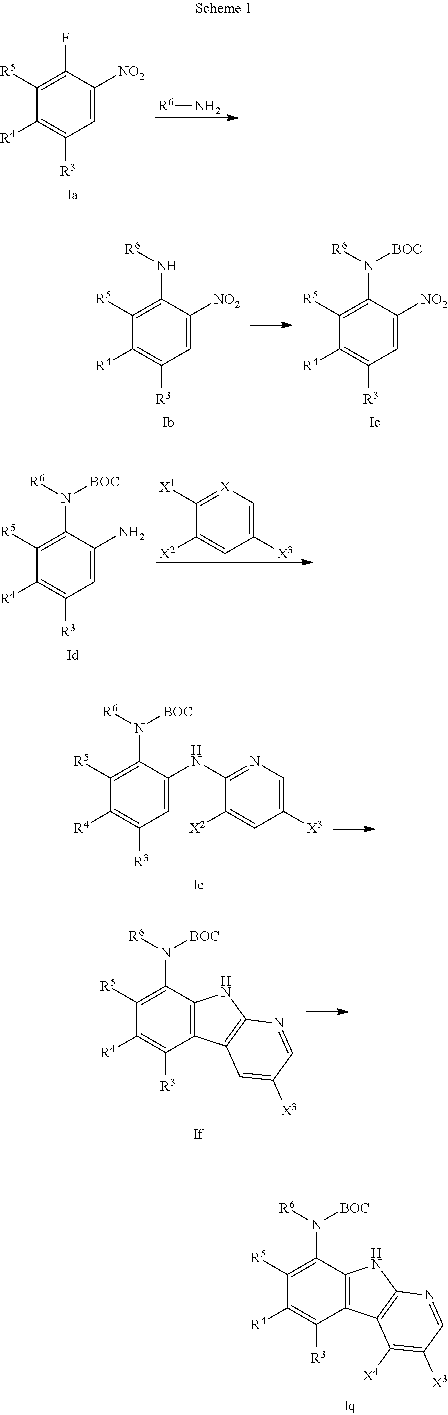

X¹, X², X³ and X⁴ are halogen.

Compound of formula (Ig) can be prepared according to Scheme 1. Nucleophilic substitution of ortho-fluoro nitrobenzene (Ia) with amine R⁶—NH₂ affords aniline (Ib). The aniline (Ib) can be protected with di-tert-butyl carbonate to give the protected aniline (Ic). The nitro group in aniline (Ic) can be reduced by a reducing agent, such as H₂ with palladium catalysts, to give the compound of formula (Id). Coupling of the compound of formula (Id) with tri-halogenated pyridine can be achieved using palladium catalysts and phosphine ligands to give compound of formula (Ie). Cyclization of compound of formula (Ie) using palladium catalysts and phosphine ligands gives compound of formula (If). Compound of formula (Ig) can be obtained from formula (If) through oxidation of the pyridine moiety of the tricyclic core followed by halogenation, such as treatment of POCl₃ or POBr₃.

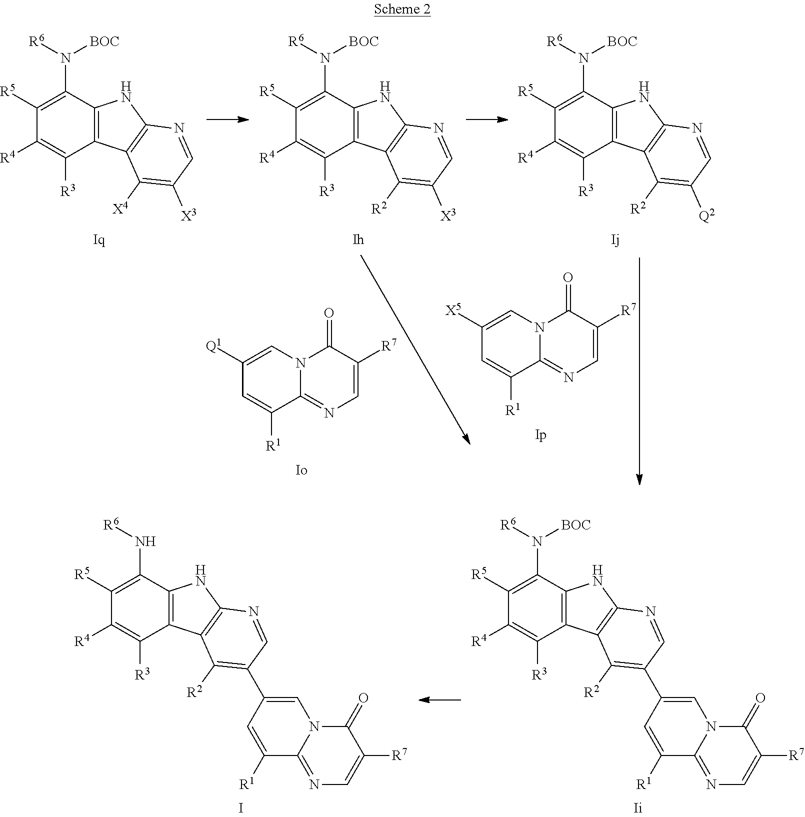

wherein X³ and X⁴ are halogen; X⁵ are halogen or OTf; Q¹ and Q² are boronic acids or esters.

Compound of formula (I) can be prepared according to Scheme 2. Introducing R² to compound of formula (Ig) can be achieved either through nucleophilic substitution with amine and a base for certain C—N bond formation (with R² bearing a nucleophilic N), or a Buchwald-Hartwig Cross Coupling Reaction for certain C—N bond formation (with R² bearing a basic N), to give compound of formula (Ih). Further coupling of compound of formula (Ih) with compound of formula (Io) to give compound of formula (Ii) can be achieved using a palladium catalyzed Suzuki coupling. Chiral separation can be achieved on compound of formula (Ih) or compound of formula (Ii). Some special compounds of formula (Ii) need to reverse the Suzuki coupling for C—C bond formation by converting compound of formula (Ih) to compound of formula (Ij) as a boronic ester or boronic acid then coupling with compound of formula (Ip). Ester hydrolysis such as NaOH in ethanol followed by deprotection of compound of formula (Ii) in the presence of an acid, such as trifluoroacetic acid, then affords compound of formula (I).

This invention also relates to a process for the preparation of a compound of formula (I) comprising the reaction of compound of formula (Ii),

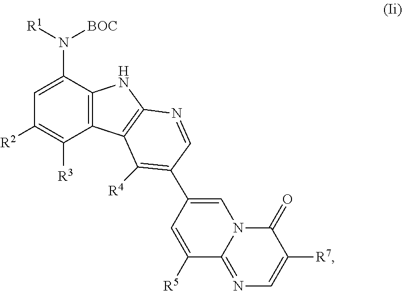

with an acid, which can be for example trifluoroacetic acid;

wherein R¹ to R⁷ are defined above. A compound of formula (I) when manufactured according to the above process is also an object of the invention.

### Pharmaceutical Compositions and Administration

Another embodiment provides pharmaceutical compositions or medicaments containing the compounds of the invention and a therapeutically inert carrier, diluent or excipient, as well as methods of using the compounds of the invention to prepare such compositions and medicaments. In one example, compounds of formula (I) may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula (I) is formulated in an acetate buffer, at pH 5. In another embodiment, the compounds of formula (I) are sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “effective amount” of the compound to be administered will be governed by such considerations, and is the minimum amount necessary to reduced bacterial load or improve host survival through the inhibition of bacterial DNA gyrase and/or Topoisomerase IV. For example, such amount may be below the amount that is toxic to normal cells, or the mammal as a whole.

In one example, the pharmaceutically effective amount of the compound of the invention administered parenterally per dose will be in the range of about 0.1 to 1000 mg/kg, alternatively about 1 to 100 mg/kg of patient body weight per day. In another embodiment, oral unit dosage forms, such as tablets and capsules, preferably contain from about 5 to about 5000 mg of the compound of the invention.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

An example of a suitable oral dosage form is a tablet containing about 10 to 500 mg of the compound of the invention compounded with about 40 to 400 mg anhydrous lactose, about 5 to 50 mg sodium croscarmellose, about 5 to 50 mg polyvinylpyrrolidone (PVP) K30, and about 1 to 10 mg magnesium stearate. The powdered ingredients are first mixed together and then mixed with a solution of the PVP. The resulting composition can be dried, granulated, mixed with the magnesium stearate and compressed to tablet form using conventional equipment. An example of an aerosol formulation can be prepared by dissolving the compound, for example 5 to 1000 mg) of the invention in a suitable buffer solution, e.g. a phosphate buffer, adding a tonicifier, e.g. a salt such sodium chloride, if desired. The solution may be filtered, e.g., using a 0.2 micron filter, to remove impurities and contaminants.

An embodiment, therefore, includes a pharmaceutical composition comprising a compound of formula (I), or a stereoisomer or pharmaceutically acceptable salt thereof. In a further embodiment includes a pharmaceutical composition comprising a compound of formula (I), or a stereoisomer or pharmaceutically acceptable salt thereof, together with a pharmaceutically acceptable carrier or excipient.

Another embodiment includes a pharmaceutical composition comprising a compound of formula (I) for use in the treatment and/or prophylaxis of bacterial infections.

In some embodiments, the compounds of this invention may be administered, as part of a single or multiple dosage regimen, orally, parenterally, by inhalation spray, topically, rectally, nasally, buccally, vaginally, or via an implanted reservoir. The term parenteral as used includes subcutaneous, intracutaneous, intravenous, intramuscular, intra-articular, intrasynovial, intrasternal, intrathecal, intralesional and intracranial injection or infusion techniques. Typically, the pharmaceutical compositions of the invention will be administered from about 1 to 5 times per day or alternatively, as a continuous infusion upon improvement of a patient's condition.

### Indications and Methods of Treatment

The compounds of the invention are useful for treatment and/or prophylaxis of bacterial infection in humans or other animals by administering to the subject in need of a therapeutically effective amount of compound of formula (I), or a pharmaceutically acceptable salt, or enantiomer or diastereomer thereof. The compounds and methods of the invention are particularly well suited for human patients infected by pathogens that include Staphylococcus aureus, Escherichia coli, Klebsiella pneumoniae, Acinetobacter baumannii and Pseudomonas aeruginosa. Examples of bacterial organisms that may also be controlled by the compounds of the invention include, but not limited to, the following Gram-Positive and Gram-Negative organisms: Streptococcus pneumoniae, Streptococcus pyogenes, Enterococcus faecalis, Enterococcus faecium, Enterobacter spp. species, Proteus spp. species, Serratia marcescens, Staphylococcus aureus, Coag. Neg. Staphylococci, Haemophilus influenzae, Bacillus anthraces, Mycoplasma pneumoniae, Moraxella catarrhalis, Chlamydophila pneumoniae, Chlamydia trachomatis, Legionella pneumophila, Mycobacterium tuberculosis, Helicobacter pylori, Staphylococcus saprophyticus, Staphylococcus epidermidis, Francisella tularensis, Yersinia pestis, Clostridium difficile, Bacteroides spp. species Neisseria gonorrhoeae, Neisseria meningitidis, Burkholderia pseudomallei, Burkholderia mallei, Borrelia burgdorferi, Mycobacterium avium complex, Mycobacterium abscessus, Mycobacterium kansasii and Mycobacterium ulcerans.

Examples of bacterial infections may include, but not limited to, upper respiratory infections, lower respiratory infections, ear infections, pleuropulmonary and bronchial infections, complicated urinary tract infections, uncomplicated urinary tract infections, intra-abdominal infections, cardiovascular infections, a blood stream infection, sepsis, bacteremia, CNS infections, skin and soft tissue infections, GI infections, bone and joint infections, genital infections, eye infections, or granulomatous infections. Examples of specific bacterial infections include, but not limited to, uncomplicated skin and skin structure infections (uSSSI), complicated skin and skin structure infections (cSSSI), catheter infections, pharyngitis, sinusitis, otitis extema, otitis media, bronchitis, empyema, pneumonia, community-acquired bacterial pneumoniae (CABP), hospital-acquired pneumonia (HAP), hospital-acquired bacterial pneumonia, ventilator-associated pneumonia (VAP), diabetic foot infections, vancomycin resistant enterococci infections, cystitis and pyelonephritis, renal calculi, prostatitis, peritonitis, complicated intra-abdominal infections (cIAI) and other inter-abdominal infections, dialysis-associated peritonitis, visceral abscesses, endocarditis, myocarditis, pericarditis, transfusion-associated sepsis, meningitis, encephalitis, brain abscess, osteomyelitis, arthritis, genital ulcers, urethritis, vaginitis, cervicitis, gingivitis, conjunctivitis, keratitis, endophthalmitisa, an infection in cystic fibrosis patients or an infection of febrile neutropenic patients.

Furthermore, the invention relates to the use of a compound of formula (I) for the treatment and/or prophylaxis of bacterial infection. The invention relates to the use of a compound of formula (I) for the preparation of a medicament for the treatment and/or prophylaxis of bacterial infection. Another embodiment includes a method for the treatment or prophylaxis of bacterial infection which method comprises administering an effective amount of a compound of formula (I), or pharmaceutically acceptable salt, or enantiomer or diastereomer thereof.

### EXAMPLES

The invention will be more fully understood by reference to the following examples. They should not, however, be construed as limiting the scope of the invention.

### Abbreviations

Abbreviations used herein are as follows:
AcOK potassium acetate
B₂Pin₂ Bis(pinacolato)diboron
BINAP 2,2′-Bis(diphenylphosphino)-1,1′-binaphthalene
CAMHB Caution-Adjusted Mueller Hinton Broth
CC₅₀ concentration results in the death of 50 percent of the cells
DBU 1,8-Diazabicyclo[5.4.0]undec-7-ene
DCM dichloromethane
DIPEA N,N-diisopropylethylamine
EtOAc ethyl acetate
h hour
HATU: 1-[Bis(dimethylamino)methylene]-1H-1,2,3-triazolo[4,5-b]pyridinium 3-oxid hexafluorophosphate
HPLC: high performance liquid chromatography
HPLC-UV: high performance liquid chromatography with ultraviolet detector
MIC minimum inhibitory concentration
Pd-Ad₂nBuP Biphenyl Chloro[(di(1-adamantyl)-N-butylphosphine)-2-(2-aminobiphenyl)]palladium(II)
Precat precatalyst
prep-HPLC preparative high performance liquid chromatography
rt room temperature
SFC supercritical fluid chromatography
SM start material
TLC Thin Layer Chromatography
UV ultraviolet detector

### General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel Brand and pore size: i) KP-SIL 60 Å, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using X Bridge™ Perp C₁₈ (5 μm, OBD™ 30×100 mm) column or SunFire™ Perp C₁₈ (5 OBD™ 30×100 mm) column.

Chiral Separation was conducted on Thar 350 preparative SFC using ChiralPak AD-10μ (200×50 mm I.D.) with mobile phase A for CO₂ and B for ethanol. LC/MS spectra were obtained using a Waters UPLC-SQD Mass. Standard LC/MS conditions were as follows (running time: 3 minutes):

Acidic condition: A: 0.1% formic acid and 1% acetonitrile in H₂O; B: 0.1% formic acid in acetonitrile;

Basic condition: A: 0.05% NH₃.H₂O in H₂O; B: acetonitrile.

Mass spectra (MS): generally only ions which indicate the parent mass are reported, and unless otherwise stated the mass ion quoted is the positive mass ion (M+H)⁺.

NMR Spectra were obtained using Bruker Avance 400 MHz.

All reactions involving air-sensitive reagents were performed under an argon atmosphere. Reagents were used as received from commercial suppliers without further purification unless otherwise noted.

### Preparative Examples

### Intermediate A1

### Tert-Butyl N-(3,4-dichloro-6-fluoro-9H-pyrido[2,3-b]indol-8-yl)-N-methyl-carbamate

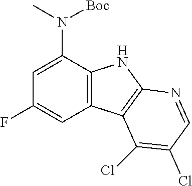 [SMILES: CN(C(=O)OC(C)(C)C)c1cc(F)cc2c1[nH]c1ncc(Cl)c(Cl)c12]

The titled compound was synthesized according to the following scheme:

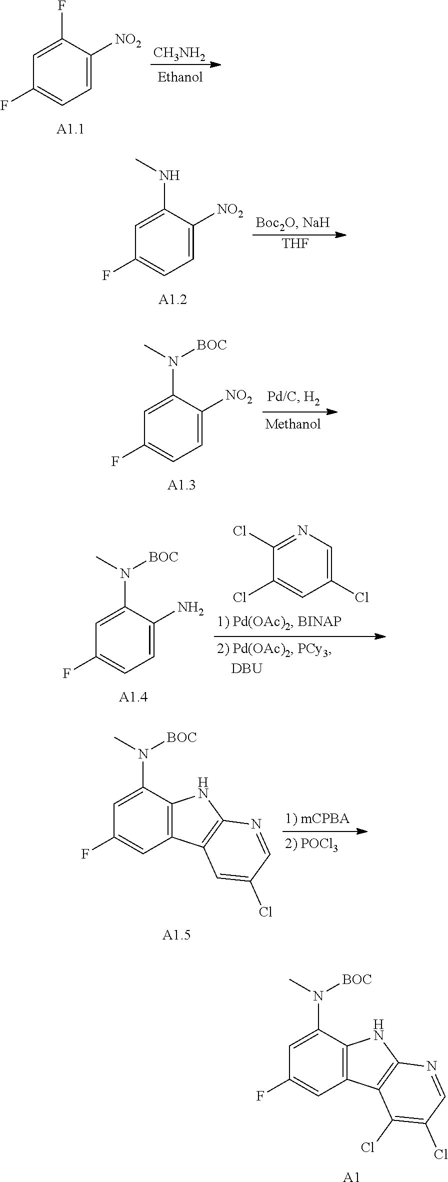

### Step (a) Preparation of 5-fluoro-N-methyl-2-nitro-aniline (Compound A1.2)

Methylamine solution (355 g, 2.866 mol, 25% in EtOH) was added dropwise to 2,4-difluoronitrobenzene (147 g, 0.924 mol) at 0° C. over 15 min. After completion of the addition, the reaction mixture was stirred at 0° C. for 2 h. The solution was diluted with ethanol (500 mL) and poured into 2 L of ice-water. The resulting precipitates were collected by filtration and dried in vacuo to give 5-fluoro-N-methyl-2-nitro-aniline (143 g, 63% yield) as a yellow solid.

### Step (b) Preparation of Tert-Butyl N-(5-fluoro-2-nitro-phenyl)-N-methyl-carbamate (Compound A1.3)

To a suspension of sodium hydride (141 g, 3.5 mol, 60% dispersion in mineral oil) in dry THF (2 L) was added 5-fluoro-N-methyl-2-nitro-aniline (60 g, 0.35 mol, compound A1.2) portion wise at 0° C. After the solution was stirred at 0° C. for 1 h, a solution of di-tert-butyl dicarbonate (115 g, 0.53 mol) in THF (0.5 L) was added dropwise and the reaction mixture continued stirring for another 15 h at 15° C. The reaction mixture was poured into 1.6 L of ice-water and extracted with EtOAc (1.6 L) two times. The combined organics were dried over anhy. sodium sulfate and concentrated in vacuo. The crude product was purified by silica gel flash chromatography (1-5% EtOAc in petroleum ether) to give tert-butyl N-(5-fluoro-2-nitro-phenyl)-N-methyl-carbamate (70 g, 73% yield) as a yellow solid. MS (ESI): 293.0 ([M+Na]⁺), 171.0 ([M-C₄H₈—CO₂+H]⁺).

### Step (c) Preparation of Tert-Butyl N-(2-amino-5-fluoro-phenyl)-N-methyl-carbamate (Compound A1.4)

To a solution of tert-butyl N-(5-fluoro-2-nitro-phenyl)-N-methyl-carbamate (70 g, 259 mmol, compound A1.3) in MeOH (1 L) was added palladium on carbon (5 g, 10 wt. % loading). The reaction mixture was stirred at 16° C. for 18 h under H₂ atmosphere (50 psi). After the remaining palladium catalyst was removed by filtration, the filtrate was concentrated in vacuo to give tert-butyl N-(2-amino-5-fluoro-phenyl)-N-methyl-carbamate (60 g, 96% yield) as a white solid. MS (ESI): 263.1 ([M+Na]⁺), 185.0 ([M-C₄H₈+H]⁺), 141.0 ([M-C₄H₈—CO₂+H]⁺).

### Step (d) Preparation of Tert-Butyl N-(3-chloro-6-fluoro-9H-pyrido[2,3-b]indol-8-yl)-N-methyl-carbamate (Compound A1.5)

To a solution of tert-butyl N-(2-amino-5-fluoro-phenyl)-N-methyl-carbamate (80 g, 333 mmol, compound A1.4) and 2,3,5-trichloropyridine (66.8 g, 366 mmol, CAS: 16063-70-0) in dioxane (2 L) were added cesium carbonate (217 g, 666 mmol), palladium(II) acetate (3.74 g, 16.7 mmol), and BINAP (20.7 g, 33.3 mmol, CAS: 98327-87-8). The reaction mixture was stirred at 120° C. for 16 h under nitrogen atmosphere. After the reaction mixture was cooled back to room temperature, it was diluted with EtOAc (800 mL). The precipitate was removed by filtration and the filtrate was concentrated in vacuo, and the crude product was purified by silica gel flash chromatography (0.2% to 5% EtOAc in petroleum ether) to give intermediate tert-butyl N-[2-[(3,5-dichloro-2-pyridyl)amino]-5-fluoro-phenyl]-N-methyl-carbamate (75 g, 58% yield) as a white solid. MS (ESI): 390.1 ([{³⁷Cl}M+H]⁺), 388.1 ([{³⁷Cl+³⁵Cl} M+H]⁺), 386.1 ([{³⁵Cl}M+H]⁺).

Next, to a solution of tert-butyl N-[2-[(3,5-dichloro-2-pyridyl)amino]-5-fluoro-phenyl]-N-methyl-carbamate (5 g, 12.95 mmol) and DBU (3.94 g, 25.9 mmol, CAS: 6674-22-2) in the mixture of o-xylene (7.5 mL) and N,N-dimethylacetamide (7.5 mL) were added palladium(II) acetate (727 mg, 3.24 mmol) and tricyclohexylphosphine tetrafluoroborate (2.38 g, 6.48 mmol) under nitrogen atmosphere. The reaction mixture was stirred at 160° C. for 6 h before it was cooled back to room temperature and poured into water (100 mL). The mixture was extracted with EtOAc (200 mL) and the organic layer was collected and washed with water (50 mL) two times, brine (30 mL) two times, and dried over anhy. sodium sulfate. The separated organic layer was concentrated in vacuo to give a crude product, which was purified by silica gel flash chromatography (0.5% to 20% EtOAc in DCM) to give tert-butyl N-(3-chloro-6-fluoro-9H-pyrido[2,3-b]indol-8-yl)-N-methyl-carbamate (873 mg, 19.3% yield) as a yellow solid. MS (ESI): 352.1 ([{³⁷Cl} M+H]⁺), 350.1 ([{³⁵Cl} M+H]⁺).

### Step (e) Preparation of Tert-Butyl N-(3,4-dichloro-6-fluoro-9H-pyrido[2,3-b]indol-8-yl)-N-methyl-carbamate (Intermediate A1)

To a solution of tert-butyl N-(3-chloro-6-fluoro-9H-pyrido[2,3-b]indol-8-yl)-N-methyl-carbamate (4.8 g, 13.7 mmol, compound A1.5) in DCM (200 mL) was added 3-chloroperbenzoic acid (9.47 g, 54.9 mmol) at 0° C. under nitrogen atmosphere. Afterwards, the reaction mixture was allowed to warm up to 30° C. and stirred for 12 h. Then the reaction mixture was cooled back to room temperature and poured into aq. sodium sulfite solution (10%, 150 mL) and stirred for 1 h followed by extraction with EtOAc (750 mL) three times. The combined organics were washed by aq. sodium bicarbonate solution (5N, 200 mL), brine (250 mL), dried over anhy. sodium sulfate, and concentrated in vacuo to give a crude product of tert-butyl N-(3-chloro-6-fluoro-1-oxido-9H-pyrido [2,3-b]indol-1-ium-8-yl)-N-methyl-carbamate (4.8 g, 96% yield) as a brown solid. MS (ESI): 368.1 ([{³⁷Cl} M+H]⁺), 366.1 ([{³⁵Cl} M+H]⁺).

Next, to a solution of tert-butyl N-(3-chloro-6-fluoro-1-oxido-9H-pyrido[2,3-b] indol-1-ium-8-yl)-N-methyl-carbamate (4.8 g, 13.1 mmol) in DMF (100 mL) was added phosphorus(V) oxychloride (22.1 g, 144 mmol) dropwise at −5° C. The mixture was stirred at −5° C. to 0° C. for 1 h before poured into satd. aq. sodium bicarbonate solution (350 mL) at 0° C. The mixture was then extracted by EtOAc (500 mL) three times and the combined organics were washed with water (200 mL) three times, brine (150 mL) two times, dried over anhy. sodium sulfate, and concentrated in vacuo. The crude product was then purified by washing with MeOH (120 mL) to give tert-butyl N-(3,4-dichloro-6-fluoro-9H-pyrido[2,3-b]indol-8-yl)-N-methyl-carbamate (2.16 g, 42.9% yield) as a pale yellow solid. MS (ESI): 388.1 ([{³⁷Cl+³⁷Cl} M+H]⁺), 386.1 ([{³⁷Cl+³⁵Cl} M+H]⁺), 384.1 ([{³⁵Cl+³⁵Cl} M+H]⁺). ¹H NMR (400 MHz, DMSO-d6) δ ppm: 12.55 (s, 1H), 8.65 (s, 1H), 8.05 (d, J=7.0 Hz, 1H) 7.49 (dd, J=10.3, 2.5 Hz, 1H) 3.26 (s, 3H) 1.19-1.62 (m, 9H).

### Intermediate A2

### Tert-Butyl N-methyl-N-(3,4,6-trichloro-911-pyrido[2,3-b]indol-8-yl)carbamate

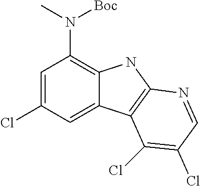 [SMILES: CN(C(=O)OC(C)(C)C)c1cc(Cl)cc2c1[nH]c1ncc(Cl)c(Cl)c12]

In analogy to the synthesis of tert-butyl N-(3,4-dichloro-6-fluoro-9H-pyrido[2,3-b] indol-8-yl)-N-methyl-carbamate (Intermediate A1), the title compound was prepared by replacing 2,4-difluoronitrobenzene with 4-chloro-2-fluoro-1-nitrobenzene in step (a). MS (ESI): 445.9 ([M+H]⁺). ¹H NMR (400 MHz, DMSO-d6) δ 12.665 (s, 1H), 8.728 (s, 1H), 8.267 (s, 1H), 7.602 (s, 1H), 3.259 (s, 3H), 1.15-51.508 (m, 3H).

### Intermediate A3

### Tert-Butyl N-(3,4-dichloro-5,6-difluoro-911-pyrido[2,3-b]indol-8-yl)-N-methyl-carbamate

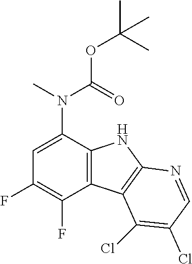 [SMILES: CN(C(=O)OC(C)(C)C)c1cc(F)c(F)c2c1[nH]c1ncc(Cl)c(Cl)c12]

In analogy to the synthesis of tert-butyl N-(3,4-dichloro-6-fluoro-9H-pyrido[2,3-b]indol-8-yl)-N-methyl-carbamate (Intermediate A1), the title compound was prepared by replacing 2,4-difluoronitrobenzene with 2,4,5-trifluoronitrobenzene in step (a). MS (ESI): 406.1 ([{³⁷Cl+³⁷Cl} M+H]⁺), 404.1 ([{³⁷Cl+³⁵Cl} M+H]⁺), 402.1 ([{³⁵Cl+³⁵Cl} M+H]⁺). ¹H NMR (400 MHz, DMSO-d6) δ ppm: 12.84 (br. s, 1H), 8.64 (s, 1H), 7.70 (m, 1H), 3.22 (s, 3H), 1.22-1.50 (m, 9H).

### Intermediate A4

### Tert-Butyl N-(3,4-dichloro-6,7-difluoro-9H-pyrido[2,3-b]indol-8-yl)-N-methyl-carbamate

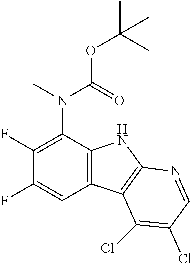 [SMILES: CN(C(=O)OC(C)(C)C)c1c(F)c(F)cc2c1[nH]c1ncc(Cl)c(Cl)c12]

In analogy to the synthesis of tert-butyl N-(3,4-dichloro-6-fluoro-9H-pyrido[2,3-b]indol-8-yl)-N-methyl-carbamate (Intermediate A1), the title compound was prepared by replacing 2,4-difluoronitrobenzene with 2,3,4-trifluoronitrobenzene in step (a). MS (ESI): 406.2 ([{³⁷Cl+³⁷Cl} M+H]⁺), 404.2 ([{³⁷Cl+³⁵Cl} M+H]⁺), 402.1 ([{³⁵Cl+³⁵Cl} M+H]⁺). ¹H NMR (400 MHz, DMSO-d6) δ ppm: 12.61 (br. s, 1H), 8.59 (s, 1H), 8.26 (m, 1H), 3.04 (s, 3H), 1.22-1.50 (m, 9H).

### Intermediate A5

### Tert-Butyl (3,4-dichloro-5,7-difluoro-9H-pyrido[2,3-b]indol-8-yl)(methyl)carbamate

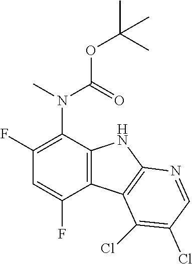 [SMILES: CN(C(=O)OC(C)(C)C)c1c(F)cc(F)c2c1[nH]c1ncc(Cl)c(Cl)c12]

In analogy to the synthesis of tert-butyl N-(3,4-dichloro-6-fluoro-9H-pyrido[2,3-b]indol-8-yl)-N-methyl-carbamate (Intermediate A1), the title compound was prepared by replacing 2,4-difluoro-1-nitrobenzene with 1,2,5-trifluoro-3-nitrobenzene in step (a). MS (ESI): 402.0 ([{³⁷Cl+³⁷Cl} M+H]⁺). ¹H NMR (400 MHz, DMSO-d6) δ ppm: 12.84 (br. s, 1H), 8.64 (s, 1H), 7.70 (m, 1H), 3.22 (s, 3H), 1.22-1.50 (m, 9H).

### Intermediate A6

### Tert-Butyl methyl(3,4,5-trichloro-6-fluoro-911-pyrido[2,3-b]indol-8-yl)carbamate

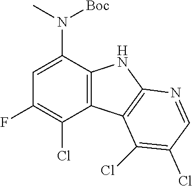 [SMILES: CN(C(=O)OC(C)(C)C)c1cc(F)c(Cl)c2c1[nH]c1ncc(Cl)c(Cl)c12]

The titled compound was synthesized according to the following scheme:

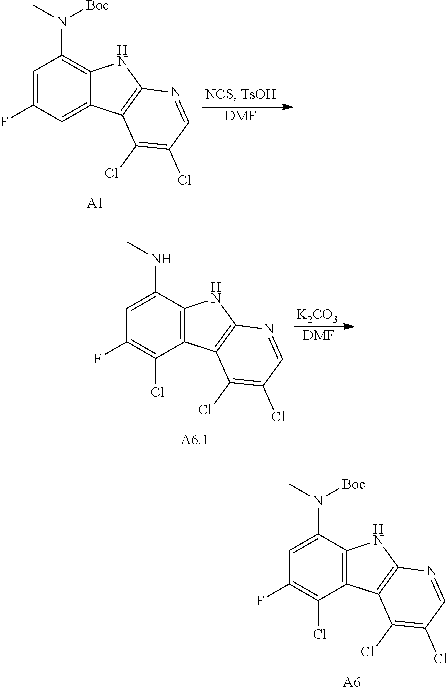

### Step (a) Preparation of 3,4,5-trichloro-6-fluoro-N-methyl-9H-pyrido[2,3-b]indol-8-amine (Compound A6.1)

To a stirred solution of tert-butyl (3,4-dichloro-6-fluoro-9H-pyrido[2,3-b]indol-8-yl)(methyl)carbamate (5 g, 13.02 mmol, Intermediate A1) in DMF (150 mL) was added 1-chloropyrrolidine-2,5-dione (2.27 g, 39.06 mmol) and 4-methylbenzenesulfonic acid (4.48 mg, 26.04 mmol). The mixture was stirred at 90° C. for 12 h until LC-MS showed the starting material was completely consumed. The reaction mixture was cooled back to r.t. and concentrated in vacuo. The crude product was purified by silica gel flash chromatography (petroleum ether:EtOAc=10:1˜1:1) to give 3,4,5-trichloro-6-fluoro-N-methyl-9H-pyrido[2,3-b]indol-8-amine (3 g, 71.9% yield) as a yellow solid. MS (ESI): 319.9 (M+H]⁺).

### Step (b) Preparation of Tert-Butyl methyl(3,4,5-trichloro-6-fluoro-9H-pyrido[2,3-b]indol-8-yl)carbamate (Intermediate A6)

To a stirred solution of 3,4,5-trichloro-6-fluoro-N-methyl-9H-pyrido[2,3-b]indol-8-amine (3 g, 9.416 mmol) and K₂CO₃ (6.5 g, 47.08 mmol) in DMF (60 mL) was added di-tert-butyl dicarbonate (6.2 g, 28.25 mmol). The mixture was stirred at 25° C. for 12 h until TLC (petroleum ether:EtOAc=10:1) and LC-MS showed the starting material was consumed completely. The reaction mixture was concentrated in vacuo to give a crude product, which was purified by silica gel flash chromatography (petroleum ether:EtOAc=20:1˜10:1) to give tert-butyl methyl(3,4,5-trichloro-6-fluoro-9H-pyrido[2,3-b]indol-8-yl)carbamate (2.1 g; 53.8% yield) as a yellow solid. MS (ESI): 418.0 ([{³⁵Cl} M+H]⁺), 317.9 {M−56}+H⁺). ¹H NMR (400 MHz, DMSO-d6) δ ppm 9.75 (s, 1H), 8.59 (s, 1H), 7.27-7.30 (m, 1H), 3.37 (s, 3H), 1.43 (m, 9H).

### Intermediate A7

### Tert-Butyl N-(3,4-dichloro-6,7-difluoro-911-pyrido[2,3-b]indol-8-yl)-N-methyl-carbamate

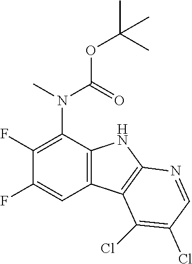 [SMILES: CN(C(=O)OC(C)(C)C)c1c(F)c(F)cc2c1[nH]c1ncc(Cl)c(Cl)c12]

In analogy to the synthesis of tert-butyl N-(3,4-dichloro-6-fluoro-9H-pyrido[2,3-b]indol-8-yl)-N-methyl-carbamate (Intermediate A1), the title compound was prepared by replacing 2,4-difluoronitrobenzene with 2,5,6-trifluoronitrobenzene in step (a). MS (ESI): 406.1 ([{³⁷Cl+³⁷Cl} M+H]⁺), 404.1 ([{³⁷Cl+³⁵Cl} M+H]⁺), 402.1 ([{³⁵Cl+³⁵Cl} M+H]⁺). ¹H NMR (400 MHz, DMSO-d6) δ ppm: 12.84 (br. s, 1H), 8.64 (s, 1H), 7.72 (m, 1H), 3.22 (s, 3H), 1.22-1.50 (m, 9H).

### Intermediate B1

### (3-Ethoxycarbonyl-4-oxo-pyrido[1,2-a]pyrimidin-7-yl)boronic Acid

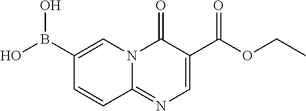 [SMILES: CCOC(=O)c1cnc2ccc(B(O)O)cn2c1=O]

The titled compound was synthesized according to the following scheme:

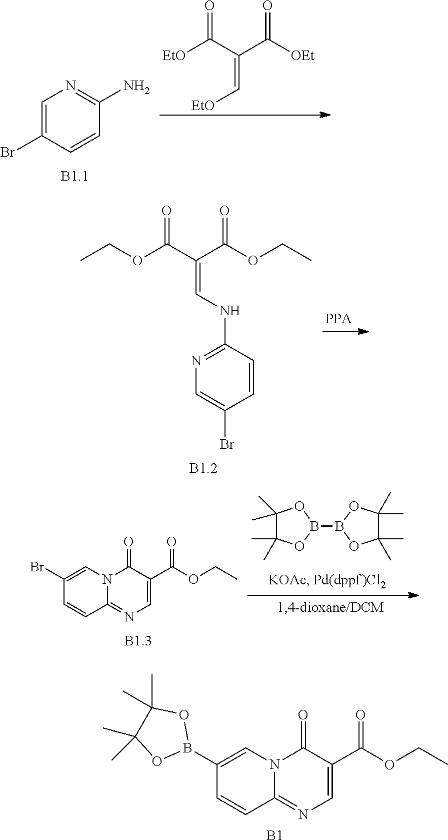

### Step (a) Preparation of Diethyl 2-[[(5-bromo-2-pyridyl)amino]methylene]propanedioate (Compound B1.2)

A mixture of 5-bromopyridin-2-amine (11.23 g, 64.9 mmol) and diethyl 2-(ethoxymethylene)malonate (14 g, 64.9 mmol) was stirred at 110° C. for 30 min. The reaction mixture was cooled back to r.t., and lots of white solids were formed in the meantime. The solid was collected through filtration and dried under vacuum. It was used directly in the next step without further purification. MS: 343.0 (Br⁷⁹[M+H]⁺), 345.1 (Br⁸¹ [M+H]⁺).

### Step (b) Preparation of Ethyl 7-bromo-4-oxo-pyrido[1,2-a]pyrimidine-3-carboxylate (Compound B1.3)

A mixture of diethyl 2-(((5-bromopyridin-2-yl)amino)methylene)malonate (22.3 g, 65 mmol) and PPA (65.9 g, 31.4 ml, 195 mmol, CAS: 8017-16-1) was stirred at 130° C. for 30 min. The reaction mixture was cooled back to r.t., poured into water (100 mL) and extracted with DCM (100 mL). The separated organic lay was dried over anhy. Na₂SO₄, filtered, and concentrated in vacuo to give a crude product, which was purified by silica gel flash chromatography (DCM:EtOAc=2:1) to give ethyl 7-bromo-4-oxo-pyrido[1,2-a]pyrimidine-3-carboxylate (6.72 g, 34.8% yield) as a white solid. MS: 297.0 ([M+H]⁺).

### Step (c) Preparation of (3-ethoxycarbonyl-4-oxo-pyrido[1,2-a]pyrimidin-7-yl)boronic Acid (Intermediate B1)

To a stirred solution of ethyl 7-bromo-4-oxo-pyrido[1,2-a]pyrimidine-3-carboxylate (401 mg, 1.35 mmol), 4,4,4′,4′,5,5,5′,5′-octamethyl-2,2′-bi(1,3,2-dioxaborolane) (514 mg, 2.02 mmol), and AcOK (397 mg, 4.05 mmol) in 1,4-dioxane (10 mL) was added Pd(dppf)Cl₂.CH₂Cl₂ (110 mg, 135 μmol) at room temperature. The resulting mixture was stirred at 90° C. for 6 h before it was cooled back to room temperature. The mixture was filtered and the filtrate was concentrated in vacuo to give a crude product, which was purified by silica gel flash chromatography to give (3-ethoxycarbonyl-4-oxo-pyrido[1,2-a]pyrimidin-7-yl)boronic acid (168 mg, 47.5% yield) as a white solid. MS: 263.1 ([M−41]⁺).

### Intermediate B2

### (3-Ethoxycarbonyl-9-methyl-4-oxo-pyrido[1,2-a]pyrimidin-7-yl)boronic Acid

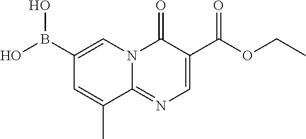 [SMILES: CCOC(=O)c1cnc2c(C)cc(B(O)O)cn2c1=O]

The titled compound was synthesized according to the following scheme:

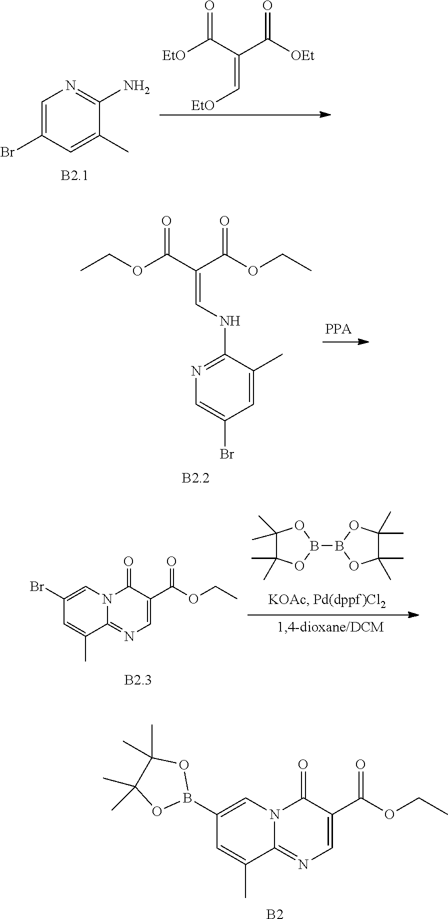

### Step (a) Preparation of Diethyl 2-(((5-bromo-3-methylpyridin-2-yl)amino)methylene)malonate (Compound B2.2)

In analogy to the synthesis of diethyl 2-[[(5-bromo-2-pyridyl)amino]methylene]propanedioate (compound B1.2), the title compound was prepared by replacing 5-bromopyridin-2-amine with 5-bromo-3-methylpyridin-2-amine. MS (ESI): 358.9 ([{⁸¹Br} M+H]⁺).

### Step (b) Preparation of Ethyl 7-bromo-9-methyl-4-oxo-4H-pyrido[1,2-a]pyrimidine-3-carboxylate (Compound B2.3)

In analogy to the synthesis of ethyl 7-bromo-4-oxo-pyrido[1,2-a]pyrimidine-3-carboxylate (compound B1.3), the title compound was prepared by replacing diethyl 2-(((5-bromopyridin-2-yl)amino)methylene)malonate with diethyl 2-(((5-bromo-3-methylpyridin-2-yl)amino)methylene)malonate. MS (ESI): 310.9 ([{⁸¹Br} M+H]⁺).

### Step (c) Preparation of (3-ethoxycarbonyl-9-methyl-4-oxo-pyrido[1,2-a]pyrimidin-7-yl)boronic Acid (Intermediate B2)

In analogy to the synthesis of (3-ethoxycarbonyl-4-oxo-pyrido[1,2-a]pyrimidin-7-yl)boronic acid (Intermediate B1), the title compound was prepared by replacing ethyl 7-bromo-4-oxo-pyrido[1,2-a]pyrimidine-3-carboxylate with ethyl 7-bromo-9-methyl-4-oxo-4H-pyrido[1,2-a]pyrimidine-3-carboxylate. MS (ESI): 277.0 ([M+H]⁺).

### Intermediate B3

### (3-Ethoxycarbonyl-9-methoxy-4-oxo-pyrido[1,2-a]pyrimidin-7-yl)boronic Acid

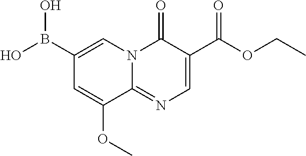 [SMILES: CCOC(=O)c1cnc2c(OC)cc(B(O)O)cn2c1=O]

In analogy to the synthesis of (3-ethoxycarbonyl-4-oxo-pyrido[1,2-a]pyrimidin-7-yl)boronic acid (Intermediate B1), the title compound was prepared by replacing 5-bromopyridin-2-amine with 5-bromo-3-methoxy-pyridin-2-amine in step (a). MS (ESI): 292.6 ([M+H]⁺).

### Intermediate B4

### (3-Ethoxycarbonyl-9-morpholino-4-oxo-pyrido[1,2-a]pyrimidin-7-yl)boronic Acid

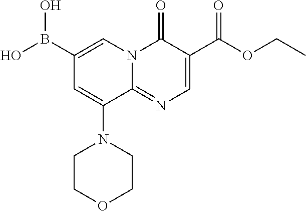 [SMILES: CCOC(=O)c1cnc2c(N3CCOCC3)cc(B(O)O)cn2c1=O]

In analogy to the synthesis of (3-ethoxycarbonyl-4-oxo-pyrido[1,2-a]pyrimidin-7-yl)boronic acid (Intermediate B1), the title compound was prepared by replacing 5-bromopyridin-2-amine with 5-bromo-3-morpholino-pyridin-2-amine in step (a). MS (ESI): 348.2 ([M+H]⁺).

### Intermediate B5

### Ethyl 7-bromo-9-(morpholinomethyl)-4-oxo-pyrido[1,2-a]pyrimidine-3-carboxylate

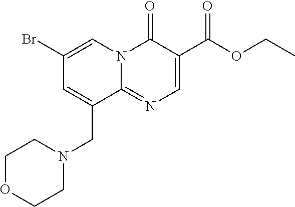 [SMILES: CCOC(=O)c1cnc2c(CN3CCOCC3)cc(Br)cn2c1=O]

The titled compound was synthesized according to the following scheme:

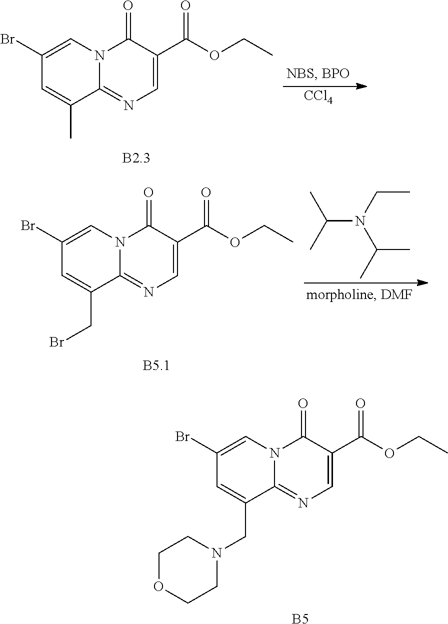

### Step (a) Preparation of ethyl 7-bromo-9-(bromomethyl)-4-oxo-pyrido[1,2-a]pyrimidine-3-carboxylate (Compound B5.1)

A mixture of ethyl 7-bromo-9-methyl-4-oxo-4H-pyrido[1,2-a]pyrimidine-3-carboxylate (1 g, 3.21 mmol, compound B2.3), NBS (686 mg, 3.86 mmol), and BPO (156 mg, 643 μmol) in CCl₄ (15 ml) was refluxed for 2 h. Afterwards, the mixture was cooled back to r.t., diluted with DCM (100 mL), and washed with aq. Na₂S₂O₃ solution. The separated organic layer was then dried over anhy. Na₂SO₄, filtered, and concentrated in vacuo to give a crude product of ethyl 7-bromo-9-(bromomethyl)-4-oxo-pyrido[1,2-a]pyrimidine-3-carboxylate. It was used directly in the next step without further purification. MS: 391.0 (Br⁷⁹[M+H]⁺), 393.1 (Br⁸¹ [M+H]⁺).

### Step (b) Preparation of Ethyl 7-bromo-9-(morpholinomethyl)-4-oxo-pyrido[1,2-a]pyrimidine-3-carboxylate (Intermediate B5)

A mixture of ethyl 7-bromo-9-(bromomethyl)-4-oxo-4H-pyrido[1,2-a]pyrimidine-3-carboxylate (1.25 g, 3.2 mmol), morpholine (558 mg, 558 μl, 6.41 mmol) and N-ethyl-N-isopropylpropan-2-amine (828 mg, 1.12 ml, 6.41 mmol) in DMF (5 mL) was stirred at room temperature for 1 h. Afterwards, the mixture was diluted with EtOAc (50 mL), and washed with brine. The separated organic layer was dried over anhy. Na₂SO₄, filtered, and concentrated in vacuo to give a crude product, which was purified by silica gel flash chromatography (petroleum ether:EtOAc=1:1) to give ethyl 7-bromo-9-(morpholinomethyl)-4-oxo-pyrido[1,2-a]pyrimidine-3-carboxylate (0.376 g, 29.6% yield). MS: 396.1 (Br⁷⁹[M+H]⁺), 398.0 (Br⁸¹ [M+H]⁺).

### Intermediate B6

### Ethyl 7-bromo-9-[[2-methoxyethyl(methyl)amino]methyl]-4-oxo-pyrido[1,2-a]pyrimidine-3-carboxylate

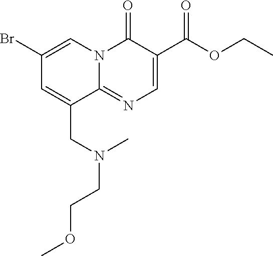 [SMILES: CCOC(=O)c1cnc2c(CN(C)CCOC)cc(Br)cn2c1=O]

In analogy to the synthesis of ethyl 7-bromo-9-(morpholinomethyl)-4-oxo-pyrido[1,2-a]pyrimidine-3-carboxylate (Intermediate B5), the title compound was prepared by replacing morpholine with 2-methoxy-N-methyl-ethanamine in step (b). MS: 398.1 (Br⁷⁹[M+H]⁺), 400.0 (Br⁸¹ [M+H]⁺).

### Intermediate B7

### Ethyl 7-bromo-4-oxo-9-(pyrrolidin-1-ylmethyl)pyrido pyrimidine-3-carboxylate

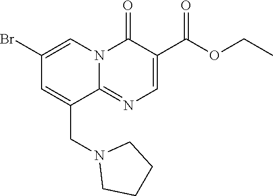 [SMILES: CCOC(=O)c1cnc2c(CN3CCCC3)cc(Br)cn2c1=O]

In analogy to the synthesis of ethyl 7-bromo-9-(morpholinomethyl)-4-oxo-pyrido[1,2-a]pyrimidine-3-carboxylate (Intermediate B5), the title compound was prepared by replacing morpholine with pyrrolidine in step (b). MS: 380.0 (Br⁷⁹[M+H]⁺), 382.1 (Be⁸¹ [M+H]⁺).

### Intermediate B8

### Ethyl 9-((dimethylamino)methyl)-4-oxo-7-(4,4,5,5-tetramethyl-1,3,2-dioxaborolan-2-yl)-411-pyrido[1,2-a]pyrimidine-3-carboxylate

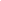

The titled compound was synthesized according to the following scheme:

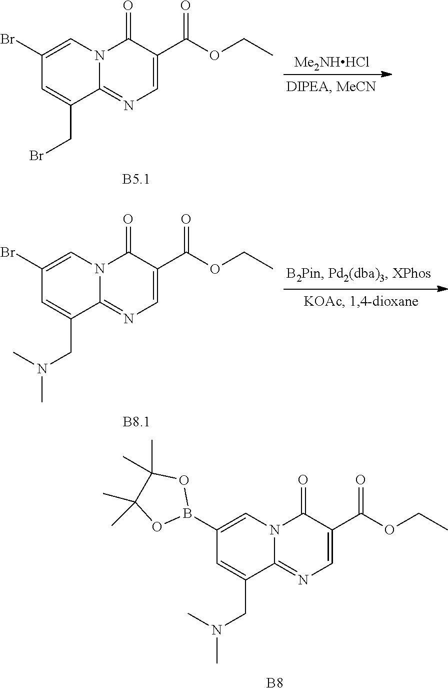

### Step (a) Preparation of ethyl 7-bromo-9-((dimethylamino)methyl)-4-oxo-4H-pyrido[1,2-a]pyrimidine-3-carboxylate (Compound B8.1)

In analogy to the synthesis of ethyl 7-bromo-9-(morpholinomethyl)-4-oxo-pyrido[1,2-a]pyrimidine-3-carboxylate (Intermediate B5), the title compound was prepared by replacing morpholine with dimethylamine in step (b). MS (ESI): 355.9 ([{⁸¹Br} M+H]⁺), 377.9 ([{⁸¹Br} M+Na]⁺).

### Step (d) Preparation of Ethyl 9-((dimethylamino)methyl)-4-oxo-7-(4,4,5,5-tetramethyl-1,3,2-dioxaborolan-2-yl)-4H-pyrido[1,2-a]pyrimidine-3-carboxylate (Intermediate B8)

A solution of ethyl 7-bromo-9-((dimethylamino)methyl)-4-oxo-4H-pyrido[1,2-a]pyrimidine-3-carboxylate (900 mg, 2.5 mmol), B₂Pin₂ (1.3 g, 5 mmol), and AcOK (0.48 mL, 7.6 mmol) in 1,4-dioxane (20 mL) was degassed with N₂ for five times before tris(dibenzylideneacetone)dipalladium (0) (116.3 mg, 0.13 mmol) and XPhos (121.1 mg, 0.25 mmol) were added to the mixture under N₂. The reaction solution was degassed again with N₂ for five times, and stirred at 100° C. for 12 h. The mixture was cooled back to r.t. and concentrated in vacuo to give a residue, which was diluted with DCM (500 mL) and filtered. The filtrate was concentrated in vacuo to give a crude product, which was purified by Prep-HPLC (0.5% TFA) to give ethyl 9-((dimethylamino)methyl)-4-oxo-7-(4,4,5,5-tetramethyl-1,3,2-dioxaborolan-2-yl)-4H-pyrido[1,2-a]pyrimidine-3-carboxylate (1 g, 82.3% yield) as a brown solid. MS (ESI): 320.1 ([M+H]⁺). ¹H NMR (MeOH-d4 400 MHz): δ 9.494˜9.497 (m, 1H), 9.05-79.066 (m, 1H), 8.446 (s, 1H), 4.720-4.740 (m, 2H), 4.381-4.434 (m, 2H), 3.06-63.079 (m, 6H), 1.264-1.272 (m, 15H).

### Intermediate B9

### Ethyl 7-bromo-9-[(5-methyl-2,3,3a,4,6,6a-hexahydropyrrolo[3,4-b]pyrrol-1-yl)methyl]-4-oxo-pyrido[1,2-a]pyrimidine-3-carboxylate

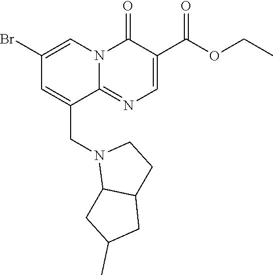 [SMILES: CCOC(=O)c1cnc2c(CN3CCC4CC(C)CC43)cc(Br)cn2c1=O]

In analogy to the synthesis of ethyl 7-bromo-9-(morpholinomethyl)-4-oxo-pyrido[1,2-a]pyrimidine-3-carboxylate (Intermediate B5), the title compound was prepared by replacing morpholine with 5-methyl-2,3,3a,4,6,6a-hexahydro-1H-pyrrolo[2,3-c]pyrrole in step (b). MS: 435.2 (Br⁷⁹ [M+H]⁺), 437.1 (Br⁸¹ [M+H]⁺).

### Intermediate C₁

### 1-(3-Fluoropyrrolidin-3-yl)-N,N-dimethylmethanamine Hydrochloride

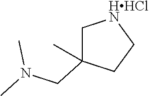

The titled compound was synthesized according to the following scheme:

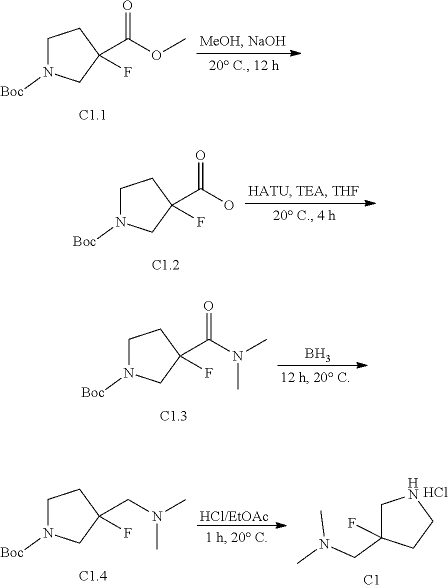

### Step (a) Preparation of 1-(tert-butoxycarbonyl)-3-fluoropyrrolidine-3-carboxylic Acid (Compound C1.2)

To a solution of 1-tert-butyl 3-methyl 3-fluoropyrrolidine-1,3-dicarboxylate (3.5 g, 14.1 mmol) in MeOH (10 mL) and water (2 mL) was added sodium hydroxide (1.7 g, 42.4 mmol), and the resulting reaction mixture was stirred at 20° C. for 12 h. The mixture was diluted with DCM (100 mL), adjusted to pH=3 with aq. HCl solution (2 M), and then extracted with DCM (100 mL). The organic layer was dried over anhy. Na₂SO₄, filtered, and concentrated in vacuo to give a crude product of 1-(tert-butoxycarbonyl)-3-fluoropyrrolidine-3-carboxylic acid (3.2 g, 96.9% yield) as a white solid. It was used directly in next step without further purification.

### Step (b) Preparation of Tert-Butyl 3-(dimethylcarbamoyl)-3-fluoropyrrolidine-1-carboxylate (Compound C1.3)

A mixture solution of 1-(tert-butoxycarbonyl)-3-fluoropyrrolidine-3-carboxylic acid (2.9 g, 12.4 mmol), HATU (6.1 g, 16.1 mmol), dimethylamine hydrochloride (5.1 g, 62.1 mmol), and DIPEA (6.5 mL, 37.3 mmol) in THF (10 mL) was stirred at 20° C. for 4 h. After LC-MS showed the starting material was consumed, the mixture was diluted with DCM (100 mL), poured into water (50 mL), and extracted with DCM (100 mL). The organic layer was washed with brine (50 mL), dried over anhy. Na₂SO₄, filtered, and concentrated in vacuo to give a crude product of tert-butyl 3-(dimethylcarbamoyl)-3-fluoropyrrolidine-1-carboxylate (1.9 g) as colorless oil. MS (ESI): 205.1 ([M+H-t-Bu]⁺). It was used directly in the next step without further purification.

### Step (c) Preparation of Tert-Butyl 3-((dimethylamino)methyl)-3-fluoropyrrolidine-1-carboxylate (Compound C1.4)

To a solution of tert-butyl 3-(dimethylcarbamoyl)-3-fluoropyrrolidine-1-carboxylate (1.8 g, 6.9 mmol) in THF (8 mL) was added borane-methyl sulfide complex (8 mL, 80 mmol) at 0° C., and the resulting reaction mixture was allowed to warm up to 20° C. and stirred for 12 h. The mixture was then poured into cold MeOH (100 mL) and stirred for additional 1 h before it was concentrated in vacuo to give a crude product of tert-butyl 3-((dimethylamino)methyl)-3-fluoropyrrolidine-1-carboxylate (1.2 g) as colorless oil. MS (ESI): 247.1 ([M+H]⁺), 191.0 ([M+H-t-Bu]⁺). It was used directly in the next step without further purification.

### Step (d) Preparation of 1-(3-fluoropyrrolidin-3-yl)-N,N-dimethylmethanamine hydrochloride (Compound C1)

A solution of tert-butyl 3-((dimethylamino)methyl)-3-fluoropyrrolidine-1-carboxylate (1.2 g, 4.8 mmol) in HCl/EtOAc (10 mL, 40 mmol, 4 M) was stirred at 20° C. for 2 h. After LC-MS showed the starting material was consumed, the mixture solution was concentrated in vacuo to give a crude product, which was recrystallized in MeOH/EtOAc (v:v=1:10, 10 mL) to give 1-(3-fluoropyrrolidin-3-yl)-N,N-dimethylmethanamine hydrochloride (910 mg, 85.2% yield) as a white solid. MS (ESI): 147.1 ([M+H]⁺).

### Intermediate C2

### 2-Methoxy-N-methyl-N-(pyrrolidin-3-ylmethyl)ethanamine Dihydrochloride

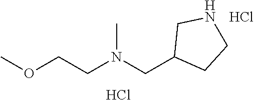

The titled compound was synthesized according to the following scheme:

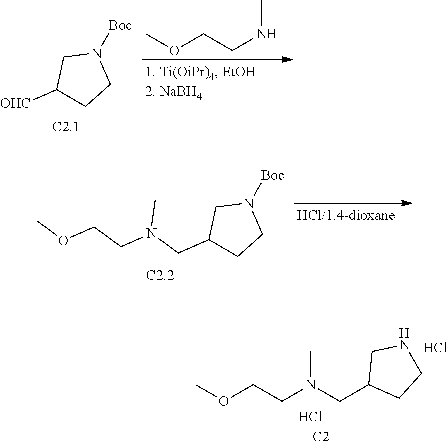

### Step (a) Preparation of Tert-Butyl 3-(((2-methoxyethyl)(methyl)amino)methyl)pyrrolidine-1-carboxylate (Compound C2.2)

To a solution of tert-butyl 3-formylpyrrolidine-1-carboxylate (2 g, 22.5 mmol) and 2-methoxy-N-methylethanamine (2 g, 10.1 mmol) in EtOH (40 mL), was added Ti(OiPr)₄ (4 g, 14.1 mmol) under N₂. The mixture was stirred at 15° C. for 12 h before NaBH₄ (1.6 g, 42.3 mmol) was added to the mixture and stirred at 15° C. for additional 12 h. The reaction mixture was then poured into water (250 mL) and filtered. The filtrate was extracted by EtOAc (100 mL) three times. Combined organics were washed with brine (100 mL) three times, dried over anhy. Na₂SO₄, filtered, and concentrated in vacuo to give a crude product, which was purified by silica gel flash chromatography (DCM:MeOH=20:1-10:1) to give tert-butyl 3-(((2-methoxyethyl) (methyl) amino) methyl)pyrrolidine-1-carboxylate (2.5 g, 92.6% yield) as light yellow oil. ¹H NMR (400 MHz, DMSO-d6) δ ppm 3.373˜3.402 (t, 2H), 3.342 (s, 2H), 3.277˜3.315 (m, 1H), 3.268 (s, 3H), 3.228 (s, 1H), 2.499 (s, 1H), 2.458-2.483 (s, 1H), 2.261˜2.276 (m, 2H), 2.177 (s, 3H), 1.840˜1.867 (m, 1H), 1.476˜1.496 (m, 1H), 1.240 (s, 9H).

### Step (b) Preparation of 2-methoxy-N-methyl-N-(pyrrolidin-3-ylmethyl) ethanamine Dihydrochloride (Intermediate C2)

A solution of tert-butyl 3-(((2-methoxyethyl)(methyl)amino)methyl)pyrrolidine-1-carboxylate (2.5 g, 9.178 mmol) in HCl/dioxane (25 mL) was stirred at 15° C. for 1 h. Afterwards, the reaction mixture was concentrated in vacuo to give a crude product of 2-methoxy-N-methyl-N-(pyrrolidin-3-ylmethyl) ethanamine dihydrochloride (1.5 g) as thick yellow oil. MS (ESI): 173.1 ([M+H]⁺). It was used directly in the next step without further purification.

### Intermediate C3

### 2,2-Difluoro-N-methyl-N-(pyrrolidin-3-ylmethyl)ethanamine Dihydrochloride

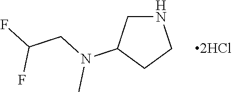

The titled compound was synthesized according to the following scheme:

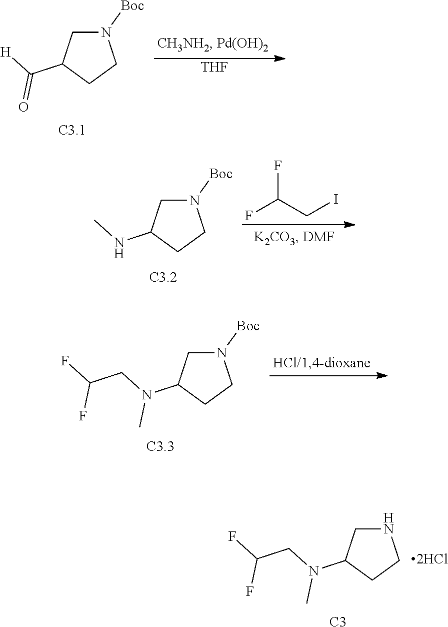

### Step (a) Preparation of tert-butyl 3-((methylamino)methyl)pyrrolidine-1-carboxylate (compound C3.2)

To a solution of tert-butyl 3-formylpyrrolidine-1-carboxylate (4 g, 20.08 mmol) in CH₃NH₂/THF (25 mL) was added Pd(OH)₂ (1.4 g, 10.04 mmol), and the resulting mixture was stirred at 25° C. for 5 h under H₂ atmosphere (50 psi). After TLC (DCM:MeOH=10:1) showed the starting material was consumed, the reaction mixture was filtered and the filtrate was concentrated in vacuo to give a crude product of tert-butyl 3-((methylamino)methyl)pyrrolidine-1-carboxylate (3.8 g, 88.4% yield) as yellow oil. It was used directly in the next step without further purification.

### Step (b) Preparation of tert-butyl 3-4(2,2-difluoroethyl)(methyl)amino)methyl)pyrrolidine-1-carboxylate (Compound C3.3)

To a solution of 1,1-difluoro-2-iodoethane (2.7 g, 14.02 mmol) and tert-butyl 3-((methylamino)methyl)pyrrolidine-1-carboxylate (2 g, 9.35 mmol) in DMF (5 mL), was added K₂CO₃ (2.6 g, 18.70 mmol), and the resulting reaction mixture was stirred at 60° C. for 12 h. After the reaction was cooled back to r.t., the mixture solution was extracted by EtOAc (100 mL) three times. Combined organics were washed with brine (100 mL) three times, dried over anhy. Na₂SO₄, filtered, and concentrated in vacuo to give a crude product, which was purified by silica gel flash chromatography (petroleum ether:EtOAc=10:1˜3:1) to give tert-butyl 3-(((2,2-difluoroethyl) (methyl)amino)methyl)pyrrolidine-1-carboxylate (2.3 g, 53.5% yield) as light yellow oil.

### Step (c) Preparation of 2,2-difluoro-N-methyl-N-(pyrrolidin-3-ylmethyl)ethanamine Dihydro Chloride (Intermediate C₃)

A mixture of tert-butyl 3-(((2,2-difluoroethyl)(methyl)amino)methyl)pyrrolidine-1-carboxylate (2.3 g, 8.264 mmol) in HCl/dioxane solution (25 mL, 4 M) was stirred at 15° C. for 1 h. Afterwards, the reaction mixture was concentrated in vacuo to give a crude product of 2,2-difluoro-N-methyl-N-(pyrrolidin-3-ylmethyl)ethanamine dihydrochloride (1.5 g) as thick yellow oil. It was used directly in the next step without further purification. ¹HNMR (400 MHz, DMSO-d6) δ: 5.613˜5.880 (t, 1H), 3.045˜3.078 (m, 2H), 2.860˜2.869 (m, 2H), 2.734˜2.745 (s, 3H), 2.692 (s, 1H), 2.298 (s, 3H), 2.136 (s, 1H), 1.605˜1.630 (m, 1H), 1.046˜1.100 (m, 1H).

### Intermediate C₄

### 1-(4,4-Difluoropyrrolidin-3-yl)-N,N-dimethylmethanamine

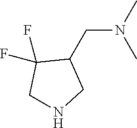

The titled compound was synthesized according to the following scheme:

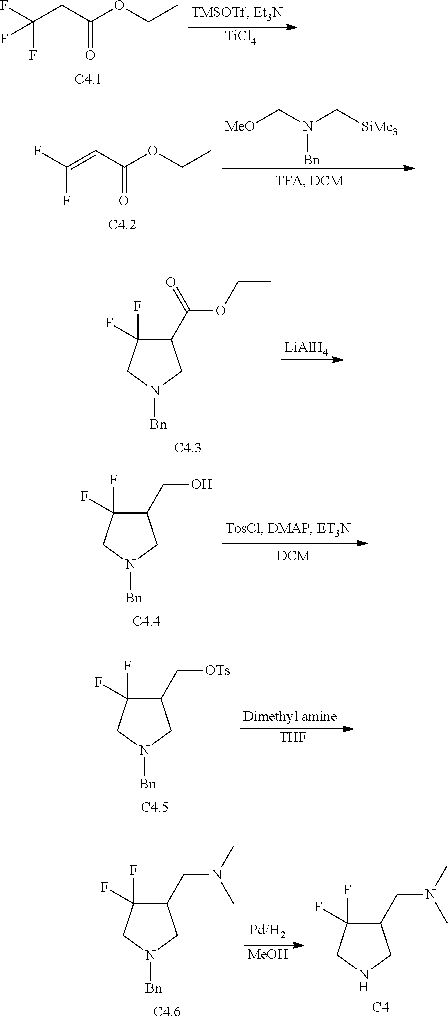

### Step (a) Preparation of Ethyl 3,3-difluoroacrylate (Compound C4.2)

To a solution of ethyl 3,3,3-trifluoropropanoate (10 g, 64.1 mmol) in CHCl₃ (100 mL) was added Et₃N (7.8 g, 76.9 mmol), and TMSOTf (17.4 g, 76.9 mmol) dropwise. After the addition, the mixture was stirred at r.t. for 1.5 h. The reaction was then cooled in an ice bath, and a solution of TiCl₄ in DCM was added (9.61 mL, 1 M). The resulting reaction mixture was allowed to warm up to r.t. and stirred for 1 h before water (100 mL) was added to quench the reaction under an ice bath. Separated organic layer was then dried over anhy. MgSO₄, filtered, and concentrated in vacuo to give a crude product of ethyl 3,3-difluoroacrylate. It was used directly in the next step without further purification.

### Step (b) Preparation of Ethyl 1-benzyl-4,4-difluoropyrrolidine-3-carboxylate (Compound C4.3)

To a solution of N-(methoxymethyl)-1-phenyl-N-(trimethylsilylmethyl)methanamine (5.0 g, 36.8 mmol) in CHCl₃ (20 mL) was added ethyl 3,3-difluoroacrylate (1.3 g, 55.1 mmol) and TFA (0.5 mL) in an ice bath. The resulting reaction mixture was stirred for 1 h in an ice bath before it was allowed to warm up to r.t. and stirred for additional 2 h. Saturated aq. NaHCO₃ solution was added to quench the reaction and the mixture was poured into water (50 mL) and extracted with EtOAc (50 mL). The organic layer was washed with aq. CaCl₂) solution (100 mL, 1N) two times, brine (100 mL), dried over anhy. Na₂SO₄, filtered, and concentrated in vacuo. The crude product was purified by silica gel flash chromatography (petroleum ether:EtOAc=40:1-20:1) to give ethyl 1-benzyl-4,4-difluoropyrrolidine-3-carboxylate (10.9 g, 74.7% yield) as an oil. MS (ESI): 270.1 ([M+H]⁺).

### Step (c) Preparation of (1-benzyl-4,4-difluoropyrrolidin-3-yl)methanol (Compound C4.4)

A solution of ethyl 1-benzyl-4,4-difluoropyrrolidine-3-carboxylate (4 g, 14.9 mmol) and LiAlH₄ (5.6 g, 148.6 mmol) in THF (20 mL) was stirred at 25° C. for 2 h. Afterwards, the reaction was quenched with aq. NaOH solution and filtered. The filtrate was poured into water and extracted with EtOAc. The separated organic layer was washed with aq. CaCl₂) solution (50 mL, 1N) two times, brine (50 mL), dried over anhy. Na₂SO₄, filtered, and concentrated in vacuo to give a crude product of (1-benzyl-4,4-difluoropyrrolidin-3-yl)methanol (3.1 g) as yellow oil. MS (ESI): 228.1 ([M+H]⁺).

### Step (d) Preparation of (1-benzyl-4,4-difluoropyrrolidin-3-yl)methyl 4-methylbenzenesulfonate (Compound C4.5)

To a solution of (1-benzyl-4,4-difluoropyrrolidin-3-yl)methanol (1 g, 4.4 mmol) in DCM (10 mL) was added TosCl (1.3 g, 6.6 mmol), Et₃N (1.3 g, 13.2 mmol), and DMAP (0.16 g, 1.32 mmol) at 0° C. The resulting reaction mixture was stirred at 0° C. for 2 h before concentrated in vacuo. The crude product was purified by prep-HPLC (0.1% TFA as additive) to give (1-benzyl-4,4-difluoropyrrolidin-3-yl)methyl 4-methylbenzenesulfonate (1.2 g, 70.6% yield) as colorless oil. MS (ESI): 382.0 (M+H)⁺.

### Step (d) Preparation of 1-(1-benzyl-4,4-difluoropyrrolidin-3-yl)-N,N-dimethylmethanamine (Compound C4.6)

To a solution of (1-benzyl-4,4-difluoropyrrolidin-3-yl)methyl 4-methylbenzenesulfonate (1.2 g, 3.15 mmol) in THF (10 mL) was added dimethylamine (30 mL, 2M in THF), and the resulting mixture was stirred at 60° C. for 2 h. The mixture was then concentrated in vacuo to give a crude product, which was purified by prep-HPLC (0.5% TFA in water) to give 1-(1-benzyl-4,4-difluoropyrrolidin-3-yl)-N,N-dimethylmethanamine (0.65 g, 81.2% yield) as colorless oil. MS (ESI): 255.1 (M+H)⁺.

### Step (e) Preparation of 1-(4,4-difluoropyrrolidin-3-yl)-N,N-dimethylmethanamine (Intermediate C4)

After degassed and purged with H₂ several times, a solution of 1-(1-benzyl-4,4-difluoropyrrolidin-3-yl)-N,N-dimethylmethanamine (0.65 g, 2.56 mmol) and Pd(OH)₂ (0.36 g, 0.26 mmol) in MeOH (15 mL) was stirred at 60° C. for 12 h under H₂ (50 psi). Afterwards, the reaction was cooled back to r.t., and filtered through Celite. The filter cake was washed with MeOH (30 mL) four times, and combined organics was concentrated in vacuo to give a crude product of 1-(4,4-difluoropyrrolidin-3-yl)-N,N-dimethylmethanamine (0.79 g) as colorless oil. MS (ESI): 165.1 (M+H)⁺. It was used directly in the next step without further purification.

### Example 1.01

### 7-[4-(Dimethylamino)-5,6-difluoro-8-(methylamino)-9H-pyrido[2,3-b]indol-3-yl]-4-oxo-pyrido[1,2-a]pyrimidine-3-carboxylic Acid

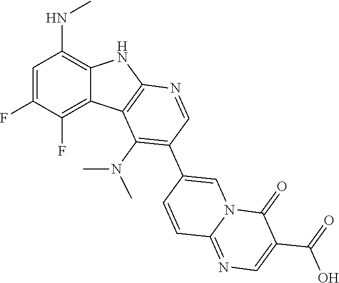 [SMILES: CNc1cc(F)c(F)c2c1[nH]c1ncc(-c3ccc4ncc(C(=O)O)c(=O)n4c3)c(N(C)C)c12]

### Step (a) Preparation of tert-butyl N-[3-chloro-4-(dimethylamino)-5,6-difluoro-9H-pyrido[2,3-b]indol-8-yl]-N-methyl-carbamate

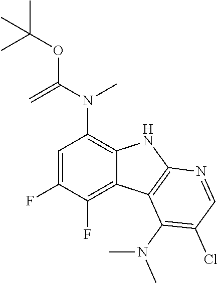 [SMILES: C=C(OC(C)(C)C)N(C)c1cc(F)c(F)c2c1[nH]c1ncc(Cl)c(N(C)C)c12]

To the solution of tert-butyl N-(3,4-dichloro-5,6-difluoro-9H-pyrido[2,3-b]indol-8-yl)-N-methyl-carbamate (Intermediate A3, 400 mg, 1 mmol, as the “CORE” in Table 1) in DMSO (5 mL) were added N-methylmethanamine hydrochloride (105 mg, 1.3 mmol, as the “AMINE” in Table 1) and DIPEA (771 mg, 6 mmol), and the resulting mixture was stirred at 120° C. for 12 h. The reaction mixture was cooled back to room temperature, filtered through thin Celite pad, and concentrated in vacuo. The residue was purified by prep-HPLC to give tert-butyl N-[3-chloro-4-(dimethylamino)-5,6-difluoro-9H-pyrido[2,3-b]indol-8-yl]-N-methyl-carbamate (241 mg, 59% yield) as a white solid. MS (ESI): 411.1.

### Step (b) Preparation of Ethyl 7-[8-[tert-butoxycarbonyl(methyl)amino]-4-(dimethylamino)-5,6-difluoro-9H-pyrido[2,3-b]indol-3-yl]-4-oxo-pyrido[1,2-a]pyrimidine-3-carboxylate

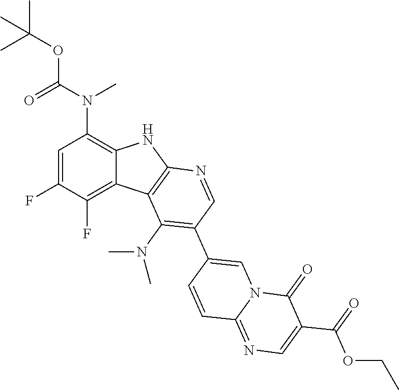 [SMILES: CCOC(=O)c1cnc2ccc(-c3cnc4[nH]c5c(N(C)C(=O)OC(C)(C)C)cc(F)c(F)c5c4c3N(C)C)cn2c1=O]

A solution of tert-butyl N-[3-chloro-4-(dimethylamino)-5,6-difluoro-9H-pyrido[2,3-b]indol-8-yl]-N-methyl-carbamate (42 mg, 0.102 mmol), (3-ethoxycarbonyl-4-oxo-pyrido[1,2-a]pyrimidin-7-yl)boronic acid (32.1 mg, 0.123 mmol, as the “BORONIC REAGENT” in Table 1), Xphos Pd G2 (8 mg, 0.01 mmol, CAS: 1310584-14-5), as the “CATALYST” in Table 1, and potassium phosphate tribasic (43.4 mg, 0.20 mmol) in THF (3 mL) and water (1 mL) was stirred at 60° C. for 12 h under N₂ atmosphere. Afterwards, the mixture was cooled back to room temperature, poured into water (50 mL), and extracted with EtOAc (50 mL) three times. The combined organics were dried over anhy. Na₂SO₄, filtered, and concentrated in vacuo to give a crude product of ethyl 7-[8-[tert-butoxycarbonyl(methyl)amino]-4-(dimethylamino)-5,6-difluoro-9H-pyrido[2,3-b]indol-3-yl]-4-oxo-pyrido[1,2-a]pyrimidine-3-carboxylate (60 mg). MS (ESI): 593.2 ([M+H]⁺). It was used directly in the next step without further purification.

### Step (c) Preparation of 7-[8-[tert-butoxycarbonyl(methyl)amino]-4-(dimethylamino)-5,6-difluoro-9H-pyrido[2,3-b]indol-3-yl]-4-oxo-pyrido[1,2-a]pyrimidine-3-carboxylic Acid

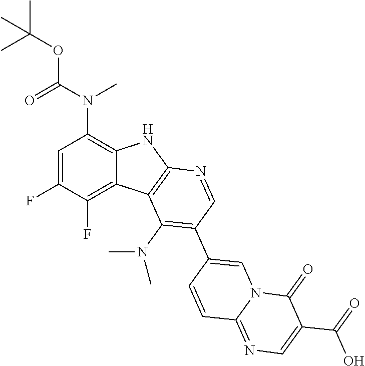 [SMILES: CN(C)c1c(-c2ccc3ncc(C(=O)O)c(=O)n3c2)cnc2[nH]c3c(N(C)C(=O)OC(C)(C)C)cc(F)c(F)c3c12]

To a stirred solution of ethyl 7-(8-((tert-butoxycarbonyl)(methyl)amino)-4-(dimethylamino)-5,6-difluoro-9H-pyrido[2,3-b]indol-3-yl)-4-oxo-4H-pyrido[1,2-a]pyrimidine-3-carboxylate (0.06 g, 101 μmol) in THF (3 mL) was added sodium hydroxide (40.5 mg, 1.01 mmol) at room temperature, and the resulting mixture was stirred at room temperature overnight. The mixture was then concentrated in vacuo to give a crude product of 7-[8-[tert-butoxycarbonyl(methyl)amino]-4-(dimethylamino)-5,6-difluoro-9H-pyrido[2,3-b]indol-3-yl]-4-oxo-pyrido[1,2-a]pyrimidine-3-carboxylic acid. MS (ESI): 565.2 ([M+H]⁺). It was used directly in the next step without further purification.

### Step (d) Preparation of 7-[4-(dimethylamino)-5,6-difluoro-8-(methylamino)-9H-pyrido[2,3-b]indol-3-yl]-4-oxo-pyrido[1,2-a]pyrimidine-3-carboxylic Acid

To a stirred solution of 7-(8-((tert-butoxycarbonyl)(methyl)amino)-4-(dimethylamino)-5,6-difluoro-9H-pyrido[2,3-b]indol-3-yl)-4-oxo-4H-pyrido[1,2-a]pyrimidine-3-carboxylic acid (0.057 g, 101 μmol) in DCM (3 mL) was added TFA (3 g, 2 mL, 26.3 mmol) at room temperature. The resulting reaction mixture was stirred at room temperature for 2 h before concentrated in vacuo. The residue was purified by prep-HPLC to give 7-[4-(dimethylamino)-5,6-difluoro-8-(methylamino)-9H-pyrido[2,3-b]indol-3-yl]-4-oxo-pyrido[1,2-a]pyrimidine-3-carboxylic acid (2.6 mg, 5.5% yield) as a yellow solid. ¹H NMR (MeOH-d4, 400 MHz) δ 9.1-9.2 (m, 1H), 9.0-9.1 (m, 1H), 8.12 (s, 2H), 7.9-8.0 (m, 1H), 6.5-6.6 (m, 1H), 2.9-2.9 (m, 3H), 2.8-2.9 (m, 6H). MS (ESI): 465.3 ([M+H]⁺).

The following examples were prepared in analogy to Example 1.01, by replacing tert-butyl N-(3,4-dichloro-5,6-difluoro-9H-pyrido[2,3-b]indol-8-yl)-N-methyl-carbamate (Intermediate A3) as the “CORE” in step (a), N-methylmethanamine hydrochloride as the “AMINE” in step (a), (3-ethoxycarbonyl-4-oxo-pyrido[1,2-a]pyrimidin-7-yl)boronic acid as the “BORONIC REAGENT” in step (b), and Xphos Pd G2 (CAS: 1310584-14-5) as the “CATALYST” in step (b) by the reagents indicated in Table 1.

TABLE 1
Compound synthesis and characterization

| | | CORE, AMINE, | |
|---|---|---|---|
| | | BORONIC REAGENT, | |
| | Compound Name and | CATALYST and | |
| No. | Structure | SOLVENT | ¹H NMR and MS (ESI) |
| 1.02 | 7-[4-(Dimethylamino)-5,6- difluoro-8-(methylamino)- 9H-pyrido[2,3-b]indol-3-yl]- 9-methyl-4-oxo-pyrido[1,2- a]pyrimidine-3-carboxylic acid 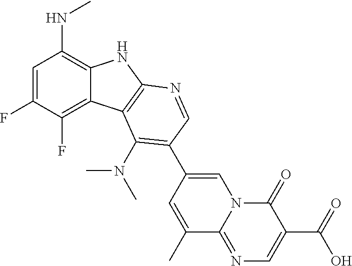 [SMILES: CNc1cc(F)c(F)c2c1[nH]c1ncc(-c3cc(C)c4ncc(C(=O)O)c(=O)n4c3)c(N(C)C)c12] | CORE: Intermediate A3 AMINE: N- methylmethanamine hydrochloride BORONIC REAGENT: B2 CATALYST: Xphos Pd G2 (CAS: 1310584-14-5) SOLVENT: THF/H₂O | ¹H NMR (DMSO-d₆, 400 MHz) δ 8.9-9.0 (m, 1H), 8.7-8.8 (m, 1H), 8.1-8.2 (m, 1H), 7.9-8.0 (m, 1H), 6.50 (br dd, 1H, J = 5.3, 13.4 Hz), 2.7-2.9 (m, 6H), 2.67 (br s, 3H), 2.56 (br d, 3H, J = 7.0 Hz) MS: 479.2 ([M + H]⁺) |
| 1.03 | 7-[4-(Dimethylamino)-5,6- difluoro-8-(methylamino)- 9H-pyrido[2,3-b]indol-3-yl]- 9-methoxy-4-oxo- pyrido[1,2-a]pyrimidine-3- carboxylic acid 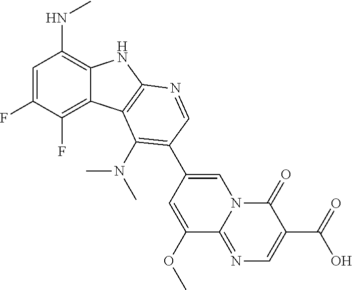 [SMILES: CNc1cc(F)c(F)c2c1[nH]c1ncc(-c3cc(OC)c4ncc(C(=O)O)c(=O)n4c3)c(N(C)C)c12] | CORE: Intermediate A3 AMINE: N- methylmethanamine hydrochloride BORONIC REAGENT: B3 CATALYST: Xphos Pd G2 (CAS: 1310584-14-5) SOLVENT: THF/H₂O | ¹H NMR (DMSO-d₆, 400 MHz) δ 8.8-8.8 (m, 1H), 8.53 (br d, 1H, J = 3.1 Hz), 8.2-8.2 (m, 1H), 1.5-1.5 (m, 1H), 6.5-6.6 (m, 1H), 5.56 (br d, 1H, J = 4.4 Hz), 3.99 (s, 3H), 2.83 (br d, 3H, J = 4.6 Hz), 2.74 (d, 6H, J = 1.0 Hz) MS: 495.1 ([M + H]⁺) |
| 1.04 | 7-[4-(Dimethylamino)-6- fluoro-8-(methylamino)-9H- pyrido[2,3-b]indol-3-yl]-4- oxo-pyrido[1,2- a]pyrimidine-3-carboxylic acid 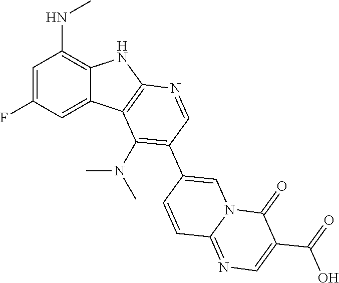 [SMILES: CNc1cc(F)cc2c1[nH]c1ncc(-c3ccc4ncc(C(=O)O)c(=O)n4c3)c(N(C)C)c12] | CORE: Intermediate A1 AMINE: N- methylmethanamine hydrochloride BORONIC REAGENT: B1 CATALYST: Xphos Pd G2(CAS: 1310584-14-5) SOLVENT: THF/H₂O | ¹H NMR (METHANOL-d₄, 400 MHz) δ 9.2-9.3 (m, 1H), 9.0-9.1 (m, 1H), 8.1- 8.2 (m, 2H), 7.9-8.0 (m, 2H), 6.9-7.0 (m, 1H), 6.4- 6.5 (m, 1H), 2.9-3.0 (m, 6H), 2.91 (s, 3H) MS: 447.4 ([M + H]⁺) |
| 1.05 | 7-[4-(Dimethylamino)-6- fliioro-8-(methylamino)-9H- pyrido[2,3-b]indol-3-yl]-9- methyl-4-oxo-pyrido[1,2- a]pyrimidine-3-carboxylic acid 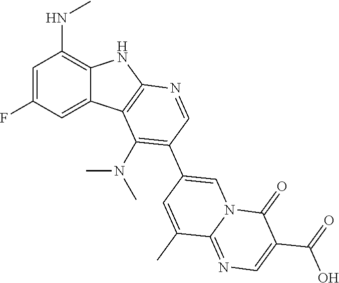 [SMILES: CNc1cc(F)cc2c1[nH]c1ncc(-c3cc(C)c4ncc(C(=O)O)c(=O)n4c3)c(N(C)C)c12] | CORE: Intermediate A1 AMINE: N- methylmethanamine hydrochloride BORONIC REAGENT: B2 CATALYST: Xphos Pd G2 (CAS: 1310584-14-5) SOLVENT: THF/H₂O | ¹H NMR (METHANOL-d₄, 400 MHz) δ 9.0-9.1 (m, 2H), 8.1-8.2 (m, 1H), 8.0- 8.1 (m, 1H), 6.9-7.0 (m, 1H), 6.4-6.5 (m, 1H), 2.9- 2.9 (m, 6H), 2.91 (s, 3H), 2.68 (s, 3H) MS: 461.4 ([M + H]⁺) |
| 1.06 | 7-[4-(Dimethylamino)-5,6- difluoro-8-(methylamino)- 9H-pyrido[2,3-b]indol-3-yl]- 9-morpholino-4-oxo- pyrido[1,2-a]pyrimidine-3- carboxylic acid 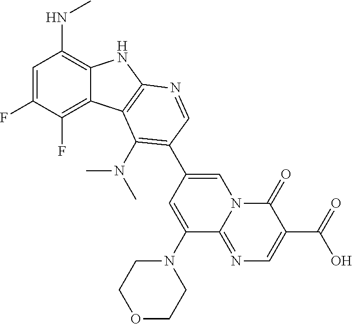 [SMILES: CNc1cc(F)c(F)c2c1[nH]c1ncc(-c3cc(N4CCOCC4)c4ncc(C(=O)O)c(=O)n4c3)c(N(C)C)c12] | CORE: Intermediate A3 AMINE: N- methylmethanamine hydrochloride BORONIC REAGENT: B4 CATALYST: Xphos Pd G2 (CAS: 1310584-14-5) SOLVENT: THF/H₂O | ¹H NMR (DMSO-d₆, 400 MHz) δ 11.7-11.9 (m, 1H), 8.9-9.0 (m, 1H), 8.7-8.8 (m, 1H), 8.2-8.3 (m, 1H), 7.4- 7.6 (m, 1H), 6.5-6.7 (m, 1H), 3.8-3.9 (m, 4H), 3.3- 3.5 (4H, m), 2.9-2.9 (m, 3H), 2.8-2.8 (m, 6H) MS: 550.1 ([M + H]⁺) |
| 1.07 | 7-[5,6-Difluoro-8- (methylamino)-4- pyrrolidin-1-yl-9H- pyrido[2,3-b]indol-3-yl]-9- morpholino-4-oxo- pyrido[1,2-a]pyrimidine-3- carboxylic acid 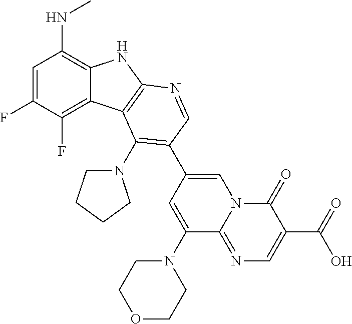 [SMILES: CNc1cc(F)c(F)c2c1[nH]c1ncc(-c3cc(N4CCOCC4)c4ncc(C(=O)O)c(=O)n4c3)c(N3CCCC3)c12] | CORE: Intermediate A3 AMINE: Pyrrolidine BORONIC REAGENT: B4 CATALYST: Xphos Pd G2 (CAS: 1310584-14-5) SOLVENT: THF/H₂O | ¹H NMR (DMSO-d₆, 400 MHz) δ 11.7-11.8 (m, 1H), 8.8-8.9 (m, 1H), 8.7-8.8 (m, 1H), 8.1-8.2 (m, 1H), 7.4- 7.5 (m, 1H), 7.0-7.2 (m, 1H), 6.4-6.6 (m, 1H), 3.7- 3.8 (m, 8H), 3.18 (br d, 8H, J = 5.3 Hz), 2.83 (s, 3H) MS: 576.2 ([M + H]⁺) |
| 1.08 | 7-[5,6-Difluoro-4-(5-methyl- 2,3,3a,4,6,6a- hexahydropyrrolo[2,3- c]pyrrol-1-yl)-8- (methylamino)-9H- pyrido[2,3-b]indol-3-yl]-4- oxo-pyrido[1,2- a]pyrimidine-3-carboxylic acid 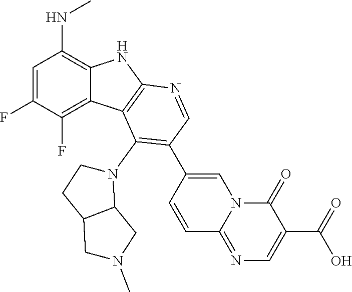 [SMILES: CNc1cc(F)c(F)c2c1[nH]c1ncc(-c3ccc4ncc(C(=O)O)c(=O)n4c3)c(N3CCC4CN(C)CC43)c12] | CORE: Intermediate A3 AMINE: 5-Methyl- 2,3,3a,4,6,6a-hexahydro- 1H-pyrrolo[3,4-b]pyrrole BORONIC REAGENT: Intermediate B1 CATALYST: Pd- Ad₂nBuP Biphenyl Precat (CAS: 1375477- 29-4) SOLVENT: THF/H₂O | ¹H NMR (400 MHz, DMSO-d6): ppm 11.80 (br. s., 1 H), 8.95 (br. s., 1 H), 8.76 (br. s., 1 H), 8.19 (d, J = 7.82 Hz, 1 H), 8.09 (s, 1 H), 7.74 (d, J = 8.07 Hz, 1 H), 6.51 (d, J = 13.08, 6.72 Hz, 1 H), 5.70 (br. s., 1 H), 4.39 (br. s., 1 H), 3.08 (br. s., 2 H), 2.68- 2.88 (m, 4 H), 2.24-2.37 (m, 2 H), 1.83-2.14 (m, 5 H), 1.56-1.74 (m, 2 H) MS (ESI):: 546.1 ([M + H]⁺). |
| 1.09 | 7-(5,6-Difluoro-4- (methyl(2,2,2- trifluoroethyl)amino)-8- (methylamino)-9H- pyrido[2,3-b]indol-3-yl)-4- oxo-4H-pyrido[1,2- a]pyrimidine-3-carboxylic acid 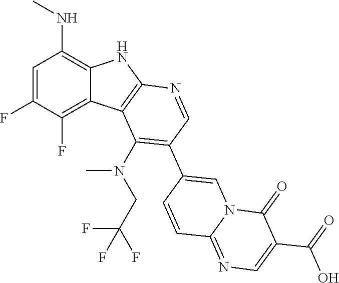 [SMILES: CNc1cc(F)c(F)c2c1[nH]c1ncc(-c3ccc4ncc(C(=O)O)c(=O)n4c3)c(N(C)CC(F)(F)F)c12] | CORE: Intermediate A3 AMINE: 2,2,2-Trifluoro- N-methylethanamine BORONIC REAGENT: Intermediate B1 CATALYST: Pd- Ad₂nBuP Biphenyl Precat (CAS: 1375477- 29-4) SOLVENT: THF/H₂O | ¹H NMR (400 MHz, DMSO): δ 11.97 (br s, 1 H), 9.14 (s, 1 H), 8.99 (s, 1 H), 8.41 (s, 1 H), 8.27~8.31 (m, 1 H), 8.03~8.05 (d, J = 8.909 Hz, 1 H), 6.62~ 6.66 (br dd, J = 13.427, 6.149 Hz, 1 H), 3.17 (s, 3 H), 3.10 (s, 1 H), 2.89~2.91 (m, 4 H). MS (ESI): 533.0 ([M + H]⁺) |
| 1.10 | 7-(5,6-Difluoro-8- (methylamino)-4- morpholino-9H-pyrido[2,3- b]indol-3-yl)-4-oxo-4H- pyrido[1,2-a]pyrimidine-3- carboxylic acid 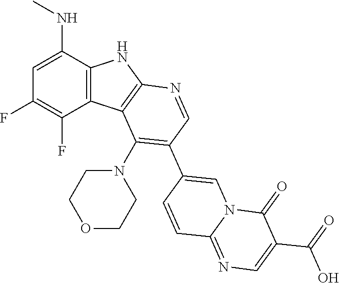 [SMILES: CNc1cc(F)c(F)c2c1[nH]c1ncc(-c3ccc4ncc(C(=O)O)c(=O)n4c3)c(N3CCOCC3)c12] | CORE: Intermediate A3 AMINE: Morpholine BORONIC REAGENT: Intermediate B1 CATALYST: Pd- Ad₂nBuP Biphenyl Precat (CAS: 1375477- 29-4) SOLVENT: THF/H₂O | ¹H NMR (400 MHz, DMSO-d6) δ ppm 9.09 (d, J = 1.63 Hz, 1 H), 8.93 (s, 1 H), 8.28 (dd, J = 8.97, 1.94 Hz, 1 H), 8.24 (s, 1 H), 7.96 (d, J = 9.03 Hz, 1 H), 6.62 (dd, J = 13.43, 6.15 Hz, 1 H), 5.78 (d, J = 3.76 Hz, 1 H), 3.63 (s, 4 H), 2.99-3.07 (m, 4 H), 2.53-2.58 (m, 3 H). MS (ESI): 507.0 ([M + H]⁺). |
| 1.11 | 7-(6-Fluoro-8- (methylamino)-4- morpholino-9H-pyrido[2,3- b]indol-3-yl)-4-oxo-4H- pyrido[1,2-a]pyrimidine-3- carboxylic acid 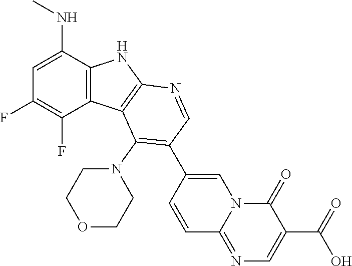 | CORE: Intermediate A1 AMINE: Morpholine BORONIC REAGENT: Intermediate B1 CATALYST: Pd- Ad₂nBuP Biphenyl Precat (CAS: 1375477- 29-4) SOLVENT: THF/H₂O | ¹H NMR (400 MHz, DMSO-d6) δ ppm 11.66 (s, 1 H), 9.09 (d, J = 1.63 Hz, 1 H), 8.96 (s, 1 H), 8.29 (dd, J = 8.91, 2.01 Hz, 1 H), 8.21 (s, 1 H), 7.98 (d, J = 8.91 Hz, 1 H), 7.10 (dd, J = 9.91, 2.13 Hz, 1 H), 6.47 (dd, J = 12.05, 2.01 Hz, 1 H), 5.98 (br d, J = 4.64 Hz, 1 H), 3.75 (s, 4 H), 3.06 (s, 4 H), 2.93 (d, J = 4.64 Hz, 3 H). MS: 465.1 ([M + H]⁺). |
| 1.12 | 7-(4-(Dimethylamino)-6,7- difluoro-8-(methylamino)- 9H-pyrido[2,3-b]indol-3-yl)- 4-oxo-4H-pyrido[1,2- a]pyrimidine-3-carboxylic 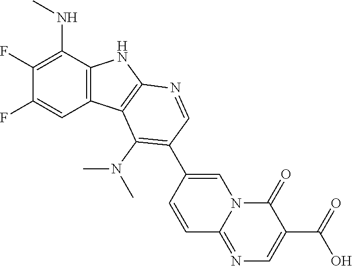 [SMILES: CNc1c(F)c(F)cc2c1[nH]c1ncc(-c3ccc4ncc(C(=O)O)c(=O)n4c3)c(N(C)C)c12] | CORE: Intermediate A4 AMINE: Dimethylamine hydrochloride BORONIC REAGENT: Intermediate B1 CATALYST: Pd- Ad₂nBuP Biphenyl Precat (CAS: 1375477- 29-4) SOLVENT: THF/H₂O | ¹H NMR (400 MHz, DMSO-d₆) δ ppm: 11.820 (s, 1H), 9.064~9.068 (d, J = 1.6, 1H), 8.971 (s, 1H), 8.221~8.248 (m, 2H), 7.982~8.004 (d, J = 8.8, 1H) 7.233~7.277 (m, 1H) 3.109 (s, 3H), 2.865 (s, 6H). MS: 465.1 ([M + H]⁺). |
| 1.13 | 7-(6,7-Difluoro-8- (methylamino)-4- morpholino-9H-pyrido[2,3- b]indol-3-yl)-4-oxo-4H- pyrido[1,2-a]pyrimidine-3- carboxylic acid 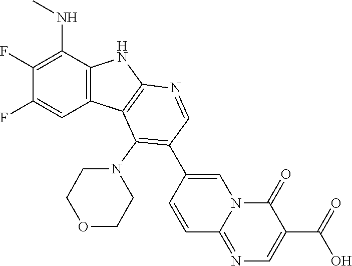 [SMILES: CNc1c(F)c(F)cc2c1[nH]c1ncc(-c3ccc4ncc(C(=O)O)c(=O)n4c3)c(N3CCOCC3)c12] | CORE: Intermediate A4 AMINE: Morpholine BORONIC REAGENT: Intermediate B1 CATALYST: Pd- Ad₂nBuP Biphenyl Precat (CAS: 1375477- 29-4) SOLVENT: THF/H₂O | ¹H NMR (400 MHz, DMSO-d₆) δ ppm 11.813 (s, 1H), 9.091~9.096 (d, J = 2.0 Hz, 1H), 8.986 (s, 1H), 8.299~8.326 (m, 1H), 8.215 (s, 1H), 7.989~8.011 (d, J = 8.8 Hz, 1H), 7.289~7.334 (m, 1H), 3.727~3.748 (t, 4H), 3.118~3.129 (d, J = 4.4 Hz, 3H), 3.033~3.042 (d, J = 3.6 Hz, 1H). MS: 507.1 ([M + H]⁺), 525.1 ([M + Na]⁺). |
| 1.14 | 7-(5,6-Difluoro-8- (methylamino)-4- morpholino-9H-pyrido[2,3- b]indol-3-yl)-9- ((dimethylamino)methyl)-4- oxo-4H-pyrido[1,2- a]pyrimidine-3-carboxylic acid 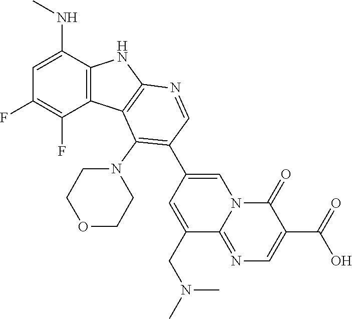 [SMILES: CNc1cc(F)c(F)c2c1[nH]c1ncc(-c3cc(CN(C)C)c4ncc(C(=O)O)c(=O)n4c3)c(N3CCOCC3)c12] | CORE: Intermediate A3 AMINE: Morpholine BORONIC REAGENT: Intermediate B8 CATALYST: Pd- Ad₂nBuP Biphenyl Precat (CAS: 1375477- 29-4) SOLVENT: THF/H₂O | ¹H NMR (400 MHz, MeOD): δ ppm 9.388~9.393 (m, 1 H), 9.210 (s, 1 H), 8.465~8.470 (m, 1 H), 8.301 (s, 1 H), 6.643-6.691 (m, 1 H), 4.955 (m, 2 H), 3.767~3.787 (m, 4 H), 3.206~3.216 (m, 4 H), 3.061 (s, 6H), 3.008 (s, 3 H) MS (ESI): 564.2 ([M + H]⁺) |
| 1.15 | 7-(5,6-Difluoro-8- (methylamino)-4- (pyrrolidin-1-yl)-9H- pyrido[2,3-b]indol-3-yl)-9- ((dimethylamino)methyl)-4- oxo-4H-pyrido[1,2- a]pyrimidine-3-carboxylic acid 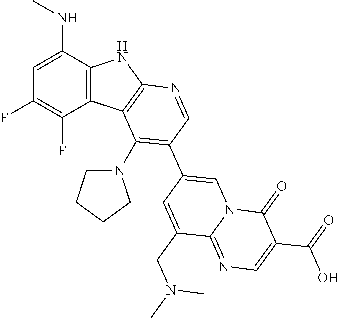 [SMILES: CNc1cc(F)c(F)c2c1[nH]c1ncc(-c3cc(CN(C)C)c4ncc(C(=O)O)c(=O)n4c3)c(N3CCCC3)c12] | CORE: Intermediate A3 AMINE: Pyrrolidine BORONIC REAGENT: Intermediate B8 CATALYST: Pd- Ad₂nBuP Biphenyl Precat (CAS: 1375477- 29-4) SOLVENT: THF/H₂O | ¹HNMR (400 MHz, DMSO-d₆) δ ppm: 11.776 (s, 1H), 9.100~9.105 (d, J = 2.0 Hz, 1H), 8.959 (s, 1H), 8.290 (s, 1H), 8.261 (s, 1H), 6.562~6.612 (m, 1H), 5.669 (s, 1H), 4.355~4.367 (d, J = 4.8 Hz, 2H), 3.211 (s, 4H), 2.889-2.895 (d, J = 2.4 Hz, 3H), 2.606 (s, 6H), 1.846 (s, 4H). MS: 548.2 ([M + H]⁺), 274.7 ([M + 2H]⁺). |
| 1.16 | 7-(6-Chloro-4- (dimethylamino)-8- (methylamino)-9H- pyrido[2,3-b]indol-3-yl)-4- oxo-4H-pyrido[1,2- a]pyrimidine-3-carboxylic acid 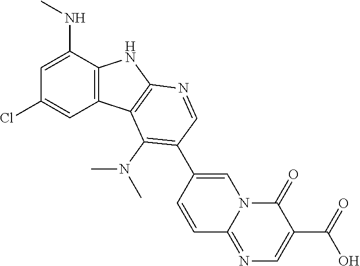 [SMILES: CNc1cc(Cl)cc2c1[nH]c1ncc(-c3ccc4ncc(C(=O)O)c(=O)n4c3)c(N(C)C)c12] | CORE: Intermediate A2 AMINE: Dimethylamine BORONIC REAGENT: Intermediate B1 CATALYST: Pd- Ad₂nBuP Biphenyl Precat (CAS: 1375477- 29-4) SOLVENT: THF/H₂O | ¹HNMR (400 MHz, MeOH-d4): δ 11.864 (s, 1 H), 9.106 (s, 1 H), 8.264~8.269 (d, J = 2.4 Hz, 1 H), 8.242~8.247 (d, J = 2.4 Hz, 1 H), 8.017(s, 1 H), 7.277 (s, 1 H), 6.599 (s, 1 H), 2.927 (s, 3 H), 2.902 (s, 6 H). MS: 463.1 ({³⁵Cl}M + H)⁺, 465.1 ({³⁷Cl}M + H)⁺. |
| 1.17 | 7-(4-(Dimethylamino)-5,7- difluoro-8-(methylamino)- 9H-pyrido[2,3-b]indol-3-yl)- 4-oxo-4H-pyrido[1,2- a]pyrimidine-3-carboxylic acid 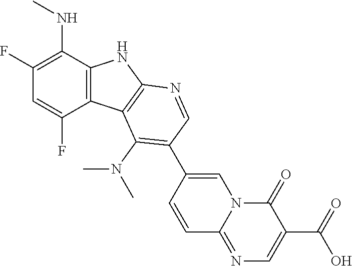 [SMILES: CNc1c(F)cc(F)c2c1[nH]c1ncc(-c3ccc4ncc(C(=O)O)c(=O)n4c3)c(N(C)C)c12] | CORE: Intermediate A5 AMINE: Dimethylamine hydrochloride BORONIC REAGENT: Intermediate B1 CATALYST: Pd- Ad₂nBuP Biphenyl Precat (CAS: 1375477- 29-4) SOLVENT: THF/H₂O | ¹HNMR (400 MHz, MeOD): δ ppm 9.388~9.393 (m, 1 H), 9.210 (s, 1 H), 8.465~8.470 (m, 1 H), 8.301 (s, 1 H), 6.643~6.691 (m, 1 H), 4.955 (m, 2 H), 3.767~3.787 (m, 4 H), 3.206~3.216 (m, 4 H), 3.061 (s, 6 H), 3.008 (s, 3 H). MS (ESI): 465.1 ([M + H]⁺) |
| 1.18 | 7-(4-(3- ((Dimethylamino)methyl)-3- fluoropyrrolidin-1-yl)-5,6- difluoro-8-(methylamino)- 9H-pyrido[2,3-b]indol-3-yl)- 4-oxo-4H-pyrido[1,2- a]pyrimidine-3-carboxylic acid 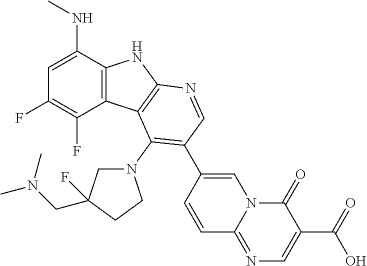 [SMILES: CNc1cc(F)c(F)c2c1[nH]c1ncc(-c3ccc4ncc(C(=O)O)c(=O)n4c3)c(N3CCC(F)(CN(C)C)C3)c12] | CORE: Intermediate A3 AMINE: C1 BORONIC REAGENT: Intermediate B1 CATALYST: Pd- Ad₂nBuP Biphenyl Precat (CAS: 1375477- 29-4) SOLVENT: THF/H₂O | ¹H NMR (400 MHz, DMSO-d6): δ 11.662 (s, 1 H), 9.438~9.443 (m, 1 H), 9.281 (s, 1 H), 8.986 (s, 1 H), 8.342 (s, 1 H), 7.250~7.280 (m, 1 H), 6.472~6.507 (m, 1 H), 4.300~4.764 (m, 1 H), 4.190 (s, 3 H), 3.642~3.670 (m, 1 H), 2.938 (s, 3 H), 2.767~2.779 (m, 2 H), 1.228~1.291 (m, 2 H), 0.636~0.671 (m, 2 H), 0.098~0.122 (m, 1 H). MS (ESI): 566.2 ([M + H]⁺) |
| 1.19 | 7-(5,6-Difluoro-4-(3-(((2- methoxyethyl)(methyl)amino) methyl)pyrrolidin-1-yl)-8- (methylamino)-9H- pyrido[2,3-b]indol-3-yl)-4- oxo-4H-pyrido[1,2- a]pyrimidine-3-carboxylic acid 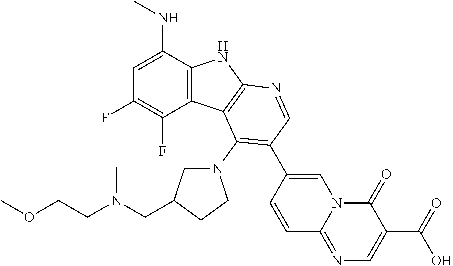 [SMILES: CNc1cc(F)c(F)c2c1[nH]c1ncc(-c3ccc4ncc(C(=O)O)c(=O)n4c3)c(N3CCC(CN(C)CCOC)C3)c12] | CORE: Intermediate A3 AMINE: Intermediate C2 BORONIC REAGENT: Intermediate B1 CATALYST: Pd- Ad₂nBuP Biphenyl Precat (CAS: 1375477- 29-4) SOLVENT: THF/H₂O | 1H NMR (400 MHz, DMSO-d₆) δ ppm: 11.719~ 11.868 (m, 1H), 9.080 (s, 1 H), 8.957 (s, 1 H), 8.209~ 8.287 (m, 2 H), 7.955 (d, J = 8.4 Hz, 1 H), 6.584 (dd, 7 = 13.6, 6.4 Hz, 1 H), 5.696 (br s, 1 H), 3.233 (br t, J = 6.0 Hz, 4 H), 3.113 (s, 3 H), 2.897 (s, 3 H), 2.802~ 2.841 (m, 1 H), 2.301~ 2.397 (m, 2 H), 2.198~ 2.288 (m, 2 H), 2.068 (s, 3 H), 1.946~1.960 (m, 1 H), 1.539 (m, 1 H). MS: 592.4 ([M + H]⁺). |
| 1.20 | 7-(4-(3-(((2,2- Difluoroethyl)(methyl)amino) methyl)pyrrolidin-1-yl)- 5,6-difluoro-8- (methylamino)-9H- pyrido [2,3-b]indol-3-yl)-4- oxo-4H-pyrido[1,2- a]pyrimidine-3-carboxylic acid 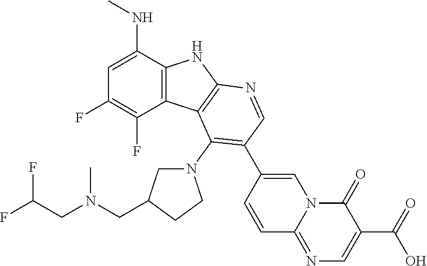 [SMILES: CNc1cc(F)c(F)c2c1[nH]c1ncc(-c3ccc4ncc(C(=O)O)c(=O)n4c3)c(N3CCC(CN(C)CC(F)F)C3)c12] | CORE: Intermediate A3 AMINE: Intermediate C3 BORONIC REAGENT: Intermediate B1 CATALYST: Pd- Ad₂nBuP Biphenyl Precat (CAS: 1375477- 29-4) SOLVENT: THF/H₂O | ¹HNMR (400 MHz, DMSO- d₆) δ: 11.821 (s, 1 H), 8.961~ 9.065 (m, 2 H), 8.231 (s, 2 H), 7.922~7.935 (m, 1 H), 6.557~6.586 (m, 1 H), 5.715~5.752 (m, 2 H), 3.238~3.241 (m, 3 H), 2.884 (s, 3 H), 2.805 (s, 1 H), 2.625~2.665 (m, 2 H), 2.382~2.402 (m, 2 H), 2.308~2.328 (m, 1 H), 2.157 (s, 3 H), 1.948 (s, 1 H), 1.528 (s, 1 H). MS: 598.1 ([M + H]⁺), 620.1 ([M + Na]⁺). |
| 1.21 | 7-(4-(3- ((cyclopropyl(methyl)amino) methyl)pyrrolidin-1-yl)-6- fluoro-8-(methylamino)-9H- pyrido[2,3-b]indol-3-yl)-4- oxo-4H-pyrido[1,2- a]pyrimidine-3-carboxylic acid 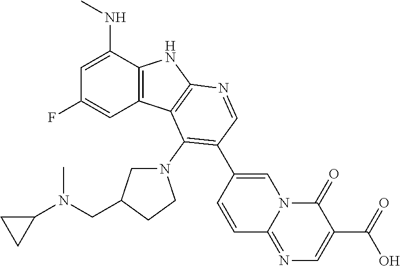 [SMILES: CNc1cc(F)cc2c1[nH]c1ncc(-c3ccc4ncc(C(=O)O)c(=O)n4c3)c(N3CCC(CN(C)C4CC4)C3)c12] | CORE: Intermediate A1 AMINE: N-methyl-N- (pyrrolidin-3- ylmethyl)cyclo- propanamine BORONIC REAGENT: Intermediate B1 CATALYST: Pd- Ad₂nBuP Biphenyl Precat (CAS: 1375477- 29-4) SOLVENT: THF/H₂O | ¹H NMR (400 MHz, DMSO- d₆) δ 11.72 (s, 1 H), 9.14 (d, J = 1.6 Hz, 1H), 8.98 (s, 1 H), 8.32 (dd, J = 2.1, 9.0 Hz, 1 H), 8.26 (s, 1 H), 7.99 (d, J = 8.9 Hz, 1 H), 6.81 (br d, J = 8.0 Hz, 1 H), 6.47 (dd, J = 2.1, 12.2 Hz, 1H), 3.33- 3.21 (m, 4 H), 3.02-2.86 (m, 5 H), 2.86-2.72 (m, 5 H), 2.26-2.13 (m, 1 H), 1.75 (br s, 1H), 0.95-0.68 (m, 4 H). MS: 556.2 ([M + H⁺), |
| 1.22 | 7-[4-[4- [(dimethylamino)methyl]- 3,3-difluoro-pyrrolidin-1- yl]-5,6-difluoro-8- (methylamino)-9H- pyrido[2,3-b]indol-3-yl]-4- oxo-pyrido[1,2- a]pyrimidine-3-carboxylic acid 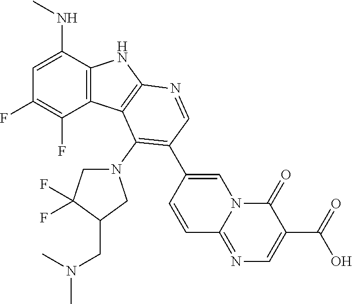 | CORE: Intermediate A3 AMINE: Intermediate C4 BORONIC REAGENT: Intermediate B21 CATALYST: Pd- Ad₂nBuP Biphenyl Precat (CAS: 1375477- 29-4) SOLVENT: THF/H₂O | ¹H NMR (400 MHz, DMSO) δ ppm 12.005 (s, 1 H), 9.530 (s, 1 H), 9.161~ 9.165 (m, 1H), 9.006 (s, 1 H), 8.328~8.381 (m, 2 H), 7.983~8.005 (m, 1 H), 6.632~6.681 (m, 1 H), 3.858 (m, 2 H), 3.437~ 3.469 (m,2H), 3.158~ 3.232 (m, 3 H), 2.916 (m, 3 H), 2.773~2.916 (m, 6 H). MS (ESI): 460.1 ([M + H]⁺). |
| 1.23 | 7-(5-Chloro-4- (dimethylamino)-6-fluoro-8- (methylamino)-9H- pyrido[2,3-b]indol-3-yl)-4- oxo-4H-pyrido[1,2- a]pyrimidine-3-carboxylic acid 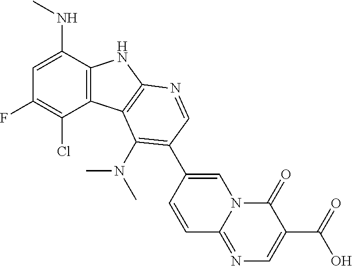 [SMILES: CNc1cc(F)c(Cl)c2c1[nH]c1ncc(-c3ccc4ncc(C(=O)O)c(=O)n4c3)c(N(C)C)c12] | CORE: Intermediate A6 AMINE: Dimethylamine hydrochloride BORONIC REAGENT: Intermediate B1 CATALYST: Pd- Ad₂nBuP Biphenyl Precat (CAS: 1375477- 29-4) SOLVENT: THF/H₂O | ¹HNMR (400 MHz, DMSO- d₆) δ: 11.88 (br s, 1 H), 9.08 (s, 1 H), 8.97 (s, 1 H), 8.18~ 8.24 (m, 2 H), 7.99 (br d, J = 8.9 Hz, 1 H), 6.61 (br d, J = 12.3 Hz, 1 H), 2.91 (s, 3 H), 2.79 (s, 6 H). MS: 481.1 ([M + H]⁺), 241.1 ([M + 2H]⁺). |
| 1.24 | 7-(5,6-Difluoro-4-((2- methoxyethyl)(methyl)amino)- 8-(methylamino)-9H- pyrido[2,3-b]indol-3-yl)-4- oxo-4H-pyrido[1,2- a]pyrimidine-3-carboxylic acid 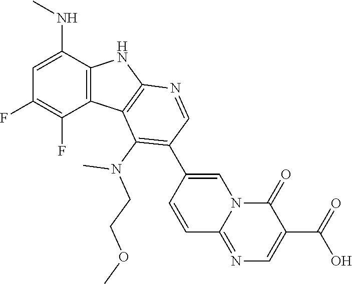 [SMILES: CNc1cc(F)c(F)c2c1[nH]c1ncc(-c3ccc4ncc(C(=O)O)c(=O)n4c3)c(N(C)CCOC)c12] | CORE: Intermediate A3 AMINE: 2-Methoxy-N- methylethanamine BORONIC REAGENT: Intermediate B1 CATALYST: Pd- Ad₂nBuP Biphenyl Precat (CAS: 1375477- 29-4) SOLVENT: THF/H₂O | ¹HNMR (400 MHz, DMSO) δ: 12.288 (s, 1 H), 9.183 (s, 1H), 8.991 (s, 1 H), 8.349 (s, 1 H), 8.254 (s, 1 H), 8.030~8.052 (m, 1 H), 6.607~6.656 (m, 1 H), 3.447~3.459 (m, 2 H), 3.350 (m, 2 H), 3.113 (s, 3 H), 2.897~2.907 (m, 6 H) MS (ESI): 509.1 [M + H]⁺).. |
| 1.25 | 7-(6-Fluoro-8- (methylamino)-4-(3- phenylpyrrolidin-1-yl)-9H- pyrido[2,3-b]indol-3-yl)-4- oxo-4H-pyrido[1,2- a]pyrimidine-3-carboxylic acid 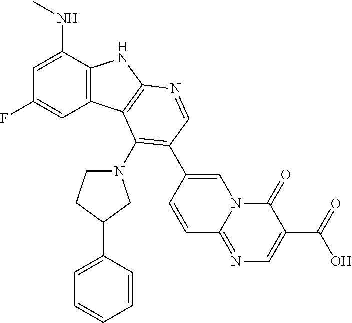 [SMILES: CNc1cc(F)cc2c1[nH]c1ncc(-c3ccc4ncc(C(=O)O)c(=O)n4c3)c(N3CCC(c4ccccc4)C3)c12] | CORE: Intermediate A1 AMINE: 3- Phenylpyrrolidine BORONIC REAGENT: Intermediate B1 CATALYST: Pd- Ad₂nBuP Biphenyl Precat (CAS: 1375477- 29-4) SOLVENT: THF/H₂O | ¹H NMR (400 MHz, DMSO-d₆) δ ppm: 11.575 (s, 1 H), 9.114 (s, 1 H), 8.979 (s, 1H), 8.315~ 8.336 (d, J = 8.4 Hz, 1 H), 8.250 (s, 1 H), 7.965~ 7.987 (d, J = 8.8 Hz, 1 H), 7.175~7.256 (m, 5 H), 6.810~6.832 (d, J = 8.8 Hz, 1H), 6.431~6.467 (m, 1 H), 5.937~5.945 (d, J = 4.8 Hz, 1 H), 3.494~3.510 (d, J = 6.4 Hz, 5 H), 2.913~ 2.924 (d, J = 4.4 Hz, 3 H), 2.368~2.397 (m, 1 H), 2.072~2.110 (m, 1H). MS (ESI): 549.1 ([M + H]⁺). |
| 1.26 | (S)-7-(4-(2- ((Dimethylamino)methyl) pyrrolidin-1-yl)-6,7-difluoro- 8-(methylamino)-9H- pyrido[2,3-b]indol-3-yl)-4- oxo-4H-pyrido[1,2- a]pyrimidine-3-carboxylic acid 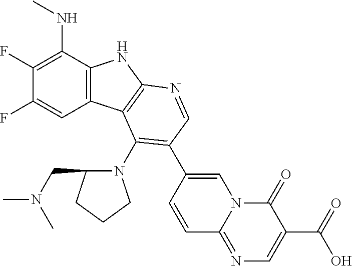 [SMILES: CNc1c(F)c(F)cc2c1[nH]c1ncc(-c3ccc4ncc(C(=O)O)c(=O)n4c3)c(N3CCC[C@H]3CN(C)C)c12] | CORE: Intermediate A4 AMINE: (S)-N,N- dimethyl-1-(pyrrolidin-2- yl)methanamine C4 BROMIDE REAGENT: Intermediate B1 CATALYST: Pd- Ad₂nBuP Biphenyl Precat (CAS: 1375477- 29-4) SOLVENT: THF/H₂O | ¹H NMR (400 MHz, Methanol-d4): δ 11.841 (s, 1 H), 9.169 (s, 1 H), 9.052 (m, 1 H), 9.001 (s, 1 H), 8.334 (s, 1 H), 8.249-8.268 (m, 1 H), 8.008~8.030 (m, 1 H), 7.029~7.072 (m, 1 H), 3.282~3.304 (m, 1 H), 3.123 (m, 3 H), 3.025 (m, 1 H), 3.769~3.781 (m, 2 H), 2.571~2.582 (m, 1 H), 2.412 (s, 3 H), 2.401 (s, 3 H), 2.030 (m, 2 H), 1.843 (m, 2 H). MS (ESI): 548.1([M + H]⁺). |
| 1.27 | (R)-7-(4-(3- (Dimethylamino)pyrrolidin- 1-yl)-6,7-difluoro-8- (methylamino)-9H- pyrido[2,3-b]indol-3-yl)-4- oxo-4H-pyrido[1,2- a]pyrimidine-3-carboxylic acid 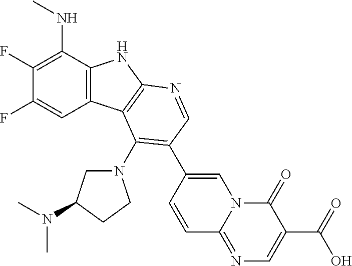 | CORE: Intermediate A7 AMINE: (R)-N,N- dimethylpyrrolidin-3- amine BORONIC REAGENT: Intermediate B1 CATALYST: Pd- Ad₂nBuP Biphenyl Precat (CAS: 1375477- 29-4) SOLVENT: THF/H₂O | ¹H NMR (400 MHz, MeOD-d4) δ 11.856 (s, 1 H), 9.900 (s, 2 H), 9.119 (s, 1 H), 9.003 (s, 1 H), 8.294~ 8.318 (m, 1 H), 7.957~ 7.980 (m, 1 H), 7.040~ 7.084 (m, 1 H), 3.973~ 3.991 (m, 1 H), 3.170~ 3.190 (m, 3H), 3.127~ 3.138 (m, 1 H), 2.820 (m, 3 H), 2.687 (m, 3 H), 2.415~ 2.449 (m, 1 H), 2.117~ 2.149 (m, 1 H). MS (ESI): 521.1([M+H]+). |
| 1.28 | 7-(5,6-Difluoro-4- (hexahydropyrazino[2,1- c][1,4]oxazin-8(1H)-yl)-8- (methylamino)-9H- pyrido[2,3-b]indol-3-yl)-4- oxo-4H-pyrido[1,2- a]pyrimidine-3-carboxylic acid compound with formic acid 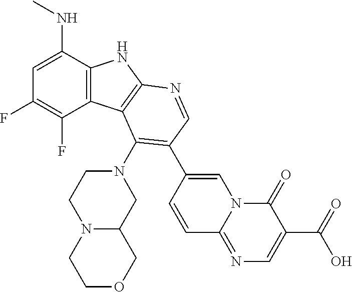 [SMILES: CNc1cc(F)c(F)c2c1[nH]c1ncc(-c3ccc4ncc(C(=O)O)c(=O)n4c3)c(N3CCN4CCOCC4C3)c12] | CORE: Intermediate A3 AMINE: Octahydropyrazino[2,1- c][1,4]oxazine BORONIC REAGENT: Intermediate B1 CATALYST: Pd- Ad₂nBuP Biphenyl Precat (CAS: 1375477- 29-4) SOLVENT: THF/H₂O | ¹H NMR (400 MHz, DMSO- d₆) δ 12.17 (br s, 1 H), 9.04 (br s, 1 H), 8.43 (s, 3H), 8.17 (br s, 2 H), 7.89 (br s, 1 H), 6.60 (dd, J = 5.8, 13.5 Hz, 1H), 5.91 (br s, 1 H), 3.65 (br s, 1 H), 3.58-3.51 (m, 3 H), 2.89 (br d, J = 4.3 Hz, 6 H), 2.63-2.57 (m, 3 H), 2.29-2.09 (m, 3 H) MS (ESI): 562.0 ([M + H]⁺) |
| 1.29 | 7-[8-(Ethylamino)-6-fluoro- 4-[3- (trifluoromethyl)pyrazol-1- yl]-9H-pyrido[2,3-b]indol-3- yl]-9-methyl-4-oxo- pyrido[1,2-a]pyrimidine-3- carboxylic acid 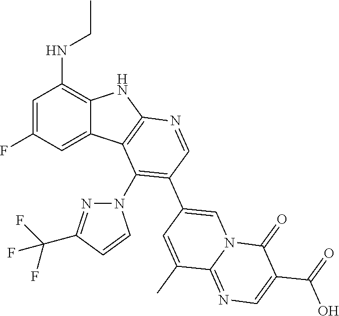 [SMILES: CCNc1cc(F)cc2c1[nH]c1ncc(-c3cc(C)c4ncc(C(=O)O)c(=O)n4c3)c(-n3ccc(C(F)(F)F)n3)c12] | CORE: Intermediate A7 AMINE: 3- (Trifluoromethyl)-1H- pyrazole BORONIC REAGENT: Intermediate B2 CATALYST: Xphos Pd G2(CAS: 1310584-14-5) SOLVENT: THF/H₂O | ¹H NMR (METHANOL-d₄, 400 MHz) δ 9.0-9.1 (m, 1H), 8.8-8.9 (m, 1H), 8.6- 8.6 (m, 1H), 8.4-8.5 (m, 1H), 7.9-8.0 (m, 1H), 7.3- 7.4 (m, 1H), 6.8-6.8 (m, 1H), 6.3-6.4 (m, 1H), 5.8- 5.9 (m, 1H), 3.2-3.3 (m, 2H), 2.4-2.4 (m, 3H), 1.3- 1.3 (m, 3H) MS: 565.9 ([M + H]⁺) |
| 1.30 | 7-[8-(Ethylamino)-6-fluoro- 4- [3- (trifluoromethyl)pyrazol-1- yl]-9H-pyrido[2,3-b]indol-3- yl]-4-oxo-pyrido[1,2- a]pyrimidine-3-carboxylic acid 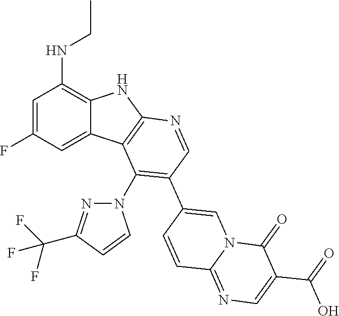 [SMILES: CCNc1cc(F)cc2c1[nH]c1ncc(-c3ccc4ncc(C(=O)O)c(=O)n4c3)c(-n3ccc(C(F)(F)F)n3)c12] | CORE: Intermediate A7 AMINE: 3- (Trifluoromethyl)-1H- pyrazole BORONIC REAGENT: Intermediate B1 CATALYST: Xphos Pd G2(CAS: 1310584-14-5) SOLVENT: THF/H₂O | ¹H NMR (METHANOL-d4, 400 MHz) δ 9.1-9.1 (m, 1H), 8.6-8.6 (m, 1H), 8.0- 8.0 (m, 1H), 7.4-7.5 (m, 2H), 6.81 (s, 1H), 6.4-6.4 (m, 2H), 5.9-5.9 (m, 2H), 3.0-3.1 (m, 2H), 1.3-1.4 (m, 3H) MS: 552.0 ([M + H]⁺) |
| 1.31 | 8-[8-(Ethylamino)-6-fluoro- 4-[3- (trifluoromethyl)pyrazol-1- yl]-9H-pyrido[2,3-b]indol-3- yl]-4-oxo-pyrido[1,2- a]pyrimidine-3-carboxylic acid 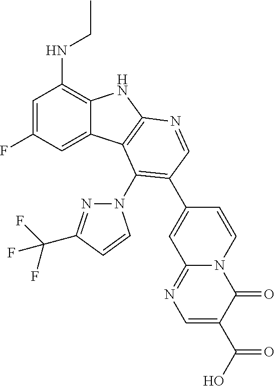 [SMILES: CCNc1cc(F)cc2c1[nH]c1ncc(-c3ccn4c(=O)c(C(=O)O)cnc4c3)c(-n3ccc(C(F)(F)F)n3)c12] | CORE: Intermediate A7 AMINE: 3- (Trifluoromethyl)-1H- pyrazole BORONIC REAGENT: Intermediate B1 CATALYST: Xphos Pd G2(CAS: 1310584-14-5) SOLVENT: THF/H₂O | ¹H NMR (DMSO-d₆, 400 MHz) δ 9.0-9.1 (m, 1H), 8.9-9.0 (m, 2H), 8.4-8.4 (m, 1H), 7.7-7.7 (m, 1H), 7.2- 7.2 (m, 1H), 7.1-7.2 (m, 2H), 6.6-6.7 (m, 1H), 6.5- 6.5 (m, 1H), 3.3-3.3 (m, 2H), 1.3-1.4 (m, 3H) MS: 552.1 ([M + H]⁺) |
| 1.32 | 7-[4-(Dimethylamino)-5,6- difluoro-8-(methylamino)- 9H-pyrido[2,3-b]indol-3-yl]- 9-morpholino-4-oxo- pyrido[1,2-a]pyrimidine-3- carboxylic acid 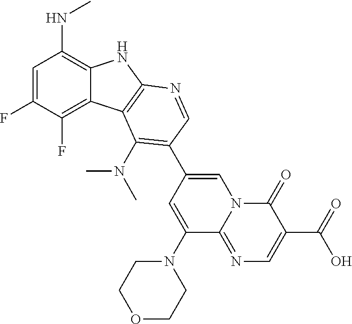 [SMILES: CNc1cc(F)c(F)c2c1[nH]c1ncc(-c3cc(N4CCOCC4)c4ncc(C(=O)O)c(=O)n4c3)c(N(C)C)c12] | CORE: Intermediate A3 AMINE: N- methylmethanamine; hydrochloride BORONIC REAGENT: Intermediate B4 CATALYST: Xphos Pd G2(CAS: 1310584-14-5) SOLVENT: THF/H₂O | ¹H NMR (DMSO-d₆, 400 MHz) δ 11.8-11.8 (m, 1H), 8.9-9.0 (m, 1H), 8.7-8.8 (m, 1H), 8.2-8.3 (m, 1H), 7.5- 7.5 (m, 1H), 6.5-6.7 (m, 1H), 3.8-3.9 (m, 4H), 3.3- 3.4 (m, 4H), 2.9-2.9 (m, 3H), 2.8-2.8 (m, 6H) MS: 550.1 ([M + H]⁺) |
| 1.33 | 7-[5,6-Difluoro-4-(5-methyl- 2,3,3a,4,6,6a- hexahydropyrrolo[2,3- c]pyrrol-1-yl)-8- (methylamino)-9H- pyrido[2,3-b]indol-3-yl]-9- methyl-4-oxo-pyrido[1,2- a]pyrimidine-3-carboxylic acid 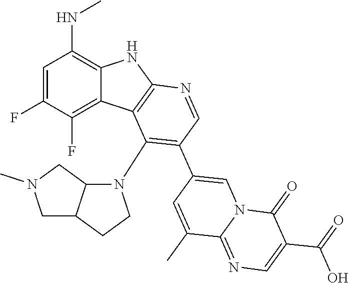 [SMILES: CNc1cc(F)c(F)c2c1[nH]c1ncc(-c3cc(C)c4ncc(C(=O)O)c(=O)n4c3)c(N3CCC4CN(C)CC43)c12] | CORE: Intermediate A3 AMINE: 3- (Trifluoromethyl)-1H- pyrazole BORONIC REAGENT: Intermediate B2 CATALYST: Xphos Pd G2(CAS: 1310584-14-5) SOLVENT: THF/H₂O | ¹H NMR (DMSO-d₆, 400 MHz) δ 8.8-8 9 (m, 2H), 8.3-8.3 (m, 1H), 8.1-8.2 (m, 1H), 6.5-6.6 (m, 1H), 5.6- 5.7 (m, 1H), 4.4-4.5 (m, 1H), 2.9-3.0 (m, 2H), 2.8- 2.9 (m, 3H), 2.6-2.6 (m, 1H), 2.5-2.6 (m, 3H), 2.5- 2.5 (m, 2H), 2.2-2.3 (m, 1H), 2.1-2.1 (m, 1H), 2.1- 2.1 (m, 3H), 1.9-2.0 (m, 1H), 1.7-1.7 (m, 1H), 1.5- 1.6 (m, 1H) MS: 560.4 ([M + H]⁺) |
| 1.34 | 7-[4-Cyano-6-fluoro-8- (methylamino)-9H- pyrido[2,3-b]indol-3-yl]-4- oxo-pyrido[1,2- a]pyrimidine-3-carboxylic acid 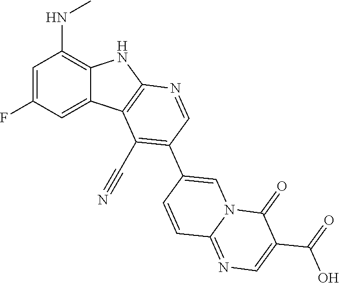 [SMILES: CNc1cc(F)cc2c1[nH]c1ncc(-c3ccc4ncc(C(=O)O)c(=O)n4c3)c(C#N)c12] | CORE: Intermediate A1 NUCLEOPHILE: Dicyanozinc BORONIC REAGENT: Intermediate B1 CATALYST: Xphos Pd G2(CAS: 1310584-14-5) SOLVENT: THF/H₂O | ¹H NMR (DMSO-d₆, 400 MHz) δ 9.4-9.5 (m, 1H), 8.9-9.0 (m, 1H), 8.8-8.9 (m, 1H), 8.5-8.6 (m, 1H), 8.0- 8.1 (m, 1H), 7.2-7.3 (m, 1H), 6.6-6.7 (m, 1H), 6.53 (m, 1H), 6.0-6.2 (m, 1H), 2.9-3.0 (m, 3H) MS: 429.4 ([M + H]⁺) |

### Example 2.01

### 7-[4-(Dimethylamino)-5,6-difluoro-8-(methylamino)-9H-pyrido[2,3-b]indol-3-yl]-9-(morpholinomethyl)-4-oxo-pyrido[1,2-a]pyrimidine-3-carboxylic Acid

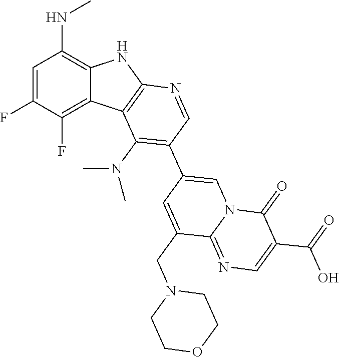 [SMILES: CNc1cc(F)c(F)c2c1[nH]c1ncc(-c3cc(CN4CCOCC4)c4ncc(C(=O)O)c(=O)n4c3)c(N(C)C)c12]

### Step (a) Preparation of Tert-Butyl N-[3-bromo-4-(dimethylamino)-5,6-difluoro-9H-pyrido[2,3-b]indol-8-yl]-N-methyl-carbamate

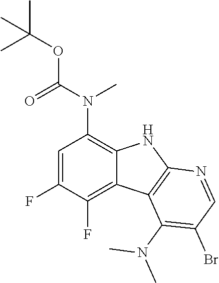 [SMILES: CN(C)c1c(Br)cnc2[nH]c3c(N(C)C(=O)OC(C)(C)C)cc(F)c(F)c3c12]

The solution of tert-butyl N-(3-bromo-4-chloro-5,6-difluoro-9H-pyrido[2,3-b]indol-8-yl)-N-methyl-carbamate (Intermediate A3, 1 g, 2.24 mmol, as the “CORE” in Table 2), N-methylmethanamine hydrochloride (730 mg, 8.96 mmol, as the “AMINE” in Table 2) and TEA (1.13 g, 11.20 mmol) was stirred at 110° C. for 18 h under N₂ atmosphere. After the reaction mixture was cooled back to room temperature and poured into water (80 mL), it was extracted with EtOAc (80 mL) three times. The combined organics were washed with brine (100 mL), dried over anhy. Na₂SO₄, filtered, and concentrated in vacuo. The residue was purified by prep-HPLC to give tert-butyl N-[3-bromo-4-(dimethylamino)-5,6-difluoro-9H-pyrido[2,3-b]indol-8-yl]-N-methyl-carbamate (536 mg, 52.6% yield) as a white solid. MS (ESI): 455.2 ([{³⁵Cl} M+H]⁺), 457.2 ([{³⁷Cl} M+H]⁺).

### Step (b) Preparation of [8-[tert-butoxycarbonyl(methyl)amino]-4-(dimethylamino)-5,6-difluoro-9H-pyrido[2,3-b]indol-3-yl]boronic Acid

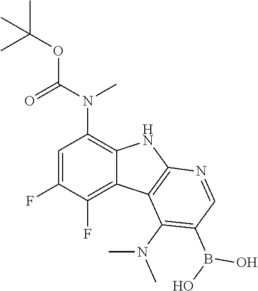 [SMILES: CN(C)c1c(B(O)O)cnc2[nH]c3c(N(C)C(=O)OC(C)(C)C)cc(F)c(F)c3c12]

To a stirred solution of tert-butyl (3-bromo-4-(dimethylamino)-5,6-difluoro-9H-pyrido[2,3-b]indol-8-yl)(methyl)carbamate (0.3 g, 659 μmol) in THF (10 mL) was added slowly magnesium chloride propan-2-ide-LiCl (5.07 mL, 6.59 mmol) dropwise at 0° C. After the addition, the mixture was allowed to warm up and stirred at room temperature for 3 h. 2-Isopropoxy-4,4,5,5-tetramethyl-1,3,2-dioxaborolane (613 mg, 3.29 mmol) was then added and the resulting mixture was stirred at r.t. overnight. The mixture was diluted with EtOAc (50 mL), washed with aq. NH₄Cl solution (20 mL). The separated organic layer was washed with brine, dried over anhy. Na₂SO₄, filtered, and concentrated in vacuo to give a crude product of [8-[tert-butoxycarbonyl(methyl)amino]-4-(dimethylamino)-5,6-difluoro-9H-pyrido[2,3-b]indol-3-yl]boronic acid. MS (ESI): 421.1 ([M+H]⁺). It was used directly in the next step without further purification.

### Step (c) Preparation of Ethyl 7-[8-[tert-butoxycarbonyl(methyl)amino]-4-(dimethylamino)-5,6-difluoro-9H-pyrido[2,3-b]indol-3-yl]-9-(morpholinomethyl)-4-oxo-pyrido[1,2-a]pyrimidine-3-carboxylate

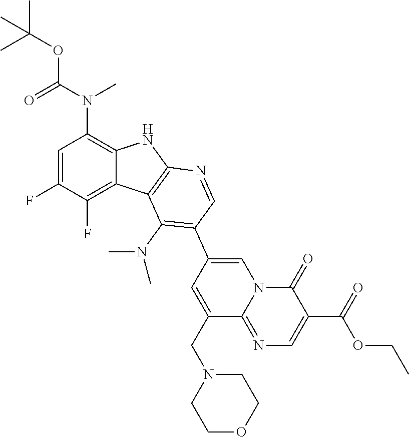 [SMILES: CCOC(=O)c1cnc2c(CN3CCOCC3)cc(-c3cnc4[nH]c5c(N(C)C(=O)OC(C)(C)C)cc(F)c(F)c5c4c3N(C)C)cn2c1=O]

To a stirred solution of (8-((tert-butoxycarbonyl)(methyl)amino)-4-(dimethylamino)-5,6-difluoro-9H-pyrido[2,3-b]indol-3-yl)boronic acid (0.277 g, 659 μmol) and ethyl 7-bromo-9-(morpholinomethyl)-4-oxo-4H-pyrido[1,2-a]pyrimidine-3-carboxylate (261 mg, 659 μmol) in THF (3 mL) and water (300 μl) were added Pd-Adn-nBuP Biphenyl G2 (156 mg, 198 μmol, CAS: 1375477-29-4) and K₃PO₄ (420 mg, 1.98 mmol) under glove box. The mixture was first stirred at room temperature for 10 min and then heated at 60° C. overnight. After the reaction was cooled back to r.t., the mixture was diluted with EtOAc (40 mL) and washed with water (20 mL). The organic was dried over anhy. Na₂SO₄, filtered, and concentrated in vacuo to give a crude product, which was further purified by prep-HPLC to give

### Ethyl 7-[8-[tert-butoxycarbonyl(methyl)amino]-4-(dimethylamino)-5,6-difluoro-9H-pyrido[2,3-b]indol-3-yl]-9-(morpholinomethyl)-4-oxo-pyrido[1,2-a]pyrimidine-3-carboxylate

(0.216 g, 47.4% yield) as a white solid. MS (ESI): 692.6 ([M+H]⁺).

### Step (d) Preparation of 7-[4-(dimethylamino)-5,6-difluoro-8-(methylamino)-9H-pyrido[2,3-b]indol-3-yl]-9-(morpholinomethyl)-4-oxo-pyrido[1,2-a]pyrimidine-3-carboxylic Acid

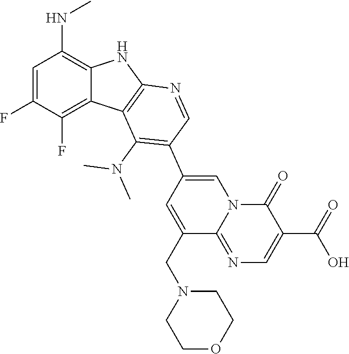 [SMILES: CNc1cc(F)c(F)c2c1[nH]c1ncc(-c3cc(CN4CCOCC4)c4ncc(C(=O)O)c(=O)n4c3)c(N(C)C)c12]

A solution of 7-(8-((tert-butoxycarbonyl)(methyl)amino)-4-(dimethylamino)-5,6-difluoro-9H-pyrido[2,3-b]indol-3-yl)-9-(morpholinomethyl)-4-oxo-4H-pyrido[1,2-a]pyrimidine-3-carboxylic acid (0.207 g, 312 μmol) and TFA (3 g, 2 mL, 26.3 mmol) in DCM (3 mL) was stirred at room temperature for 1 h before concentrated in vacuo. The residue was purified by prep-HPLC to give 7-[4-(dimethylamino)-5,6-difluoro-8-(methylamino)-9H-pyrido[2,3-b]indol-3-yl]-9-(morpholinomethyl)-4-oxo-pyrido[1,2-a]pyrimidine-3-carboxylic acid (20 mg, 11.4% yield) as a white solid. ¹H NMR (DMSO-d6, 400 MHz) δ 11.6-11.8 (m, 1H), 8.8-9.0 (m, 2H), 8.0-8.2 (m, 2H), 6.52 (dd, 1H, J=6.2, 13.5 Hz), 5.5-5.7 (m, 1H), 3.9-4.0 (m, 2H), 3.5-3.6 (m, 4H), 2.8-2.9 (m, 3H), 2.7-2.8 (m, 6H), 2.5-2.5 (m, 4H). MS (ESI): 564.3 ([M+H]⁺).

The following examples were prepared in analogy to Example 2.01, by replacing tert-butyl N-(3,4-dichloro-5,6-difluoro-9H-pyrido[2,3-b]indol-8-yl)-N-methyl-carbamate (Intermediate A3) with the “CORE” in step (a), N-methylmethanamine hydrochloride with the “AMINE” in step (a), ethyl 7-bromo-9-(morpholinomethyl)-4-oxo-4H-pyrido[1,2-a]pyrimidine-3-carboxylate with the “BROMIDE” in step (c), and XPhos Pd G2 with the “CATALYST” in step (c) by the reagents indicated in Table 2.

TABLE 2
Compound synthesis and characterization

| | | CORE, AMINE, | |
|---|---|---|---|
| | | BROMIDE | |
| | | REAGENT, | |
| | Compound Name and | CATALYST, and | |
| No. | Structure | SLOVENT | ¹H NMR and MS (ESI) |
| 2.02 | 7-[4-(Dimethylamino)-5,6- difluoro-8-(methylamino)- 9H-pyrido[2,3-b]indol-3-yl]- 9-[[2- methoxyethyl(methyl)amino] methyl]-4-oxo-pyrido[1,2- a]pyrimidine-3-carboxylic acid 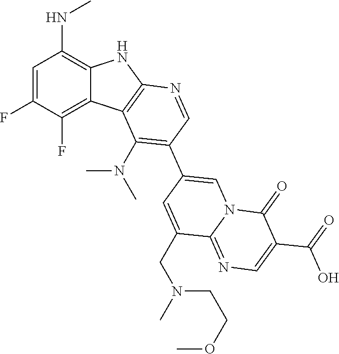 [SMILES: CNc1cc(F)c(F)c2c1[nH]c1ncc(-c3cc(CN(C)CCOC)c4ncc(C(=O)O)c(=O)n4c3)c(N(C)C)c12] | CORE: Intermediate A3 AMINE: N- methylmethanamine hydrochloride BROMIDE REAGENT: Intermediate B6 CATALYST: Pd-Adn- nBuP Biphenyl G2 (CAS: 1375477-29-4) SOLVENT: THF/H₂O | ¹H NMR (DMSO-d₆, 400 MHz) δ 11.7-11.9 (m, 1H), 9.1-9.2 (m, 1H), 8.9-9.0 (m, 1H), 8.4-8.4 (m, 1H), 8.2- 8.3 (m, 1H), 6.5-6.7 (m, 1H), 4.9-5.0 (m, 1H), 4.6- 4.8 (m, 1H), 3.8-3.8 (m, 4H), 3.3-3.4 (m, 3H), 2.9- 2.9 (m, 3H), 2.8-2 9 (m, 9H) MS: 566.0 ([M + H]⁺) |
| 2.03 | 7-[4-(Dimethylamino)-5,6- difluoro-8-(methylamino)- 9H-pyrido[2,3-b]indol-3-yl]- 4-oxo-9-(pyrrolidin-1- ylmethyl)pyrido[1,2- a]pyrimidine-3-carboxylic acid 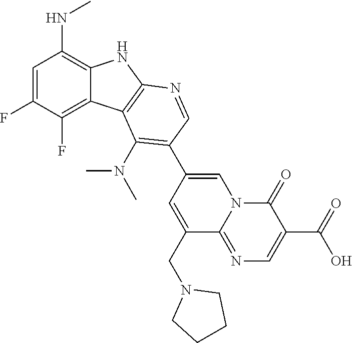 [SMILES: CNc1cc(F)c(F)c2c1[nH]c1ncc(-c3cc(CN4CCCC4)c4ncc(C(=O)O)c(=O)n4c3)c(N(C)C)c12] | CORE: Intermediate A3 AMINE: N- methylmethanamine hydrochloride BROMIDE REAGENT: Intermediate B7 CATALYST: Pd-Adn- nBuP Biphenyl G2 (CAS: 1375477-29-4) SOLVENT: THF/H₂O | ¹H NMR (DMSO-d₆, 400 MHz) δ 11.7-11.9 (m, 1H), 9.1-9.2 (m, 1H), 8.9-9.0 (m, 1H), 8.3-8.5 (m, 1H), 8.2- 8.3 (m, 1H), 6.5-6 7 (m, 1H), 4.8-4.9 (m, 2H), 3.5- 3.6 (m, 2H), 2.9-2.9 (m, 3H), 2.85 (d, 6H, J = 2.1 Hz), 2.6-2.7 (m, 1H), 2.33 (tq, 1H, J = 2.3, 4.2 Hz), 2.07 (br d, 2H, J = 1.0 Hz), 1.9-2.0 (m, 2H) MS: 548.0 ([M + H]⁺) |
| 2.04 | 7-[5,6-Difluoro-8- (methylamino)-4- pyrrolidin-1-yl-9H- pyrido[2,3-b]indol-3-yl]-9- (morpholinomethyl)-4-oxo- pyrido[1,2-a]pyrimidine-3- carboxylic acid 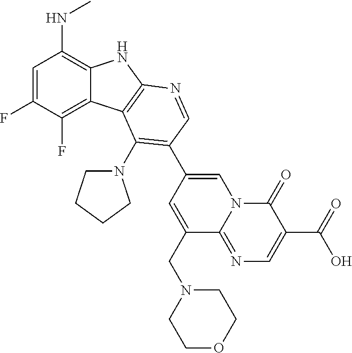 [SMILES: CNc1cc(F)c(F)c2c1[nH]c1ncc(-c3cc(CN4CCOCC4)c4ncc(C(=O)O)c(=O)n4c3)c(N3CCCC3)c12] | CORE: Intermediate A3 AMINE: Pyrrolidine BROMIDE REAGENT: Intermediate B5 CATALYST: Pd-Adn- nBuP Biphenyl G2 (CAS: 1375477-29-4) SOLVENT: THF/H₂O | ¹H NMR (METHANOL-d₄, 400 MHz) δ 12.7-12.8 (m, 1H), 10.0-10.1 (m, 1H), 9.8- 9.9 (m, 1H), 9.2-9.3 (m, 1H), 9.0-9.1 (m, 1H), 8.2- 8.3 (m, 1H), 7.4-7.5 (m, 1H), 5.64 (br s, 2H), 4.3-4.5 (m, 2H), 4.11 (br s, 8H), 3.7-3.7 (m, 3H), 2.6-2.7 (m, 4H) MS: 589.8 ([M + H]⁺) |
| 2.05 | 7-(4-(3,3- Difluoropyrrolidin-1-yl)-6- fluoro-8-(methylamino)-9H- pyrido[2,3-b]indol-3-yl)-4- oxo-4H-pyrido[1,2- a]pyrimidine-3-carboxylic acid 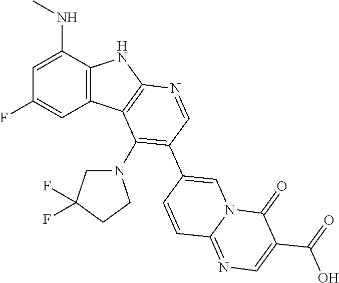 [SMILES: CNc1cc(F)cc2c1[nH]c1ncc(-c3ccc4ncc(C(=O)O)c(=O)n4c3)c(N3CCC(F)(F)C3)c12] | CORF: Intermediate A1 AMINE 3,3- Difluoropyrrolidine BROMIDE REAGENT: Intermediate B1.3 CATALYST: Pd-Adn- nBuP Biphenyl G2 (CAS: 1375477-29-4) SOLVENT: THF/H₂O | ¹H NMR (400 MHz, DMSO-d6) δ: 11.698 (s, 1 H), 9.130~9.134 (d, J = 2.0 Hz, 1 H), 8.987 (s, 1 H), 8.328~8.356 (dd, J = 8.8, 2.0 Hz, 1 H), 8.276 (s, 1 H), 7.966~7.989 (d, J = 8.8 Hz, 1 H), 6.856~6.879 (dd, J = 9.2, 2.0 Hz, 1 H), 6.465~ 6.501 (dd, J = 12.4, 2.0 Hz, 1 H), 3.593~3.656 (br t, J = 12.4 Hz, 2 H), 3.454- 3.489 (t, J = 7.2 Hz, 2 H), 2.933 (s, 3 H), 2.438~2.476 (m, 2 H). MS (ESI): 509.0 (M + H)⁺, 255.1(M/2 + H)⁺. |
| 2.06 | 7-[4-(Dimethylamino)-5,6- difluoro-8-(methylamino)- 9H-pyrido[2,3-b]indol-3-yl]- 9-[(5-methyl-2,3,3a,4,6,6a- hexahydropyrrolo[2,3- c]pyrrol-1-yl)methyl]-4-oxo- pyrido[1,2-a]pyrimidine-3- carboxylic acid 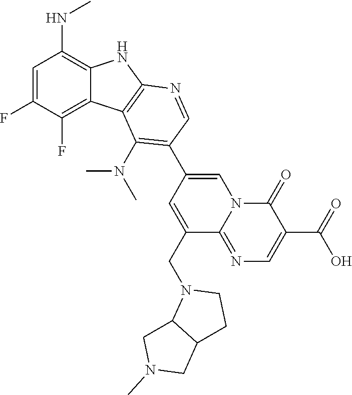 [SMILES: CNc1cc(F)c(F)c2c1[nH]c1ncc(-c3cc(CN4CCC5CN(C)CC54)c4ncc(C(=O)O)c(=O)n4c3)c(N(C)C)c12] | CORE: Intermediate A3 AMINE: N- methylmethanamine hydrochloride BROMIDE REAGENT: Intermediate B9 CATALYST: Pd-Adn- nBuP Biphenyl G2 (CAS: 1375477-29-4) | ¹H NMR (DMSO-d₆, 400 MHz) δ 11.7-11.9 (m, 1H), 9.0-9.1 (m, 2H), 8.26 (s, 2H), 7.04 (s, 1H), 6.5-6.7 (m, 1H), 4.3-4 4 (m, 1H), 4.1-4.2 (m, 1H), 3.0-3.2 (m, 6H), 2.9-2.9 (m, 3H), 2.8- 2.8 (m, 9H), 2.7-2.7 (m, 1H), 2.3-2.4 (m, 1H), 2.0- 2.2 (m, 1H), 1.5-1.7 (m, 1H) MS: 602.8 ([M + H]⁺) |

### Example 3.01

### 7-[4-Cyclopropyl-5,6-difluoro-8-(methylamino)-9H-pyrido[2,3-b]indol-3-yl]-9-methyl-4-oxo-pyrido[1,2-a]pyrimidine-3-carboxylic Acid

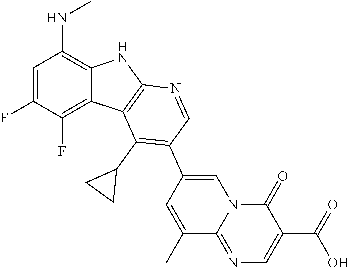 [SMILES: CNc1cc(F)c(F)c2c1[nH]c1ncc(-c3cc(C)c4ncc(C(=O)O)c(=O)n4c3)c(C3CC3)c12]

### Step (a) Preparation of Tert-Butyl N-(3-chloro-4-cyclopropyl-5,6-difluoro-9H-pyrido[2,3-b]indol-8-yl)-N-methyl-carbamate

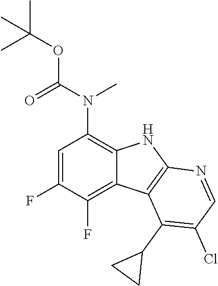 [SMILES: CN(C(=O)OC(C)(C)C)c1cc(F)c(F)c2c1[nH]c1ncc(Cl)c(C3CC3)c12]

A solution of tert-butyl (3,4-dichloro-5,6-difluoro-9H-pyrido[2,3-b]indol-8-yl)(methyl)carbamate (0.413 g, 1.03 mmol), cyclopropylboronic acid (132 mg, 1.54 mmol), tricyclohexylphosphonium tetrafluoroborate (378 mg, 1.03 mmol), and potassium phosphate (654 mg, 3.08 mmol) in toluene (24 mL) were degassed and purged with N₂ five times, then the resulting mixture was heated at 110° C. overnight. After the reaction was cooled back to r.t., it was concentrated under reduced pressure to give a crude product, which was purified by prep-HPLC to give tert-butyl N-(3-chloro-4-cyclopropyl-5,6-difluoro-9H-pyrido[2,3-b]indol-8-yl)-N-methyl-carbamate (0.241 g, 57.5% yield) a white solid. MS: 408.0 ([M+H]⁺).

### Step (b) Preparation of ethyl 7-[8-[tert-butoxycarbonyl(methyl)amino]-4-cyclopropyl-5,6-difluoro-9H-pyrido[2,3-b]indol-3-yl]-9-methyl-4-oxo-pyrido[1,2-a]pyrimidine-3-carboxylate

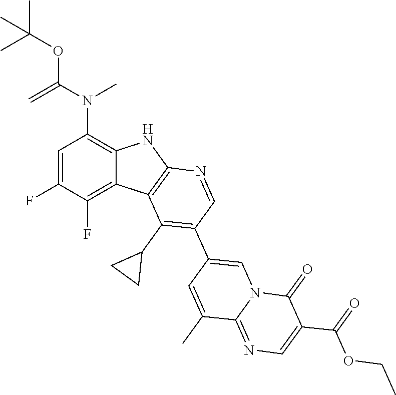 [SMILES: C=C(OC(C)(C)C)N(C)c1cc(F)c(F)c2c1[nH]c1ncc(-c3cc(C)c4ncc(C(=O)OCC)c(=O)n4c3)c(C3CC3)c12]

To a stirred solution of tert-butyl (3-chloro-4-cyclopropyl-5,6-difluoro-9H-pyrido[2,3-b]indol-8-yl)(methyl)carbamate (0.101 g, 248 μmol) and (3-(ethoxycarbonyl)-9-methyl-4-oxo-4H-pyrido[1,2-a]pyrimidin-7-yl)boronic acid (82 mg, 297 μmol) in THF (5.3 mL) were added Xphos Pd G2 (39 mg, 49.5 μmol, CAS: 1310584-14-5), and potassium phosphate (105 mg, 495 μmol) at room temperature. The mixture was degassed and purged with argon five times, and the mixture was heated at 80° C. overnight. After cooled back to r.t., the reaction mixture was concentrated in vacuo. The residue was diluted with EtOAc (50 mL) and washed with water (20 mL). The organic layer was dried over anhy. Na₂SO₄, filtered, and concentrated in vacuo to give a crude product, which was further purified by prep-HPLC to give ethyl 7-[8-[tert-butoxycarbonyl(methyl)amino]-4-cyclopropyl-5,6-difluoro-9H-pyrido[2,3-b]indol-3-yl]-9-methyl-4-oxo-pyrido[1,2-a]pyrimidine-3-carboxylate (0.063 g, 42.1% yield) as a white solid. MS: 604.3 ([M+H]⁺).

### Step (c) Preparation of 7-[8-[tert-butoxycarbonyl(methyl)amino]-4-cyclopropyl-5,6-difluoro-9H-pyrido[2,3-b]indol-3-yl]-9-methyl-4-oxo-pyrido[1,2-a]pyrimidine-3-carboxylic Acid

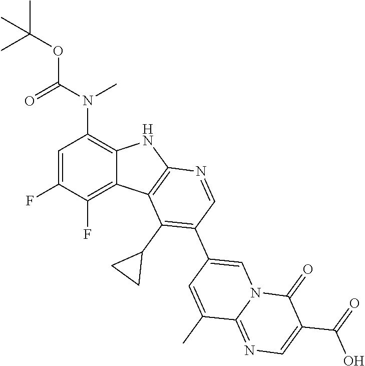 [SMILES: Cc1cc(-c2cnc3[nH]c4c(N(C)C(=O)OC(C)(C)C)cc(F)c(F)c4c3c2C2CC2)cn2c(=O)c(C(=O)O)cnc12]

To a stirred solution of ethyl 7-(8-((tert-butoxycarbonyl)(methyl)amino)-4-cyclopropyl-5,6-difluoro-9H-pyrido[2,3-b]indol-3-yl)-9-methyl-4-oxo-4H-pyrido[1,2-a]pyrimidine-3-carboxylate (0.063 g, 104 μmol) in THF (3 mL) was added sodium hydroxide (83.5 mg, 2.09 mmol) in water (3 mL), and the resulting mixture was stirred at room temperature for 18 h. The mixture was then concentrated in vacuo to give a residue, which was re-diluted with EtOAc (50 mL) and washed with water (20 mL). The separated organic layer was dried with anhy. Na₂SO₄, filtered, and concentrated in vacuo to give a crude product of 7-[8-[tert-butoxycarbonyl(methyl)amino]-4-cyclopropyl-5,6-difluoro-9H-pyrido[2,3-b]indol-3-yl]-9-methyl-4-oxo-pyrido[1,2-a]pyrimidine-3-carboxylic acid (60 mg). MS: 576.2 ([M+H]⁺). It was used directly in the next step without further purification.

### Step (d) Preparation of 7-[4-cyclopropyl-5,6-difluoro-8-(methylamino)-9H-pyrido[2,3-b]indol-3-yl]-9-methyl-4-oxo-pyrido[1,2-a]pyrimidine-3-carboxylic Acid

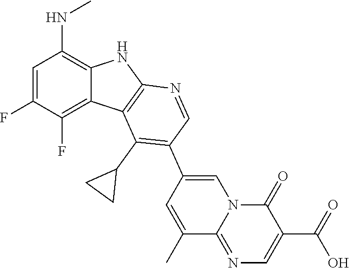 [SMILES: CNc1cc(F)c(F)c2c1[nH]c1ncc(-c3cc(C)c4ncc(C(=O)O)c(=O)n4c3)c(C3CC3)c12]

To a stirred solution of 7-(8-((tert-butoxycarbonyl)(methyl)amino)-4-cyclopropyl-5,6-difluoro-9H-pyrido[2,3-b]indol-3-yl)-9-methyl-4-oxo-4H-pyrido[1,2-a]pyrimidine-3-carboxylic acid (0.06 g, 104 μmol) in DCM (3 mL) was added TFA (3 g, 2 mL, 26.3 mmol), and the resulting mixture was stirred at room temperature for 2 h. The reaction mixture was then concentrated in vacuo to give a crude product, which was purified by prep-HPLC to give 7-[4-cyclopropyl-5,6-difluoro-8-(methylamino)-9H-pyrido[2,3-b]indol-3-yl]-9-methyl-4-oxo-pyrido[1,2-a]pyrimidine-3-carboxylic acid (8 mg, 16.1% yield) as a white solid. ¹H NMR (DMSO-d6, 400 MHz) δ 11.7-11.9 (m, 1H), 9.1-9.1 (m, 1H), 8.9-9.0 (m, 1H), 8.5-8.5 (m, 1H), 8.44 (s, 1H), 6.5-6.6 (m, 1H), 5.5-5.7 (m, 1H), 2.84 (s, 3H), 2.6-2.7 (m, 1H), 2.61 (s, 3H), 0.8-0.9 (m, 2H), 0.15 (br d, 2H, J=5.6 Hz). MS: 476.2 ([M+H]⁺).

### Biological Examples

### Example 4

### 50% Growth Inhibitory Concentration (IC₅₀) Determination Assay:

The in vitro antimicrobial activity of the compounds against E. coli (BW25113, obtained from Coli Genetic Stock Center, #7636) and K. pneumoniae (ATCC10031) was determined according to the following procedure:

The assay used a 10-points Iso-Sensitest broth medium to measure quantitatively the in vitro activity of the compounds against E. coli BW25113 and K. pneumoniae ATCC 10031.

Stock compounds in DMSO were serially two-fold diluted (range from 50 to 0.097 μM final concentration) in 384 wells microtiter plates and inoculated with 49 μL the bacterial suspension in Iso-Sensitest broth medium to have a final cell concentration of ˜5×10⁵ CFU/mL in a final volume/well of 50 μL/well. Microtiter plates were incubated at 35±2° C.

Bacterial cell growth was determined with the measurement of optical density at k=600 nm each 20 minutes over a time course of 16 h.

Growth inhibition was calculated during the logarithmic growth of the bacterial cells with determination of the concentration inhibiting 50% (IC₅₀) of the growth.

GP-6 (Example 4.131 disclosed in WO 2012/125746 A1) was used as the reference compound in Table 5-10.

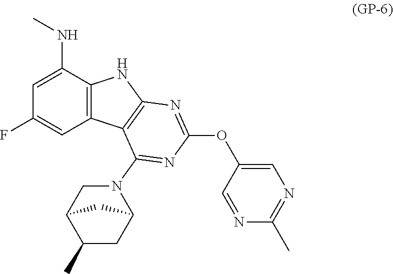 [SMILES: CNc1cc(F)cc2c1[nH]c1nc(Oc3cnc(C)nc3)nc(N3C[C@H]4C[C@@H]3C[C@H]4C)c12]

Compounds of the present invention were tested for their concentration inhibiting 50% (IC₅₀). The data of IC₅₀ over E. coli (BW25113) and K. pneumoniae (ATCC10031) are illustrated in Table 3. Particular compounds of the present invention were found to have IC₅₀≤1 μM.

TABLE 3
IC₅₀ values of the compounds of this invention
against E. coli and K. pneumoniae

| | IC50 (μM) | |
|---|---|---|
| | E. coli K12 | K. pneumoniae |
| Example No. | BW25113 | ATCC10031 |
| GP-6 | 0.170 | 0.098* |
| 1.01 | 0.906 | / |
| 1.02 | 0.262 | 0.098* |
| 1.03 | 2.711 | 0.114 |
| 1.05 | 2.064 | 0.221 |
| 1.06 | 0.828 | 0.187 |
| 1.07 | 0.885 | 0.319 |
| 1.09 | 2.568 | 0.284 |
| 1.10 | 3.577 | 0.098* |
| 1.11 | 3.124 | 0.098* |
| 1.12 | 0.442 | 0.098* |
| 1.13 | 2.857 | 0.098* |
| 1.14 | 8.578 | 0.197 |
| 1.15 | 0.663 | 0.098* |
| 1.16 | 1.970 | 0.183 |
| 1.17 | 0.497 | 0.098* |
| 1.21 | 4.780 | 0.155 |
| 1.23 | 0.458 | 0.098* |
| 1.24 | 2.230 | 0.105 |
| 1.32 | 0.828 | 0.187 |
| 2.01 | 0.523 | 0.098* |
| 2.02 | 0.738 | 0.098* |
| 2.03 | 0.748 | 0.098* |
| 2.04 | 1.214 | 0.220 |
| 3.01 | 0.808 | 0.110 |

*Detection limit

### Example 5

### Minimal Inhibitory Concentration Protocol (MIC) Assay:

The in vitro potency of compounds to inhibit E. coli (ATCC 25922) growth was assessed by MIC (Minimal Inhibitory Concentration) assay. Samples were prepared from 10 mM DMSO stock solutions. After serial 2-fold dilution in DMSO in a master plate (Greiner, Cat No: 651201), 180 μL sterile distilled water was added to 20 μL each aliquot of the samples. Then 104, diluted compounds were transferred into a new assay plate (Costar, 3599).

The growth medium Caution-Adjusted Mueller Hinton Broth (CAMHB) was prepared by adding the 20 mg per liter CaCl₂) and 20 mg per liter MgCl₂ into the MHB medium (Jianglai Company, sterilized).

Vials of each of the test microorganisms were maintained frozen in the vapor phase of a liquid nitrogen freezer. Took out the bacteria E. coli ATCC 25922 (KWIKSTIK, 0335K) from liquid nitrogen freezer, thawed it in room temperature, and diluted the bacterial in the CAMHB medium to achieve a final inoculum of 5×105 CFU/mL. 90 μL CAMHB with bacteria was dispensed to the assay plate and pipetted 5 times.

Then the assay plates were incubated for 20 hours at 35° C. in ambient air. Following incubation, the MIC (μg/mL), the lowest concentration of drug that inhibits visible growth of the microorganism was read and recorded throw the microscope.

TABLE 4
MIC values of the compounds
of this invention against E. coli

| | MIC (μg/mL) |
|---|---|
| Example | E. coli |
| No. | ATCC 25922 |
| GP-6 | 0.127 |
| 1.01 | 0.0183 |
| 1.02 | 0.598 |
| 1.03 | 0.682 |
| 1.05 | 0.576 |
| 1.06 | 0.122 |
| 1.07 | 0.0911 |
| 1.09 | 0.808 |
| 1.10 | 0.017 |
| 1.11 | 0.0716 |
| 1.12 | 0.0212 |
| 1.13 | 0.0909 |
| 1.15 | 0.152 |
| 1.16 | 0.169 |
| 1.17 | 0.0847 |
| 1.18 | 0.398 |
| 1.20 | 0.0707 |
| 1.21 | 0.392 |
| 1.23 | 0.0871 |
| 1.25 | 0.697 |
| 1.27 | 0.759 |
| 1.28 | 0.356 |
| 1.32 | 0.122 |
| 1.33 | 0.164 |
| 1.34 | 0.364 |
| 2.01 | 0.0223 |
| 2.02 | 0.0996 |
| 2.03 | 0.194 |
| 2.04 | 0.0432 |
| 2.06 | 0.84 |
| 3.01 | 0.594 |

### Example 6

### Lyophilisation Solubility Assay (LYSA):

The solubility of compounds of present invention was assessed by LYSA assay. Samples were prepared in duplicate from 10 mM DMSO stock solutions (20 μL) diluted in 30 μL pure DMSO. After evaporation of DMSO with a centrifugal vacuum evaporator, the residue was dissolved in 0.05 M phosphate buffer (150 μL, pH 6.5), stirred for one hour and shook for two hours. After one night, the solution was filtered using a microtiter filter plate. Then the filtrate and its 1/10 dilution were analyzed by HPLC-UV. In addition, a four-point calibration curve was prepared from the 10 mM stock solutions and used for the solubility determination of the compounds. The results were in μg/mL. and summarized in Table 5. Particular compounds of the present invention were found to have LYSA>10 μg/mL with much improved solubility compared to GP-6.

TABLE 5
Solubility data of Examples

| Example No. | Lysa (μg/mL) |
|---|---|
| GP-6 | <1.0* |
| 1.01 | 30 |
| 1.03 | 54 |
| 1.06 | 43 |
| 1.08 | 60 |
| 1.10 | 63 |
| 1.11 | 167 |
| 1.13 | 162 |
| 1.14 | 315 |
| 1.15 | 19 |
| 1.17 | 60 |
| 1.18 | 200 |
| 1.20 | 165 |
| 1.21 | 230 |
| 1.22 | 65 |
| 1.24 | 22 |
| 1.26 | >770 |
| 1.27 | 31 |
| 1.32 | 43 |
| 1.33 | 275 |
| 2.01 | 36 |
| 2.02 | 255 |
| 2.03 | 51 |
| 2.04 | 91 |
| 2.06 | >840 |

*Detection limit

### Example 7

### Cytotoxicity Assay:

The cytotoxicity of compounds of present invention was assessed by HepG2 assay. HepG2 cells (ATCC HB-8065™) were seeded into 96-well plates (1.2×104 cells per well) and treated with compounds for 3 days, cell viability was measured at day 3 post compound treatment using the CellTiter-Blue® Cell Viability Assay (Promega, Cat. No. G8082). 20 μL of Cell titer blue buffer were added to each well of the cells, incubated for 3 hours at 37° C., and the fluorescence value was measured by a plate reader (Molecular Device SpectraMax M2). The CC₅₀ value of each compound is plotted with GraphPad Prism using four parameter logistic equation. Results of cytotoxicity are given in Table 6.

TABLE 6
CC₅₀ of Examples

| Example No. | CC₅₀ (μM) |
|---|---|
| GP-6 | 5.6 |
| 1.01 | 45.89 |
| 1.03 | >100.00 |
| 1.05 | 33.97 |
| 1.09 | 33.45 |
| 1.10 | >100.00 |
| 1.11 | >100.00 |
| 1.12 | 29.88 |
| 1.13 | 80.08 |
| 1.14 | >100.00 |
| 1.18 | >100.00 |
| 1.24 | 71.00 |
| 1.26 | >100.00 |
| 1.31 | 30.81 |
| 1.34 | >100.00 |
| 2.05 | 31.76 |

*Detection limit

1. A compound of formula (I), 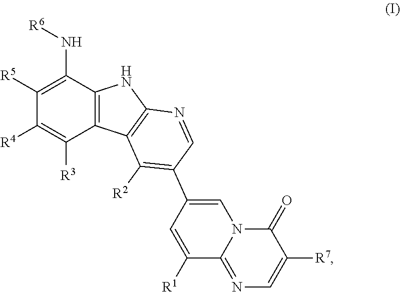
wherein
R¹ is H; (C₁₋₆alkyl)₂aminoC₁₋₆alkyl; C₁₋₆alkoxy; C₁₋₆alkoxyC₁₋₆alkyl(C₁₋₆alkyl)aminoC₁₋₆alkyl; C₁₋₆alkyl; C₁₋₆alkyl-2,3,3a,4,6,6a-hexahydropyrrolo[2,3-c]pyrrolylC₁₋₆alkyl; morpholinyl; morpholinylC₁₋₆alkyl or pyrrolidinylC₁₋₆alkyl;
R² is ((C₁₋₆alkyl)₂amino)C₁₋₆alkylhalopyrrolidinyl; ((C₁₋₆alkyl)₂amino)C₁₋₆alkylpyrrolidinyl; (C₁₋₆alkyl)₂amino; (C₁₋₆alkyl)₂aminopyrrolidinyl; C₁₋₆alkoxyC₁₋₆alkyl(C₁₋₆alkyl)amino; C₁₋₆ alkoxyC₁₋₆alkyl(C₁₋₆alkyl)aminoC₁₋₆alkylpyrrolidinyl; C₁₋₆alkyl-2,3,3a,4,6,6a-hexahydropyrrolo[2,3-c]pyrrolyl; C₃₋₇ cycloalkyl; C₃₋₇cycloalkyl(C₁₋₆alkyl)aminoC₁₋₆alkylpyrrolidinyl; cyano; haloC₁₋₆alkyl(C₁₋₆alkyl)amino; haloC₁₋₆alkyl(C₁₋₆alkyl)aminoC₁₋₆alkylpyrrolidinyl; haloC₁₋₆alkylpyrazolyl; halopyrrolidinyl; hexahydropyrazino[2,1-c][1,4]oxazin-8(1H)-yl; morpholinyl; phenylpyrrolidinyl or pyrrolidinyl;
R³ is H or halogen;
R⁴ is H or halogen;
R⁵ is H or halogen;
R⁶ is C₁₋₆alkyl;
R⁷ is carboxy;
or pharmaceutically acceptable salt thereof.

2. A compound according to claim 1, wherein
R¹ is H; dimethylaminomethyl; methoxy; methoxyethyl(methyl)aminomethyl; methyl; methyl-2,3,3a,4,6,6a-hexahydropyrrolo[2,3-c]pyrrolylmethyl; morpholinyl; morpholinylmethyl or pyrrolidinylmethyl;
R² is (dimethylamino)methyl(difluoro)pyrrolidinyl; (dimethylamino)methyl(fluoro)pyrrolidinyl; dimethylamino)methylpyrrolidinyl; cyano; cyclopropyl; cyclopropyl(methyl)aminomethylpyrrolidinyl; difluoroethyl(methyl)aminomethylpyrrolidinyl; difluoropyrrolidinyl; dimethylamino; dimethylaminopyrrolidinyl; hexahydropyrazino[2,1-c][1,4]oxazin-8(1H)-yl; methoxyethyl(methyl)amino; methoxyethyl(methyl)aminomethylpyrrolidinyl; methyl-2,3,3a,4,6,6a-hexahydropyrrolo[2,3-c]pyrrolyl; morpholinyl; phenylpyrrolidinyl; pyrrolidinyl; trifluoroethyl(methyl)amino or trifluoromethylpyrazolyl;
R³ is H, chloro or fluoro;
R⁴ is H, chloro or fluoro;
R⁵ is H or fluoro;
R⁶ is methyl or ethyl;
R⁷ is carboxy;
or pharmaceutically acceptable salt thereof.

3. A compound according to claim 1, or pharmaceutically acceptable salt thereof, wherein R¹ is H.

4. A compound according to claim 1, or pharmaceutically acceptable salt thereof, wherein R² is C₁₋₆alkyl-2,3,3a,4,6,6a-hexahydropyrrolo[2,3-c]pyrrolyl or (C₁₋₆alkyl)₂amino.

5. A compound according to claim 4, or pharmaceutically acceptable salt thereof, wherein R² is methyl-2,3,3a,4,6,6a-hexahydropyrrolo[2,3-c]pyrrolyl or dimethylamino.

6. A compound according to claim 1, wherein
R¹ is H;
R² is C₁₋₆alkyl-2,3,3a,4,6,6a-hexahydropyrrolo[2,3-c]pyrrolyl or (C₁₋₆alkyl)₂amino;
R³ is H or halogen;
R⁴ is H or halogen;
R⁵ is H or halogen;
R⁶ is C₁₋₆alkyl;
R⁷ is carboxy;
or pharmaceutically acceptable salt thereof.

7. A compound according to claim 6, wherein
R¹ is H;
R² is methyl-2,3,3a,4,6,6a-hexahydropyrrolo[2,3-c]pyrrolyl or dimethylamino;
R³ is H, chloro or fluoro;
R⁴ is H, chloro or fluoro;
R⁵ is H or fluoro;
R⁶ is methyl;
R⁷ is carboxy;
or pharmaceutically acceptable salt thereof.

8. A compound selected from:
7-[4-(dimethylamino)-5,6-difluoro-8-(methylamino)-9H-pyrido[2,3-b]indol-3-yl]-4-oxo-pyrido[1,2-a]pyrimidine-3-carboxylic acid;
7-[4-(dimethylamino)-5,6-difluoro-8-(methylamino)-9H-pyrido[2,3-b]indol-3-yl]-9-methyl-4-oxo-pyrido[1,2-a]pyrimidine-3-carboxylic acid;
7-[4-(dimethylamino)-5,6-difluoro-8-(methylamino)-9H-pyrido[2,3-b]indol-3-yl]-9-methoxy-4-oxo-pyrido[1,2-a]pyrimidine-3-carboxylic acid;
7-[4-(dimethylamino)-6-fluoro-8-(methylamino)-9H-pyrido[2,3-b]indol-3-yl]-4-oxo-pyrido[1,2-a]pyrimidine-3-carboxylic acid;
7-[4-(dimethylamino)-6-fluoro-8-(methylamino)-9H-pyrido[2,3-b]indol-3-yl]-9-methyl-4-oxo-pyrido[1,2-a]pyrimidine-3-carboxylic acid;
7-[4-(dimethylamino)-5,6-difluoro-8-(methylamino)-9H-pyrido[2,3-b]indol-3-yl]-9-morpholino-4-oxo-pyrido[1,2-a]pyrimidine-3-carboxylic acid;
7-[5,6-difluoro-8-(methylamino)-4-pyrrolidin-1-yl-9H-pyrido[2,3-b]indol-3-yl]-9-morpholino-4-oxo-pyrido[1,2-a]pyrimidine-3-carboxylic acid;
7-[5,6-difluoro-4-(5-methyl-2,3,3a,4,6,6a-hexahydropyrrolo[2,3-c]pyrrol-1-yl)-8-(methylamino)-9H-pyrido[2,3-b]indol-3-yl]-4-oxo-pyrido[1,2-a]pyrimidine-3-carboxylic acid;
7-(5,6-difluoro-4-(methyl(2,2,2-trifluoroethyl)amino)-8-(methylamino)-9H-pyrido[2,3-b]indol-3-yl)-4-oxo-4H-pyrido[1,2-a]pyrimidine-3-carboxylic acid;
7-(5,6-difluoro-8-(methylamino)-4-morpholino-9H-pyrido[2,3-b]indol-3-yl)-4-oxo-4H-pyrido[1,2-a]pyrimidine-3-carboxylic acid;
7-(6-fluoro-8-(methylamino)-4-morpholino-9H-pyrido[2,3-b]indol-3-yl)-4-oxo-4H-pyrido[1,2-a]pyrimidine-3-carboxylic acid;
7-(4-(dimethylamino)-6,7-difluoro-8-(methylamino)-9H-pyrido[2,3-b]indol-3-yl)-4-oxo-4H-pyrido[1,2-a]pyrimidine-3-carboxylic acid;
7-(6,7-difluoro-8-(methylamino)-4-morpholino-9H-pyrido[2,3-b]indol-3-yl)-4-oxo-4H-pyrido[1,2-a]pyrimidine-3-carboxylic acid;
7-(5,6-difluoro-8-(methylamino)-4-morpholino-9H-pyrido[2,3-b]indol-3-yl)-9-((dimethylamino)methyl)-4-oxo-4H-pyrido[1,2-a]pyrimidine-3-carboxylic acid;
7-(5,6-difluoro-8-(methylamino)-4-(pyrrolidin-1-yl)-9H-pyrido[2,3-b]indol-3-yl)-9-((dimethylamino)methyl)-4-oxo-4H-pyrido[1,2-a]pyrimidine-3-carboxylic acid;
7-(6-chloro-4-(dimethylamino)-8-(methylamino)-9H-pyrido[2,3-b]indol-3-yl)-4-oxo-4H-pyrido[1,2-a]pyrimidine-3-carboxylic acid;
7-(4-(dimethylamino)-5,7-difluoro-8-(methylamino)-9H-pyrido[2,3-b]indol-3-yl)-4-oxo-4H-pyrido[1,2-a]pyrimidine-3-carboxylic acid;
7-(4-(3-((dimethylamino)methyl)-3-fluoropyrrolidin-1-yl)-5,6-difluoro-8-(methylamino)-9H-pyrido[2,3-b]indol-3-yl)-4-oxo-4H-pyrido[1,2-a]pyrimidine-3-carboxylic acid;
7-(5,6-difluoro-4-(3-(((2-methoxyethyl)(methyl)amino)methyl)pyrrolidin-1-yl)-8-(methylamino)-9H-pyrido[2,3-b]indol-3-yl)-4-oxo-4H-pyrido[1,2-a]pyrimidine-3-carboxylic acid;
7-(4-(3-(((2,2-difluoroethyl)(methyl)amino)methyl)pyrrolidin-1-yl)-5,6-difluoro-8-(methylamino)-9H-pyrido[2,3-b]indol-3-yl)-4-oxo-4H-pyrido[1,2-a]pyrimidine-3-carboxylic acid;
7-(4-(3-((cyclopropyl(methyl)amino)methyl)pyrrolidin-1-yl)-6-fluoro-8-(methylamino)-9H-pyrido[2,3-b]indol-3-yl)-4-oxo-4H-pyrido[1,2-a]pyrimidine-3-carboxylic acid;
7-[4-[4-[(dimethylamino)methyl]-3,3-difluoro-pyrrolidin-1-yl]-5,6-difluoro-8-(methylamino)-9H-pyrido[2,3-b]indol-3-yl]-4-oxo-pyrido[1,2-a]pyrimidine-3-carboxylic acid;
7-(5-chloro-4-(dimethylamino)-6-fluoro-8-(methylamino)-9H-pyrido[2,3-b]indol-3-yl)-4-oxo-4H-pyrido[1,2-a]pyrimidine-3-carboxylic acid;
7-(5,6-difluoro-4-((2-methoxyethyl)(methyl)amino)-8-(methylamino)-9H-pyrido[2,3-b]indol-3-yl)-4-oxo-4H-pyrido[1,2-a]pyrimidine-3-carboxylic acid;
7-(6-fluoro-8-(methylamino)-4-(3-phenylpyrrolidin-1-yl)-9H-pyrido[2,3-b]indol-3-yl)-4-oxo-4H-pyrido[1,2-a]pyrimidine-3-carboxylic acid;
(S)-7-(4-(2-((dimethylamino)methyl)pyrrolidin-1-yl)-6,7-difluoro-8-(methylamino)-9H-pyrido[2,3-b]indol-3-yl)-4-oxo-4H-pyrido[1,2-a]pyrimidine-3-carboxylic acid;
(R)-7-(4-(3-(dimethylamino)pyrrolidin-1-yl)-6,7-difluoro-8-(methylamino)-9H-pyrido[2,3-b]indol-3-yl)-4-oxo-4H-pyrido[1,2-a]pyrimidine-3-carboxylic acid;
7-(5,6-difluoro-4-(hexahydropyrazino[2,1-c][1,4]oxazin-8(1H)-yl)-8-(methylamino)-9H-pyrido[2,3-b]indol-3-yl)-4-oxo-4H-pyrido[1,2-a]pyrimidine-3-carboxylic acid compound with formic acid;
7-[8-(ethylamino)-6-fluoro-4-[3-(trifluoromethyl)pyrazol-1-yl]-9H-pyrido[2,3-b]indol-3-yl]-9-methyl-4-oxo-pyrido[1,2-a]pyrimidine-3-carboxylic acid;
7-[8-(ethylamino)-6-fluoro-4-[3-(trifluoromethyl)pyrazol-1-yl]-9H-pyrido[2,3-b]indol-3-yl]-4-oxo-pyrido[1,2-a]pyrimidine-3-carboxylic acid;
8-[8-(ethylamino)-6-fluoro-4-[3-(trifluoromethyl)pyrazol-1-yl]-9H-pyrido[2,3-b]indol-3-yl]-4-oxo-pyrido[1,2-a]pyrimidine-3-carboxylic acid;
7-[4-(dimethylamino)-5,6-difluoro-8-(methylamino)-9H-pyrido[2,3-b]indol-3-yl]-9-morpholino-4-oxo-pyrido[1,2-a]pyrimidine-3-carboxylic acid;
7-[5,6-difluoro-4-(5-methyl-2,3,3a,4,6,6a-hexahydropyrrolo[2,3-c]pyrrol-1-yl)-8-(methylamino)-9H-pyrido[2,3-b]indol-3-yl]-9-methyl-4-oxo-pyrido[1,2-a]pyrimidine-3-carboxylic acid;
7-[4-cyano-6-fluoro-8-(methylamino)-9H-pyrido[2,3-b]indol-3-yl]-4-oxo-pyrido[1,2-a]pyrimidine-3-carboxylic acid;
7-[4-(dimethylamino)-5,6-difluoro-8-(methylamino)-9H-pyrido[2,3-b]indol-3-yl]-9-(morpholinomethyl)-4-oxo-pyrido[1,2-a]pyrimidine-3-carboxylic acid;
7-[4-(dimethylamino)-5,6-difluoro-8-(methylamino)-9H-pyrido[2,3-b]indol-3-yl]-9-[[2-methoxyethyl(methyl)amino]methyl]-4-oxo-pyrido[1,2-a]pyrimidine-3-carboxylic acid;
7-[4-(dimethylamino)-5,6-difluoro-8-(methylamino)-9H-pyrido[2,3-b]indol-3-yl]-4-oxo-9-(pyrrolidin-1-ylmethyl)pyrido[1,2-a]pyrimidine-3-carboxylic acid;
7-[5,6-difluoro-8-(methylamino)-4-pyrrolidin-1-yl-9H-pyrido[2,3-b]indol-3-yl]-9-(morpholinomethyl)-4-oxo-pyrido[1,2-a]pyrimidine-3-carboxylic acid;
7-(4-(3,3-difluoropyrrolidin-1-yl)-6-fluoro-8-(methylamino)-9H-pyrido[2,3-b]indol-3-yl)-4-oxo-4H-pyrido[1,2-a]pyrimidine-3-carboxylic acid;
7-[4-(dimethylamino)-5,6-difluoro-8-(methylamino)-9H-pyrido[2,3-b]indol-3-yl]-9-[(5-methyl-2,3,3a,4,6,6a-hexahydropyrrolo[2,3-c]pyrrol-1-yl)methyl]-4-oxo-pyrido[1,2-a]pyrimidine-3-carboxylic acid; and
7-[4-cyclopropyl-5,6-difluoro-8-(methylamino)-9H-pyrido[2,3-b]indol-3-yl]-9-methyl-4-oxo-pyrido[1,2-a]pyrimidine-3-carboxylic acid;
or pharmaceutically acceptable salt thereof.

9. A process for the preparation of a compound according to claim 1 comprising the reaction of compound of formula (Ii), 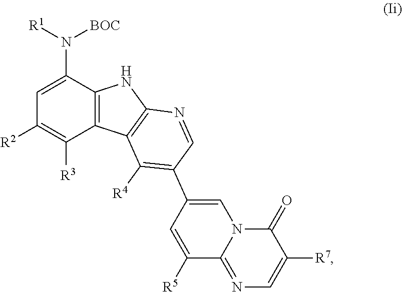
with an acid.

10. (canceled)

11. A pharmaceutical composition comprising a compound of claim 1 and a therapeutically inert carrier.

12-16. (canceled)

17. A compound according to claim 1 when manufactured according to a process of claim 9.

18. A method for the treatment of bacterial infection, which method comprises administering an effective amount of a compound of claim 1.

19. A compound of claim 1, wherein at least one of R³ or R⁵ is H.

20. A compound of claim 1, wherein R⁴ is fluoro;

21. A compound of claim 1, wherein R⁶ is methyl.

22. A compound according to claim 1, wherein
R² is ((C₁₋₆alkyl)₂amino)C₁₋₆alkylhalopyrrolidinyl; ((C₁₋₆alkyl)₂amino)C₁₋₆alkylpyrrolidinyl; (C₁₋₆alkyl)₂aminopyrrolidinyl; C₁₋₆alkoxyC₁₋₆ alkyl(C₁₋₆alkyl)aminoC₁₋₆alkylpyrrolidinyl; C₃₋₇cycloalkyl(C₁₋₆alkyl)aminoC₁₋₆alkylpyrrolidinyl; haloC₁₋₆alkyl(C₁₋₆alkyl)aminoC₁₋₆alkylpyrrolidinyl; halopyrrolidinyl; phenylpyrrolidinyl or pyrrolidinyl.

23. A compound according to claim 1, wherein
R² is (C₁₋₆alkyl)₂amino; C₁₋₆alkoxyC₁₋₆alkyl(C₁₋₆alkyl)amino; or haloC₁₋₆alkyl(C₁₋₆alkyl)amino.

24. A pharmaceutical composition comprising a compound of claim 8 and a therapeutically inert carrier.

25. A method for the treatment of bacterial infection, which method comprises administering an effective amount of a compound of claim 8.

26. The method of claim 9 wherein the acid is trifluoroacetic acid.
